(19) United States
(12) Patent Application Publication
(10) Pub. No.: US 2005/0164234 A1
(43) Pub. Date: Jul. 28, 2005

(54) Methods of using mammalian RNase H and compositions thereof

(76) Inventors: Stanley T. Crooke, Carlsbad, CA (US); Walter F. Lima, San Diego, CA (US); Hongjiang Wu, Carlsbad, CA (US)

Correspondence Address: LICATA & TYRRELL P.C., 66 E. MAIN STREET, MARLTON, NJ, 08053, US

(21) Appl. No.: 10/943,194

(22) Filed: Sep. 16, 2004

Related U.S. Application Data
(63) Continuation of application No. PCT/US04/27348, filed on Aug. 20, 2004.
Application No. PCT/US04/27348 is a continuation-in-part of application No. 10/679,791, filed on Oct. 6, 2003.
Application No. 10/679,791 is a continuation-in-part of application No. 10/358,439, filed on Feb. 3, 2003.
Application No. 10/358,439 is a continuation-in-part of application No. 09/861,205, filed on May 18, 2001, now abandoned.
Application No. 09/861,205 is a continuation of application No. 09/684,254, filed on Oct. 6, 2000, now Pat. No. 6,376,661.
Application No. 09/684,254 is a continuation of application No. 09/343,809, filed on Jun. 30, 1999, now abandoned.
Application No. 09/343,809 is a continuation of application No. 09/203,716, filed on Dec. 2, 1998, now Pat. No. 6,001,653.
(60) Provisional application No. 60/067,458, filed on Dec. 4, 1997.
(60) Provisional application No. 60/527,413, filed on Dec. 4, 2003.

(51) Int. Cl. C12Q 1/68; C12P 19/34; C12N 9/22

(52) U.S. Cl. 435/6; 435/91.2; 435/199

(57) ABSTRACT

The present invention relates to methods for using mammalian RNase H, including human RNase H, and compositions thereof, particularly for reduction of a selected cellular RNA target via antisense technology. Methods and uses for increasing or decreasing RNase H levels and activity in cells and animals are disclosed.

This application is a continuation of U.S. Ser. No. PCT/US2004/027348 filed Aug. 20, 2004, which is a continuation-in-part of U.S. Ser. No. 10/679,761 filed Aug. 20, 2004, which is a continuation-in-part of U.S. Ser. No. 10/358,439 filed Feb. 3, 2003, which is a continuation-in-part of U.S. Ser. No. 09/861,205 filed May 18, 2001, now abandoned, continuation of U.S. Ser. No. 09/684,254 filed Oct. 6, 2000, now issued as U.S. Pat. No. 6,376,661, which is a continuation of U.S. Ser. No. 09/343,809 filed Jun. 30, 1999, now abandoned, which is a continuation of U.S. Ser. No. 09/203,716 filed Dec. 2, 1998, now issued as U.S. Pat. No. 6,001,653, which claims the benefit of priority of U.S. Ser. No. 60/067,458 filed Dec. 4, 1997.

This application also claims the benefit of U.S. Ser. No. 60/527,413 filed Dec. 4, 2003 and U.S. Ser. No. 60/497,412 filed Aug. 21, 2003.

### FIELD OF THE INVENTION

The present invention relates to methods for using mammalian RNase H and compositions thereof, particularly for reduction of selected cellular RNA via antisense technology. Modulation of RNase H levels and/or activity is also provided, as are novel assays and methods for detection of RNase H.

### BACKGROUND OF THE INVENTION

RNase H hydrolyzes RNA in RNA-DNA hybrids. This enzymatic activity was first identified in calf thymus but has subsequently been described in a variety of organisms (Stein, H. and Hausen, P., Science, 1969, 166, 393-395; Hausen, P. and Stein, H., Eur. J. Biochem., 1970, 14, 278-283). RNase H activity appears to be ubiquitous in eukaryotes and bacteria (Itaya, M. and Kondo K. Nucleic Acids Res., 1991, 19, 4443-4449; Itaya et al., Mol. Gen. Genet., 1991 227, 438-445; Kanaya, S., and Itaya, M., J. Biol. Chem., 1992, 267, 10184-10192; Busen, W., J. Biol. Chem., 1980, 255, 9434-9443; Rong, Y. W. and Carl, P. L., 1990, Biochemistry 29, 383-389; Eder et al., Biochimie, 1993 75, 123-126). Although RNases H constitute a family of proteins of varying molecular weight, nucleolytic activity and substrate requirements appear to be similar for the various isotypes. For example, all RNases H studied to date function as endonucleases, exhibiting limited sequence specificity and requiring divalent cations (e.g., Mg²⁺, Mn²⁺) to produce cleavage products with 5′ phosphate and 3′ hydroxyl termini (Crouch, R. J., and Dirksen, M. L., Nuclease, Linn, S, M., & Roberts, R. J., Eds., Cold Spring Harbor Laboratory Press, Plainview, N.Y. 1982, 211-241).

In addition to playing a natural role in DNA replication, RNase H has also been shown to be capable of cleaving the RNA component of certain oligonucleotide-RNA duplexes. While many mechanisms have been proposed for oligonucleotide mediated destabilization of target RNAs, the primary mechanism by which antisense oligonucleotides are believed to cause a reduction in target RNA levels is through this RNase H action. Monia et al., J. Biol. Chem., 1993, 266:13, 14514-14522. In vitro assays have demonstrated that oligonucleotides that are not substrates for RNase H can inhibit protein translation (Blake et al., Biochemistry, 1985, 24, 6139-4145) and that oligonucleotides inhibit protein translation in rabbit reticulocyte extracts that exhibit low RNase H activity. However, more efficient inhibition was found in systems that supported RNase H activity (Walder, R. Y. and Walder, J. A., Proc. Nat'l Acad. Sci. USA, 1988, 85, 5011-5015; Gagnor et al., Nucleic Acid Res., 1987, 15, 10419-10436; Cazenave et al., Nucleic Acid Res., 1989, 17, 4255-4273; and Dash et al., Proc. Nat'l Acad. Sci. USA, 1987, 84, 7896-7900.

RNase HI from E. coli is the best-characterized member of the RNase H family. The 3-dimensional structure of E. coli RNase HI has been determined by x-ray crystallography, and the key amino acids involved in binding and catalysis have been identified by site-directed mutagenesis (Nakamura et al., Proc. Natl. Acad. Sci. USA, 1991, 88, 11535-11539; Katayanagi et al., Nature, 1990, 347, 306-309; Yang et al., Science, 1990, 249, 1398-1405; Kanaya et al., J. Biol. Chem., 1991, 266, 11621-11627). The enzyme has two distinct structural domains. The major domain consists of four α helices and one large β sheet composed of three antiparallel β strands. The Mg²⁺ binding site is located on the β sheet and consists of three amino acids, Asp-10, Glu-48, and Gly-11 (Katayanagi et al., Proteins: Struct., Funct., Genet., 1993, 17, 337-346). This structural motif of the Mg²⁺ binding site surrounded by β strands is similar to that in Dnase I (Suck, D., and Oefner, C., Nature, 1986, 321, 620-625). The minor domain is believed to constitute the predominant binding region of the enzyme and is composed of an α helix terminating with a loop. The loop region is composed of a cluster of positively charged amino acids that are believed to bind electrostatistically to the minor groove of the DNA/RNA heteroduplex substrate. Although the conformation of the RNA/DNA substrate can vary from A-form to B-form depending on the sequence composition, in general RNA/DNA heteroduplexes adopt an A-like geometry (Pardi et al., Biochemistry, 1981, 20, 3986-3996; Hall, K. B., and Mclaughlin, L. W., Biochemistry, 1991, 30, 10606-10613; Lane et al., Eur. J. Biochem., 1993, 215, 297-306). The entire binding interaction appears to comprise a single helical turn of the substrate duplex. Recently the binding characteristics, substrate requirements, cleavage products and effects of various chemical modifications of the substrates on the kinetic characteristics of E. coli RNase HI have been studied in more detail (Crooke, S. T. et al., Biochem. J., 1995, 312, 599-608; Lima, W. F. and Crooke, S. T., Biochemistry, 1997, 36, 390-398; Lima, W. F. et al., J. Biol. Chem., 1997, 272, 18191-18199; Tidd, D. M. and Worenius, H. M., Br. J. Cancer, 1989, 60, 343; Tidd, D. M. et al., Anti-Cancer Drug Des., 1988, 3, 117.

In addition to RNase HI, a second E. coli RNase H, RNase HII, has been cloned and characterized (Itaya, M., Proc. Natl. Acad. Sci. USA, 1990, 87, 8587-8591). It is comprised of 213 amino acids while RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449; Itaya et al., Mol. Gen. Genet., 1991, 227, 438-445). An enzyme cloned from S. cerevisae was 30% homologous to E. coli RNase HI (Itaya, M. and Kondo K., Nucleic Acids Res., 1991, 19, 4443-4449; Itaya et al., Mol. Gen. Genet., 1991, 227, 438-445).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have so far been defined based on differences in molecular weight, effects of divalent cations, sensitivity to sulfhydryl agents and immunological cross-reactivity (Busen et al., Eur. J. Biochem., 1977, 74, 203-208). RNase H2 enzymes (also called RNase HII, formerly called Type 1 RNase H) are reported to have molecular weights in the 68-90 kDa range, be activated by either Mn²⁺ or Mg²⁺ and be insensitive to sulfhydryl agents. In contrast, RNase H1 enzymes (also called RNase HI, formerly called Type 2 RNase H) have been reported to have molecular weights ranging from 31-45 kDa, to require Mg^{2+,} to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108.).

An enzyme with Type 2 RNase H (i.e., RNase H1) characteristics has been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

Multiple mammalian RNases H have recently been cloned, sequenced and expressed. These include human RNase H1 [Crooke et al., U.S. Pat. No. 6,001,653; Wu et al., Antisense Nucl. Acid Drug Des. 1998, 8: 53-61; Genbank accession no. AF039652; Cerritelli and Crouch, 1998, Genomics 53, 300-307; Frank et al., 1998, Biol. Chem. 379, 1407-1412], human RNase H2 [(Frank et al., 1998, Proc. Natl. Acad. Sci. USA 95, 12872-12877)] and other mammalian RNases H (Cerritelli and Crouch, ibid.). The availability of purified RNase H has facilitated efforts to understand the structure of the enzyme, its distribution and the function(s) it may serve.

Many of the properties observed for Human RNase H1 are consistent with the E. coli RNase H1 isotype, (e.g., the cofactor requirements, substrate specificity and binding specificity) H1. Wu et al., 1999, J. Biol. Chem. 274, 28270-28278; Lima, W. F. and Crooke, S. T., 1997, Biochemistry 36, 390-398. In fact, the carboxy-terminal portion of human RNase H1 is highly conserved with the amino acid sequence of the E. coli enzyme, (region III). The glutamic acid and two aspartic acid residues of the catalytic site, as well as the histidine and aspartic acid residues of the proposed second divalent cation binding site of the E. coli enzyme are conserved in human RNase H1. Kanaya et al., 1991, J. Biol. Chem., 266, 11621-11627; Nakamura et al., 1991, Proc. Natl. Acad. Sci. U.S.A., 88, 11535-11539; Katanagi et al., 1990, Nature, 347, 306-309; Yang et al., 1990, Science 249, 1398-1405. In addition, the lysine residues within the highly basic α-helical substrate-binding region of E. coli RNase H1 are also conserved in the human enzyme.

Despite these similarities, the structures of the two enzymes differ in several important ways. For example, the amino acid sequence of the human enzyme is approximately 2-fold larger than the E. coli enzyme. The additional amino acid sequence of the human enzyme extends from the amino-terminus of the conserved E. coli RNase H1 region and contains a 73 amino acid region homologous with a double-strand RNA (dsRNA) binding motif, (region I). The conserved E. coli RNase H1 region at the carboxy-terminus is separated from the dsRNA-binding domain of the human enzyme, by a 62 amino acid region, (region II). Thus the human RNase H1 protein can be divided into three regions. Region I, located at the amino-terminus of the enzyme, contains a structure consistent with a dsRNA-binding motif. Region II consists of a 62 amino acid region between the dsRNA-binding domain and the conserved E. coli RNase H1 region. Lastly, region III is situated at the carboxy-terminus of human RNase H1 and contains an amino acid sequence that is highly conserved with the amino acid sequence of E. coli RNase H1. Included within region III are the conserved amino acid residues that form the putative catalytic site, the second divalent cation binding site, and the basic substrate-binding domain of the E. coli enzyme.

The three amino acids (Asp-10, Glu-48 and Asp-70) that make up the catalytic site of E. coli RNase H1 were identified by site-directed mutagenesis (Katanagi et al., 1990, Nature 347, 306-309). These amino acid residues have also been shown to be involved with the coordination of the requisite divalent cation cofactor. Katayanagi et al., 1993, Proteins: Struct. Funct, Genet. 17, 337-346. Comparison of the amino acid sequence of E. coli RNase H1 with the amino acid sequences of the RNase H domain of retroviruses and RNase H1 from yeast, chicken, Human and mouse indicates that these three amino acid residues are conserved among all type 1 sequences. Wu et al., 1998, Antisense Nucl. Acid Drug Dev., 8, 53-61. Although the role of both regions I and II remain unclear, the dsRNA-binding domain of human RNase H1 may account for the observed positional preference for cleavage displayed by the enzyme as well as the enhanced binding affinity of the enzyme for various polynucleotides. Wu et al., 1999, J. Biol. Chem. 274, 28270-28278.

The present invention provides modulation of mammalian RNase H levels and/or activity via several approaches.

### SUMMARY OF THE INVENTION

The present invention generally provides compositions and methods for modulating the activity or expression of a mammalian RNase H in a cell or mammal. Methods of enhancing antisense effects by modulating RNase H levels or activity are also provided. For the foregoing methods the RNase H may be exogenously added or overexpressed by means of a vector; may be wild type or mutant forms of the enzyme, and may be RNase H1 or H2. The RNase H may be Fen1 which has been shown herein to have RNase H activity. The expression of the RNase H may be reduced by use of antisense compounds which specifically inhibit the expression of the RNase H. The antisense compounds may be oligonucleotides, including DNA or RNA oligonucleotides, both single- and double-stranded. Also provided are mammalian cells and non-human mammals comprising a nucleic acid encoding a human RNase H polypeptide. Methods of modulating the potency of one or more antisense compounds in a mammalian cell or a mammal comprising modulating the amount of RNase H1 in said cell or mammal. In some embodiments antisense potency is increased by increasing the amount of active RNase H1 in the cell or mammal. In other embodiments antisense potency is decreased by decreasing the amount of active mammalian RNase H1 in the cell or mammal. Also provided is a substantially isolated and purified large human RNase H and methods of cleaving the RNA strand of an RNA/DNA duplex with said enzyme. Fen1 is an example of such a large human RNase H which cleaves the RNA strand of an RNA/DNA duplex.

A substantially isolated and purified cloned and expressed mammalian RNase H2 which retains cleavage activity for a RNA/DNA duplex substrate is also provided.

### BRIEF DESCRIPTION OF THE DRAWINGS

FIG. 1. The human RNase H1 primary sequence (286 amino acids; SEQ ID NO: 1) and sequence comparisons with chicken (293 amino acids; SEQ ID NO: 2), yeast (348 amino acids; SEQ ID NO: 3) and E. coli RNase H1 (155 amino acids; SEQ ID NO: 4) as well as an EST deduced mouse RNase H homolog (GenBank accession no. AA389926 and AA518920; SEQ ID NO: 5). Boldface type indicates amino acid residues identical to human. “@” indicates the conserved amino acid residues implicated in E. coli RNase H1 Mg²⁺ binding site and catalytic center (Asp-10, Gly-11, Glu-48 and Asp-70). “*” indicates the conserved residues implicated in E. coli RNases H1 for substrate binding.

FIG. 2. A novel human RNase H2 primary sequence (299 amino acids; SEQ ID NO: 6) and sequence comparisons with mouse (SEQ ID NO: 7), C. elegans (SEQ ID NO: 8), yeast (300 amino acids; SEQ ID NO: 9) and E. coli RNase HII (298 amino acids; SEQ ID NO: 10). Boldface type indicates amino acid residues identical to human. Uppercase letters above alignment indicate amino acid residues identically conserved among species; lower case letters above alignment indicate residues similarly conserved.

FIG. 3. Schematic diagram showing the structure of human RNase H1 mutant proteins. FIG. 3A shows the position of amino acid substitution mutants. Mutants include: aspargine substitution of aspartic acid at position 145 [D145 N], glutamine substitution of glutamic acid at position 186 [E186Q], aspargine substitution of Aspartic acid at position 210 [D210 N], alanine substitution of lysine at positions 226 and 227 [K226,227A] and alanine substitution of lysine at positions 226, 227, 231 and 236 [K226,227,231,236A]. The amino acids of regions I, II and II are represented by, respectively, in bold, underlined and plain lettering. Designations within parentheses indicate amino acid positions of E. coli RNase H1. FIG. 3B is a schematic of deletion mutants of human RNase H1. RNase H1[ΔI] corresponds to the deletion of region I (amino acid positions 1-73), RNase H1[ΔII] corresponds to the deletion of region II (amino acid positions 74-135) and RNase H1[ΔI-II] corresponds to the deletion of regions I and II (amino acid positions 1-135).

FIG. 4. Development of adenoviruses over-expressing human RNase H's. FIG. 4A. Human RNase H constructs in adenovirus shuttle vectors. Full length and N-terminal 26 amino acid (suggested mitochondria localization signal, MLS) minus RNase H1, and full length RNase H2 cDNAs were amplified by PCR and cloned into EcoRI and XhoI sites in the multiple cloning site (MCS) downstream from the CMV promoter in the adenovirus shuttle vector, pACCCMVpLpA(−)Loxp-ssp (Core facility of University of Michigan). The RNase H1 virus may use the first or the second (amino acid 27) methionine (Met1 or Met27) to start protein translation. FIG. 4B. Western blot analysis on protein lysates from HeLa or A549 cells infected with full length H1 or H2 virus (200 pfu/cell). The cells were harvested at different time points (0, 6, 12, 24, 36, 48 and 72 hours) after virus infection. The protein concentrations of the cell lysates were measured. The lysates were subjected to 4-20% gradient SDS-PAGE (20 ug/lane) and western blot analysis with anti RNase H1 (against H1 C-terminal peptides, see method) and H2 antibodies. FIG. 4C. Immunoprecipitation (IP) with purified H1 Ab (against partial RNase H1 protein). (1) IP was performed using untreated HeLa cell lysate with purified H1 Ab which was covalently immobilized to agarose beads. The eluted samples were subjected to the western blot analysis with H1 Ab. (2) HeLa cells were infected with full length or N-terminal 26 amino acid minus RNase H1 virus or control virus (LoxP). Cell lysates were prepared after 24 hours of infection and subjected to IP with H1 Ab (10 ug Ab/mg protein lysate). The samples from IP were further analyzed by SDS-PAGE and western blot with H1 Ab.

FIG. 5. Gel renaturation assay on (1) uninfected HeLa cell lysate (5 ug); (2) samples from IP with H1 Ab from HeLa cell nuclear and cytosolic extracts (see methods); (3) samples from IP with H2 Ab from the lysates of HeLa cells infected without or with H2 or control virus; (4) samples from IP with H1 Ab from the lysates of HeLa cells infected without or with H1 or control virus.

FIG. 6. Human RNase H1 and H2 Cleavage. FIG. 6A: RNase H Ab immunoprecipitation (IP) coupled TCA assay. HeLa cells were infected with human RNase H1, H2 or control virus (200 pfu/cell) in 10 cm plate in quadruplicate for 24 hours before harvest. Cell lysates were prepared and protein concentrations measured. 0.7 mg protein lysate was used for H1 Ab IP (15 ug H1 Ab/mg protein lysate) or 0.35 mg per tube for H2 Ab IP (30 ug H2 Ab/mg protein). One set of the IP samples was eluted in 2×SDS loading buffer (Invitrogen Inc. San Diego) and subjected to SDS-PAGE and western blot with H1 or H2 Abs. The other three sets of IP samples were used in the enzyme activity assay (TCA precipitation) against a 50 nM of a 17 mer RNA/DNA duplex. The ASOs were hybridized with 5′-end-labeled sense oliogribonucleotides (17 mer, encoding a human RAS sequence, see examples), then digested with the IP samples for different lengths of time at 37° C. The digested duplexes were subjected to TCA precipitation (see methods) and the radioactivity in supernatants was determined for the digested RNA fragments by scintillation counting. The experiments were performed in triplicate and repeated three times. The bars show the standard error of the mean.

FIG. 7. Different digestion patterns of human RNase H1 and H2. Two different RNA/DNA substrates (17 mer RAS and 20 mer human Bclx sequences) were prepared and subjected to digestion by the H1 or H2 Ab IP samples from untreated HeLa cells for different time periods at 37° C. The digested duplexes were subjected to denaturing polyacrylamide gel analysis. Panel A. (Left) Cleavage of 17 mer RAS duplex. The asterisks indicate the major differences in cleavage sites between RNase H1 and H2. (right) The relative extents of digestions at each position of the substrate were calculated with the phosphor-Imager and the relative percentage of digestion are compared between RNase H1 and H2. Panel B. Cleavage of 20 mer Bclx duplex.

FIG. 8. Effects of RNase H1 and H2 over-expression on the potency of DNA-like ASOs. HeLa cells were split into 6000 cells/well in 96 well plates, then infected with H1, H2 or control (LoxP) viruses (200 pfu/cell). 12 hours later, the cells were transfected with the anti-cRaf (FIG. 8A) ASO (ISIS 13650) at different concentrations. The cells were harvested 24 hours later. cRaf mRNA level were measured with RT-PCR in which the reverse transcription and PCR amplification of cRaf mRNA were performed in 96 well format with the primer set described in methods. The IC50s were calculated and presented under the graphs. The bars represent standard error of the mean of 3-5 replicates of a representative experiment. FIG. 8B: A similar experiment with anti PTEN ASO (ISIS 116847). FIG. 8C: with anti-JNK2 ASO (ISIS 101759). FIG. 8D: A similar experiment in a A549 cells. FIG. 8E: Northern blot analyses of the effects of RNase H1 on the potency of the cRaf ASO in HeLa cells. The cells were split into 10e6 cells per 10 cm plate and incubated with control or H1 virus (200 pfu/cell) for 12 hours before the cells were transfected with anti-c-Raf ASO (ISIS 13650) of different concentration via lipofectin (see method). The cells were harvested 24 hours later and the total RNA was prepared with RNAeasy kit (Quiagen, Germany). 5 ug RNA/lane was subjected to 1.2% agarose/formaldehyde and further to Northern blot analysis with 32P labeled human cRaf cDNA probe and house keeping gene G3PDH (G3) probe (for normalization). The experiment was performed in triplicate and results were plotted with percentage normalized mRNA level versus ASO concentration. The bars represent standard error of the mean of the triplicates. The experiment was repeated several times.

FIG. 9. Overexpression of the dominant negative RNase H1 and H2. Overexpression of the dominant negative #48 E->Q RNase H1 mutant reduces antisense activity of antisense in human cell lines (FIG. 9A). Overexpression of a dominant negative mutant of RNase H2 had no effect on antisense activity in cells (FIG. 9B).

FIG. 10. Overexpression of human RNase H1 enhances ASO activity in mouse cell lines. FIG. 10A. Over-expression of human RNase H1 in mouse AML12 and Hepa cell lines. Adenoviral infection and western blot analyses were performed as described in examples. FIG. 10B. Over-expression of human RNase H1 increases anti-mouse JNK1 ASO potency.

FIG. 11. Effects of overexpression of human RNase H1 on Fas ASO potency in mouse liver. FIG. 11A. Analysis of expression of human RNase H1 in mouse liver. Mice were treated with different amounts of Fas ASO (ISIS 22023) and then the H1 or control viruses 4 hours later as indicated in the figure. After another 24 hours, the animals were sacrificed and the livers harvested. Liver tissue lysate was prepared with SDS RIPA lysis buffer (see method). 20 ug protein were used in the gel renaturation assay (GRN) in the presence of 10 mM Mg⁺⁺ (see C) and Western Blot (WB) with anti human RNase H1 Ab. Each lane represents a sample from an individual animal (n=4 for each group). FIG. 11B. RNA protection assay. Total RNA was extracted from the livers of the same mice as in FIG. 11A. The expression of Fas mRNA in liver was determined by an RNA protection assay (RPA). Each lane in the gel represents a sample from an individual animal. The figure shows only two lanes for each group (n=4). Fas and other RNAs are labeled to the left of the figure. FIG. 11C. Effects of different doses of Fas ASO on Fas mRNA levels were compared with saline control group after normalization to L32 (mRNA) mRNA expression, respectively. The bars represent the standard error of the mean of four animals in each group. This experiment was repeated three times with equivalent results.

FIG. 12. ASO or siRNA reduction of RNase H1 and H2 in HeLa and A549 cell lines.

FIGS. 12A & B. ASO ISIS 194178 or si-H1 reduces RNase H1 mRNA levels and enzyme activity. FIGS. 12C & D. ASO ISIS 194186 or si21956 reduces RNase H2 mRNA and protein levels. Cells were treated with different amounts of ASO or siRNA for 24 hours. Total RNA and cell lysates were prepared. As described earlier, the RNA was subjected to 1.2% agarase/formaldehyde gel (5 ug total RNA/lane) and Northern blot analysis with 32P-labeled human RNase H1 or H2 or a G3PDH cDNA probe. 20 ug proteins of cell lysate were used for gel renaturation assay to test RNase H1 activity or for Western blot with anti human RNase H2 Ab.

FIG. 13. Effects of siRNA to RNase H1 or RNase H2 on the potency of cRaf antisense oligonucleotide (ISIS 13650) in HeLa (and A549 Cells). FIG. 13A. Effects of RNase H1 siRNA on the potency of the antisense oligonucleotide in HeLa cells. Cells were first transfected with various concentrations of RNase H1 siRNA as indicated for 10 hours before the cells were split into 96 well format cell culture plates (6000 cells/well) and incubated for 10-14 hours. The cells were transfected with various concentrations of ISIS 13650 for 24 hours before harvest. Total RNAs were prepared and the cellular c-Raf and RNase H1 mRNA levels were determined with RT-PCR in which the reverse transcription and PCR amplification of c-Raf and RNase H1 mRNAs were performed in the 96 well format with the primer sets described in the examples. The vertical bars represent standard error of the mean of 3-6 replicates of a representative experiment. A1. Reduction of cellular RNase H1 by H1 siRNA. A2. Effects of RNase H1 siRNA treatment on the potency of c-Raf ASO (ISIS 13650). The IC50s were calculated and presented under the graph. A3. Correlation of cellular RNase H1 mRNA levels with the potency of ISIS 13650. Cellular RNase H mRNA levels were determined by RT-PCR as described. The RNase H1 mRNA levels in arbitrary results for untreated cells were divided by level of the RNase H1 mRNA from treated cells to obtain the relative level of RNase H1 RNA. Percent reduction of c-Raf RNA was calculated as previously described. FIG. 13B. Effects of reduction of cellular RNase H2 by siRNA on the potency of the antisense oligonucleotide in HeLa cells. Similar methods as described in FIG. 12A with the RNase H2 siRNA pretreatment. FIG. 13C. The effects of various ratios of siRNA to RNase H1 and RNase H2 on the potency of ISIS 13650. The experimental methods are as described except that the total concentration of siRNA was maintained at 25 nM and the ratio of H1 siRNA to H2 siRNA was varied from 0 to 1. The vertical bars represent the SEM of the mean of 3 replicates of a representative experiment.

FIG. 14. Effects of antisense oligonucleotide reduction of RNase H1 and H2 (ISIS 194178 and ISIS 194186) on c-Raf antisense oligonucleotide (ISIS 13650) potency in HeLa and A549 cells. A&B. Reduction of c-Raf mRNA in HeLa cells (FIG. 14A) and A549 cells (FIG. 14B) pre-treated with antisense to RNase H1 or H2. Methods as in FIG. 13 A&B and D. Each antisense oligonucleotide was transfected at 150 nM concentration. The vertical bars represent standard error of the mean of 6 replicates of a representative experiment.

FIG. 15. Several RNase Hs are present in human cells. Cell lysates were prepared in RIPA lysis buffer from human HeLa, A549, T24, MCF7 and HepG2 cells as described in methods. 20 ug protein from each lysate were used in gel renaturation assay (see methods). Lanes 1-2: HeLa cell lysates; Lanes 3-4: A549 lysates; Lane 5-6: T24; Lane 7: MCF7 and Lane 8: HepG2 lysate. The lysates from Lane 2, 4 and 6 were prepared with the lysis buffer without phosphatase inhibitors. FIG. 15A. Gel renaturation assay in the presence of Mg⁺². FIG. 15B. Gel renaturation in the presence of Mn⁺². This is a representative experiment that has been repeated more than 5 times.

FIG. 16. Several RNase Hs are present in human cells. FIG. 16A. Gel renaturation assay in the presence of Mg⁺⁺ of HeLa cell lysates prepared as described in methods. Prior to preparation of the lysates, the HeLa cells were treated with either a control oligonucleotide or the RNase H1 antisense oligonucleotide (ISIS 194178) at the concentrations indicated. This is a representative experiment repeated more than 3 times. FIG. 16B. Gel renaturation assay in the presence of Mn⁺⁺ of A549 cell lysates. Cells were treated with a control or the siRNA for RNase H1 as indicated. FIG. 16C. Western blot analysis of RNase H1 from HeLa cell lysates. Cell lystates were prepared as previously described. These were subjected to immunoprecipitation with the purified polyclonal antibodies to human RNase H1. The supernatant was separated from the protein A beads by centrifugation. All samples were then subjected to SDS-PAGE and probed with the purified human RNase H1 antibody. FIG. 16D. Gel renaturation assay of the HeLa cell lysates after immunoprecipitation.

### DETAILED DESCRIPTION OF THE INVENTION

The present invention relates to mammalian RNase H, particularly human RNase H1 and human RNase H2. Alteration of levels and/or activity of RNase H via a number of approaches are described, as are methods for use of RNase H. In addition to RNase H1 and H2, several higher and lower molecular weight protein bands from HeLa cell lysates were observed to have RNase H activity, particularly in the presence of Mg2+ or Mn2+. Purification and mass spec analysis of the larger (apparent molecular weight 50-70 kD on gel renaturation assay) RNase H band indicated that the band contained flap structure-specific endonuclease 1 (Fen1, NCBI gi number 4758356. This 380-amino acid (calculated molecular weight approximately 42 kDa) protein cleaves DNA flap strands that terminate with a 5′ single-stranded end and is known to remove 5′ overhanging flaps in DNA repair and process the 5′ ends of Okazaki fragments in lagging strand DNA synthesis. Rumbaugh et al., 1999, J. Biol. Chem., 274, 14602-14608.

To determine whether Fen1 was actually responsible for the RNase H activity seen on the gel renaturation assay, the Fen1 enzyme was immunoprecipitated from HeLa cells and was found to yield an RNase H activity band on the gel renaturation assay at the expected molecular mass position of approximately 50 kD.

Cloned human Fen1 was subsequently shown to have RNase activity at the appropriate size position. Thus it is believed that human Fen1 accounts for some if not all of the higher molecular weight band showing RNase H activity in the gel renaturation assay. Using the standard RNase H cleavage assay it was confirmed that the expressed Fen1 cleaves the RNA strand of DNA/RNA duplexes. It was found that this enzyme is capable of cleaving both an unmodified DNA/RNA duplex and a gapmer/RNA duplex in which the oligonucleotide (“DNA”) strand of the duplex is a chimeric oligonucleotide with 2′-O-methoxyethyl flanks and a 2′-deoxynucleotide center gap. Thus it is believed that Fen1 may also be suitable for eliciting antisense-mediated cleavage of target RNA.

A human RNase H1 has now been cloned and expressed. The enzyme encoded by this cDNA is inactive against single-stranded RNA, single-stranded DNA and double-stranded DNA. However, this enzyme cleaves the RNA in an RNA/DNA duplex and cleaves the RNA in a duplex comprised of RNA and a chimeric oligonucleotide with 2′ methoxy flanks and a 5-deoxynucleotide center gap. The rate of cleavage of the RNA duplexed with this so-called “deoxy gapmer” was significantly slower than observed with the full RNA/DNA duplex. These properties are consistent with those reported for E. coli RNase H1 (Crooke et al., Biochem. J., 1995, 312, 599-608; Lima, W. F. and Crooke, S. T., Biochemistry, 1997, 36, 390-398). They are also consistent with the properties of a human Type 2 RNase H protein purified from placenta, as the molecular weight (32 kDa) is similar to that reported by Frank et al., Nucleic Acids Res., 1994, 22, 5247-5254) and the enzyme is inhibited by Mn²⁺. Accordingly, we refer to the newly cloned human RNase H as Type 2 RNase H or human RNase H1.

Thus, in accordance with one aspect of the present invention, there are provided isolated polynucleotides which encode RNase H1 polypeptides. By “polynucleotides” it is meant to include any form of RNA or DNA such as mRNA or cDNA or genomic DNA, respectively, obtained by cloning or produced synthetically by well known chemical techniques. DNA may be double- or single-stranded. Single-stranded DNA may comprise the coding or sense strand or the non-coding or antisense strand.

Methods of isolating a polynucleotide of the present invention via cloning techniques are well known. For example, to obtain the cDNA contained in ATCC Deposit No. 98536, primers based on a search of the XREF database were used. An approximately 1-Kb cDNA corresponding to the carboxy terminal portion of the protein was cloned by 3′ RACE. Seven positive clones were isolated by screening a liver cDNA library with this 1-Kb cDNA. The two longest clones were 1698 and 1168 base pairs. They share the same 5′ untranslated region and protein coding sequence but differ in the length of the 3′ UTR. A single reading frame encoding a 286 amino acid protein (calculated mass: 32029.04 Da) was identified. The proposed initiation codon is in agreement with the mammalian translation initiation consensus sequence described by Kozak, M., J. Cell Biol., 1989, 108, 229-241, and is preceded by an in-frame stop codon. Efforts to clone cDNAs with longer 5′ UTRs from both human liver and lymphocyte cDNAs by 5′ RACE failed, indicating that the 1698-base-pair clone was full length.

In a preferred embodiment, the RNase H1 polynucleotide comprises the nucleic acid sequence of the cDNA contained within ATCC Deposit No. 98536 or Genbank accession no. AF039652. The deposit of E. coli DH5α containing a BLUESCRIPT™ plasmid containing a human (Type 2) RNase H1 cDNA was made with the American Type Culture Collection, 12301 Park Lawn Drive, Rockville, Md. 20852, USA, on Sep. 4, 1997 and assigned ATCC Deposit No. 98536. The deposited material is a culture of E. coli DH5α containing a BLUESCRIPT™ plasmid (Stratagene, La Jolla Calif.) that contains the full-length human RNase H1 cDNA. The deposit has been made under the terms of the Budapest Treaty on the international recognition of the deposit of micro-organisms for the purposes of patent procedure. The culture will be released to the public, irrevocably and without restriction to the public upon issuance of this patent. The sequence of the polynucleotide contained in the deposited material and the amino acid sequence of the polypeptide encoded thereby are controlling in the event of any conflict with the sequences provided herein. However, as will be obvious to those of skill in the art upon this disclosure, due to the degeneracy of the genetic code, polynucleotides of the present invention may comprise other nucleic acid sequences encoding the polypeptide and derivatives, variants or active fragments thereof.

Another aspect of the present invention relates to the polypeptides encoded by the polynucleotides of the present invention. A polypeptide of the present invention comprises the deduced amino acid sequence of human RNase H1 provided herein as SEQ ID NO: 1. However, by “polypeptide” it is also meant to include fragments, derivatives and analogs which retain essentially the same biological activity and/or function as human RNase H1. Alternatively, polypeptides of the present invention may retain their ability to bind to an antisense-RNA duplex even though they do not function as active RNase H enzymes in other capacities. In another embodiment, polypeptides of the present invention may retain nuclease activity but without specificity for the RNA portion of an RNA/DNA duplex. Polypeptides of the present invention include recombinant polypeptides, isolated natural polypeptides and synthetic polypeptides, and fragments thereof which retain one or more of the activities described above.

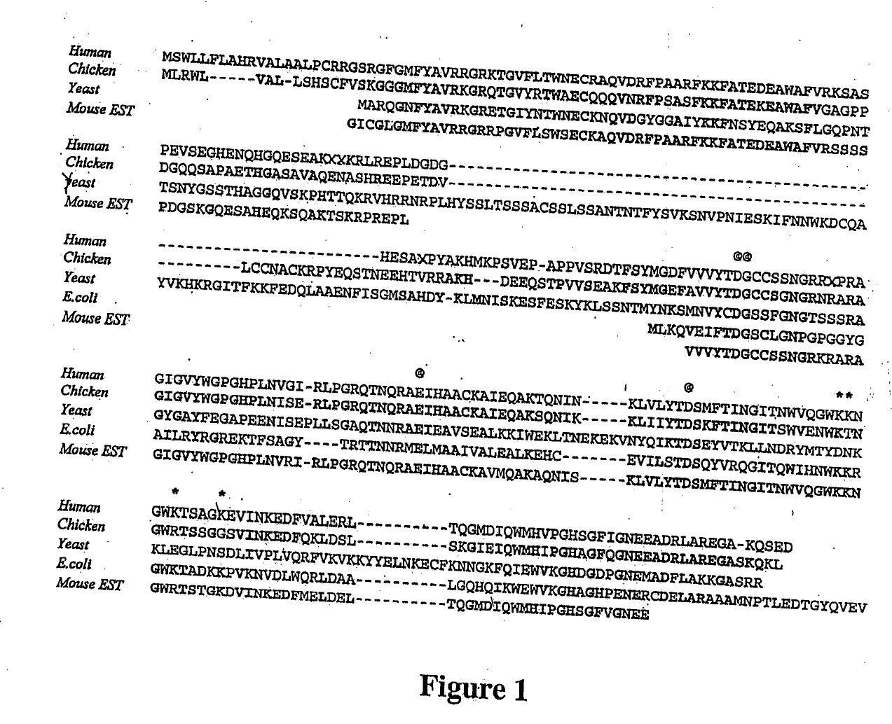

In a preferred embodiment, the polypeptide is prepared recombinantly, most preferably from the culture of E. coli of ATCC Deposit No. 98536. Recombinant human RNase H fused to histidine codons (his-tag; in the present embodiment six histidine codons were used) expressed in E. coli can be conveniently purified to electrophoretic homogeneity by chromatography with Ni-NTA followed by C4 reverse phase HPLC. The polypeptide of SEQ ID NO: 1 is highly homologous to E. coli RNase H, displaying nearly 34% amino acid identity with E. coli RNase H1. FIG. 1 compares a protein sequence deduced from human RNase H1 cDNA (SEQ ID NO: 1) with those of chicken (SEQ ID NO: 2), yeast (SEQ ID NO: 3) and E. coli RNase HI (GenBank accession no. 1786408; SEQ ID NO: 4), as well as an EST deduced mouse RNase H homolog (GenBank accession no. AA389926 and AA518920; SEQ ID NO: 5). The deduced amino acid sequence of human RNase H1 (SEQ ID NO: 1) displays strong homology with yeast (21.8% amino acid identity), chicken (59%), E. coli RNase HI (33.6%) and the mouse EST homolog (84.3%). They are all small proteins (<40 KDa) and their estimated pIs are all 8.7 and greater. Further, the amino acid residues in E. coli RNase HI thought to be involved in the Mg²⁺ binding site, catalytic center and substrate binding region are completely conserved in the cloned human RNase H1 sequence (FIG. 1).

The human RNase H1 is expressed ubiquitously. Northern blot analysis demonstrated that the transcript was abundant in all tissues and cell lines except the MCR-5 line. Northern blot analysis of total RNA from human cell lines and Poly A containing RNA from human tissues using the 1.7 kb full length probe or a 332-nucleotide probe that contained the 5′ UTR and coding region of human RNase H1 cDNA revealed two strongly positive bands with approximately 1.2 and 5.5 kb in length and two less intense bands approximately 1.7 and 4.0 kb in length in most cell lines and tissues. Analysis with the 332-nucleotide probe showed that the 5.5 kb band contained the 5′ UTR and a portion of the coding region, which suggests that this band represents a pre-processed or partially processed transcript, or possibly an alternatively spliced transcript. Intermediate sized bands may represent processing intermediates. The 1.2 kb band represents the full length transcripts. The longer transcripts may be processing intermediates or alternatively spliced transcripts.

RNase H1 is expressed in most cell lines tested; only MRC5, a breast cancer cell line, displayed very low levels of RNase H. However, a variety of other malignant cell lines including those of bladder (T24), breast (T-47D, HS578T), lung (A549), prostate (LNCap, DU145), and myeloid lineage (HL-60), as well as normal endothelial cells (HUVEC), expressed RNase H1. Further, all normal human tissues tested expressed RNase H1. Again, larger transcripts were present as well as the 1.2 kb transcript that appears to be the mature mRNA for RNase H1. Normalization based on G3PDH levels showed that expression was relatively consistent in all of the tissues tested.

The Southern blot analysis of EcoRI digested human and various mammalian vertebrate and yeast genomic DNAs probed with the 1.7 kb probe shows that four EcoRI digestion products of human genomic DNA (2.4, 4.6, 6.0, 8.0 Kb) hybridized with the 1.7 kb probe. The blot re-probed with a 430 nucleotide probe corresponding to the C-terminal portion of the protein showed only one 4.6 kbp EcoRI digestion product hybridized. These data indicate that there is only one gene copy for RNase H1 and that the size of the gene is more than 10 kb. Both the full length and the shorter probe strongly hybridized to one EcoRI digestion product of yeast genomic DNA (about 5 kb in size), indicating a high degree of conservation. These probes also hybridized to the digestion product from monkey, but none of the other tested mammalian genomic DNAs including the mouse which is highly homologous to the human RNase H1 sequence.

A recombinant human RNase H1 (his-tag fusion protein) polypeptide of the present invention was expressed in E. coli and purified by Ni-NTA agarose beads followed by C4 reverse phase column chromatography. A 36 kDa protein copurified with activity measured after renaturation. The presence of the his-tag was confirmed by Western blot analyses with an anti-penta-histidine antibody (Qiagen, Germany).

Renatured recombinant human RNase H1 displayed RNase H activity. Incubation of 10 ng purified renatured RNase H with RNA/DNA substrate for 2 hours resulted in cleavage of 40% of the substrate. The enzyme also cleaved RNA in an oligonucleotide/RNA duplex in which the oligonucleotide was a 2′-methoxy gapmer with a 5-deoxynucleotide gap, but at a much slower rate than the full RNA/DNA substrate. This is consistent with observations with E. coli RNase HI (Lima, W. F. and Crooke, S. T., Biochemistry, 1997, 36, 390-398). It was inactive against single-stranded RNA or double-stranded RNA substrates and was inhibited by Mn²⁺. The molecular weight (˜36 kDa) and inhibition by Mn²⁺ indicate that the cloned enzyme is highly homologous to E. coli RNase HI and has properties consistent with those assigned to Type 2 human RNase H.

The sites of cleavage in the RNA in the full RNA/DNA substrate and the gapmer/RNA duplexes (in which the oligonucleotide gapmer had a 5-deoxynucleotide gap) resulting from the recombinant enzyme were determined. In the full RNA/DNA duplex, the principal site of cleavage was near the middle of the substrate, with evidence of less prominent cleavage sites 3′ to the primary cleavage site. The primary cleavage site for the gapmer/RNA duplex was located across the nucleotide adjacent to the junction of the 2′ methoxy wing and oligodeoxynucleotide gap nearest the 3′ end of the RNA. Thus, the enzyme resulted in a major cleavage site in the center of the RNA/DNA substrate and less prominent cleavages to the 3′ side of the major cleavage site. The shift of its major cleavage site to the nucleotide in apposition to the DNA 2′ methoxy junction of the 2′ methoxy wing at the 5′ end of the chimeric oligonucleotide is consistent with the observations for E. coli RNase HI (Crooke et al., 1995, Biochem. J., 312, 599-608; Lima, W. F. and Crooke, S. T. 1997, Biochemistry 36, 390-398). The fact that the enzyme cleaves at a single site in a 5-deoxy gap duplex indicates that the enzyme has a catalytic region of similar dimensions to that of E. coli RNase HI.

Accordingly, expression of large quantities of a purified human RNase H polypeptide of the present invention is useful in characterizing the activities of a mammalian form of this enzyme. In addition, the polynucleotides and polypeptides of the present invention provide a means for identifying agents which enhance the function of antisense oligonucleotides in human cells and tissues.

For example, a host cell can be genetically engineered to incorporate polynucleotides and express polypeptides of the present invention. Polynucleotides can be introduced into a host cell using any number of well known techniques such as infection, transduction, transfection or transformation. The polynucleotide can be introduced alone or in conjunction with a second polynucleotide encoding a selectable marker. In a preferred embodiment, the host comprises a mammalian cell. Such host cells can then be used not only for production of human RNase H, but also to identify agents which increase or decrease levels of expression or activity of human RNase H in the cell. In these assays, the host cell would be exposed to an agent suspected of altering levels of expression or activity of human RNase H in the cells. The level or activity of human RNase H in the cell would then be determined in the presence and absence of the agent. Assays to determine levels of protein in a cell are well known to those of skill in the art and include, but are not limited to, radioimmunoassays, competitive binding assays, Western blot analysis and enzyme linked immunosorbent assays (ELISAs). Methods of determining increase activity of the enzyme, and in particular increased cleavage of an antisense-mRNA duplex can be performed in accordance with the teachings of the examples below. Agents identified as inducers of the level or activity of this enzyme may be useful in enhancing the efficacy of antisense oligonucleotide therapies.

The present invention also relates to prognostic assays wherein levels of RNase H in a cell type can be used in predicting the efficacy of antisense oligonucleotide therapy in specific target cells. High levels of RNase in a selected cell type are expected to correlate with higher efficacy as compared to lower amounts of RNase in a selected cell type which may result in poor cleavage of the mRNA upon binding with the antisense oligonucleotide. For example, the MRC5 breast cancer cell line displayed very low levels of RNase H as compared to other malignant cell types. Accordingly, in this cell type it may be desired to use antisense compounds which do not depend on RNase H activity for their efficacy. Similarly, oligonucleotides can be screened to identify those which are effective antisense agents by contacting human RNase H1 with an oligonucleotide and measuring binding of the oligonucleotide to the human RNase H1. Methods of determining binding of two molecules are well known in the art. For example, in one embodiment, the oligonucleotide can be radiolabeled and binding of the oligonucleotide to human RNase H1 can be determined by autoradiography. Alternatively, fusion proteins of human RNase H1 with glutathione-S-transferase or small peptide tags can be prepared and immobilized to a solid phase such as beads. Labeled or unlabeled oligonucleotides to be screened for binding to this enzyme can then be incubated with the solid phase. Oligonucleotides which bind to the enzyme immobilized to the solid phase can then be identified either by detection of bound label or by eluting specifically the bound oligonucleotide from the solid phase. Another method involves screening of oligonucleotide libraries for binding partners. Recombinant tagged or labeled human RNase H1 is used to select oligonucleotides from the library which interact with the enzyme. Sequencing of the oligonucleotides leads to identification of those oligonucleotides which will be more effective as antisense agents.

A human RNase H2 has also now been cloned. In accordance with another aspect of the present invention, there are provided isolated polynucleotides which encode human RNase H2 polypeptides having the deduced amino acid sequence of SEQ ID NO: 6. A culture containing this nucleic acid sequence has been deposited as ATCC Deposit No. PTA-2897. “Polynucleotides” is meant to include any form of RNA or DNA such as mRNA or cDNA or genomic DNA, respectively, obtained by cloning or produced synthetically by well known chemical techniques. DNA may be double- or single-stranded. Single-stranded DNA may comprise the coding or sense strand or the non-coding or antisense strand.

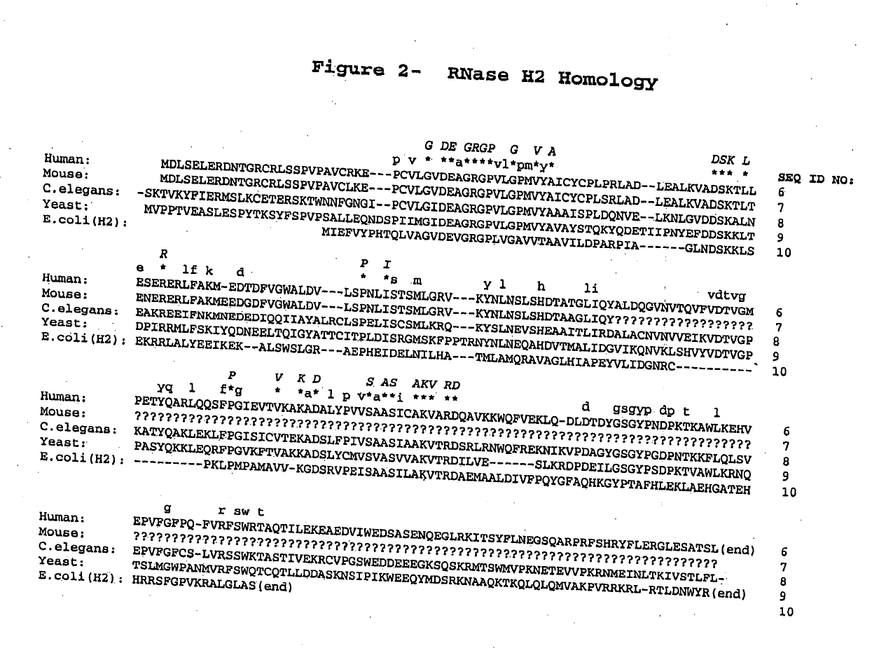

Methods of isolating a polynucleotide of the present invention via cloning techniques are well known. For example, to obtain the cDNA which encodes the RNase H2 polypeptide sequence provided herein as SEQ ID NO: 6, primers based on a search of the XREF database were used. A cDNA corresponding to the carboxy terminal portion of the protein was cloned by 3′ RACE. Positive clones were isolated by screening a human liver cDNA library with this cDNA. A 1131-nucleotide cDNA fragment encoding the full RNase H2 protein sequence was identified and is provided herein as SEQ ID NO: 11. A single reading frame encoding a 299 amino acid protein (calculated mass: 33392.53 Da) was identified (shown in FIG. 2). This polypeptide sequence is provided herein as SEQ ID NO: 6.

In a preferred embodiment, the polynucleotide of the present invention comprises the nucleic acid sequence provided herein as SEQ ID NO: 11. However, as will be obvious to those of skill in the art upon this disclosure, due to the degeneracy of the genetic code, polynucleotides of the present invention may comprise other nucleic acid sequences encoding the polypeptide of SEQ ID NO: 6 and derivatives, variants or active fragments thereof.

The present invention also includes single-stranded and double-stranded antisense compounds that modulate the expression of RNase H. Inhibitors of both RNase H1 and H2 are provided herein, exemplified by single-stranded antisense oligonucleotides which, in some embodiments, are chimeric “gapmer” oligonucleotides comprising 2′-O-methoxyethyl modifications flanking a 2′deoxynucleotide region. In other embodiments the antisense compounds are siRNA compounds, i.e, double-stranded RNA compounds that inhibit RNase H expression. Examples of oligonucleotide design and modifications are described in further detail hereinbelow. It is preferred that antisense inhibitors of RNase H reduce RNase H expression by at least 10%, more preferably by at least 30% compared to untreated or vehicle controls.

Another aspect of the present invention relates to the polypeptides encoded by the polynucleotides of the present invention. In a preferred embodiment, a polypeptide of the present invention comprises the deduced amino acid sequence of human RNase H2 provided in FIG. 2 as SEQ ID NO: 6. However, by “polypeptide” it is also meant to include fragments, mutants, derivatives and analogs of SEQ ID NO: 6 which retain essentially the same biological activity and/or function as human RNase H2. Alternatively, polypeptides of the present invention may retain their ability to bind to an RNA-DNA duplex even though they do not function as active RNase H enzymes in other capacities. In another embodiment, polypeptides of the present invention may retain nuclease activity but without specificity for the RNA portion of an RNA/DNA duplex. Polypeptides of the present invention include recombinant polypeptides, isolated natural polypeptides and synthetic polypeptides, and fragments thereof which retain one or more of the activities described above.

In a preferred embodiment, the polypeptide is prepared recombinantly, most preferably from the cDNA sequence provided herein as SEQ ID NO: 11. Recombinant human RNase H fused to histidine codons (his-tag; in the present embodiment six histidine codons were used) expressed in E. coli can be conveniently purified to electrophoretic homogeneity by chromatography with Ni-NTA followed by C4 reverse phase HPLC.

A recombinant human RNase H2 (his-tag fusion protein) polypeptide of the present invention was expressed in E. coli and purified by Ni-NTA agarose beads followed by C4 reverse phase column chromatography. A 36 kDa protein (approx.) copurified with activity measured after renaturation. The presence of the his-tag was confirmed by Western blot analyses with an anti-penta-histidine antibody (Qiagen, Germany).

Renatured recombinant human RNase H2 displayed a small amount of RNase H activity. Incubation of purified renatured RNase H2 protein with RNA/DNA duplex substrate for 60 minutes resulted in detectable cleavage of the substrate.

Accordingly, expression of large quantities of a purified human RNase H2 polypeptide of the present invention is useful in characterizing the activities of this enzyme as described above. For example, a host cell can be genetically engineered to incorporate polynucleotides and express polypeptides of the present invention. Polynucleotides can be introduced into a host cell using any number of well known techniques such as infection, transduction, transfection or transformation. The polynucleotide can be introduced alone or in conjunction with a second polynucleotide encoding a selectable marker. In a preferred embodiment, the host comprises a mammalian cell. Such host cells can then be used not only for production of human RNase H2, but also to identify agents which increase or decrease levels of expression or activity of human RNase H in the cell. In these assays, the host cell would be exposed to an agent suspected of altering levels of expression or activity of human RNase H in the cells. The level or activity of human RNase H in the cell would then be determined in the presence and absence of the agent. Assays to determine levels of protein in a cell are well known to those of skill in the art and include, but are not limited to, radioimmunoassays, competitive binding assays, Western blot analysis and enzyme linked immunosorbent assays (ELISAs). Methods of determining increased activity of the enzyme, and in particular increased cleavage of an antisense-mRNA duplex can be performed in accordance with the teachings of the following examples. Agents identified as inducers of the level or activity of this enzyme may be useful in enhancing the efficacy of antisense oligonucleotide therapies.

A problem in the study of mammalian RNase H2 until now has been the fact that cloned, expressed and purified human RNase H2 has been only marginally active, or inactive, in the gel renaturation or solution-based assays. While not wishing to be bound by theory, this may be due to the lack of associated proteins necessary for enzyme activity or because the enzyme's conformation is incorrectly reformed when expressed or purified. To overcome this limitation, RNase H2 was immunoprecipitated from HeLa cells using purified antibodies to human RNase H2, then analyzed for activity. Extraction of proteins from the immunoprecipitation beads followed by polyacrylamide gel electrophoresis demonstrated that a number of proteins immunoprecipitated with human RNase H2. To support comparisons between the human RNase H1 and H2, we developed a similar approach for human RNase H1. Both human RNase H1 and H2 were found to be active in the TCA assay after immunoprecipitation. Further, when the enzymes were overexpressed, the activity extracted from the HeLa cells was greater, confirming that for both RNase H1 and RNase H2 the overexpressed enzymes were active.

The cleavage patterns of human RNase H1 and H2 immunoprecipitated from uninfected HeLa cells were compared, using two different RNA-DNA duplex substrates. The enzymes were found to display different cleavage patterns in both substrates. Further, the cleavage pattern observed for immunoprecipitated human RNase H1 was identical to that observed previously with purified RNase H1.

Mutant forms of mammalian RNase H are also useful. As described in the following examples, the roles of the conserved amino acids of the catalytic site and the basic substrate-binding domain (region III), the roles of the dsRNA-binding domain (region I) and the 62 amino acid center region of human RNase H1 (region II) have been explored. Site-directed mutagenesis has here been performed on the three conserved amino acids of the proposed catalytic site of human RNase H1 ([D145N], [E186Q], and [D210N]). In addition, the net positive charge of the basic substrate-binding domain was progressively reduced through alanine substitution of two (RNase H1[K226,227A]) and four (RNase H1 [K226,227,231,236A]) of the lysines within this region. Deletion mutants were also prepared in which either the dsRNA-binding domain of region I (RNase H1[ΔI]), or the central region II (RNase H1[ΔII]) was deleted. Another mutant protein representing the conserved E. coli RNase H1 region was prepared by deleting both region I and II, (RNase H1 [ΔI-II]).

Dominant negative forms of both human RNase H1 and H2 have been designed and made, as described in the following examples. These mutant enzymes have been overexpressed in human cells. Overexpression of wild type and/or dominant negative RNase H is useful in research and for modulating antisense effects of RNase H-dependent antisense oligonucleotides.

The present invention also relates to methods for promoting antisense inhibition of a selected RNA target using mammalian RNase H, or for eliciting cleavage of a selected target via antisense. In the context of this invention, “promoting antisense inhibition” or “promoting inhibition of expression” of a selected RNA target, or of its protein product, means inhibiting expression of the target or enhancing the inhibition of expression of the target. “Enhancing antisense potency” means increasing the ability of an antisense compound to inhibit expression of its RNA target, or increasing the ability of an antisense compound to elicit cleavage of its RNA target. In both cases the effect is intended to be selective for the target to which the antisense compound is targeted (i.e., to which it is specifically hybridizable).

In one preferred embodiment, the mammalian RNase H is a human RNase H. The RNase H may be an RNase H1 or an RNase H2 or may be a larger nuclease with RNase H activity, such as Fen1. In one embodiment of these methods, the mammalian RNase H is present in an enriched amount. In the context of this invention, “enriched” means an amount greater than would naturally be found. RNase H may be present in an enriched amount through, for example, addition of exogenous RNase H, through selection of cells which overexpress RNase H or through manipulation of cells to cause overexpression of RNase H. The exogenously added RNase H may be added in the form of, for example, a cellular or tissue extract (such as HeLa cell extract), a biochemically purified or partially purified preparation of RNase H, or a cloned and expressed RNase H polypeptide. In some embodiments of the methods of the invention, the mammalian RNase H has SEQ ID NO: 1 or 6, or may be another mammalian RNase H such as those described by Cerritelli and Crouch (1998, Genomics 53, 300-307); provided herein as SEQ ID NO: 12 and 14 or by Frank et al. (1998, Biol. Chem. 379, 1407-1412; 1998, Proc. Natl. Acad. Sci. USA, 95, 12872-12877), provided herein as SEQ ID NO: 13 and 15.

The present invention also relates to methods of screening oligonucleotides to identify active antisense oligonucleotides. The oligonucleotides may be present as a library or mixture of oligonucleotides. The methods involve contacting a mammalian RNase H, one or more oligonucleotides and an RNA target under conditions in which an oligonucleotide/RNA duplex is formed. The RNase H may be present in an enriched amount.

Antisense oligonucleotides are frequently used in in vivo experiments and are being evaluated in multiple clinical trials in humans. Experiments in mice were therefore conducted to examine the effects of overexpressing RNase H on potency of DNA-like antisense oligonucleotides in vivo. It was demonstrated that both human RNase H1 and human RNase H2 could be overexpressed in mouse cell lines, and that overexpression of human RNase H1 increased antisense oligonucleotide potency in mouse cells. Overexpression of RNase H2 had no effect on antisense potency.

Mice were then treated with the control and human RNase H1-containing adenovirus. Human RNase H1 was significantly overexpressed in the liver of the animals that were infected with the adenoviruses containing the RNase H1 insert, and this human RNase H1 expressed in mouse liver was shown to be active. To determine if overexpression of human RNase H1 in mouse liver increased antisense potency in vivo, the effects of a well characterized antisense oligonucleotide targeted to mouse Fas were evaluated. The antisense oligonucleotide caused the selective reduction of Fas RNA in mouse liver and overexpression of human RNase H1 increased the potency of the Fas antisense oligonucleotide.

The present invention also relates to prognostic assays wherein levels of RNase H in a cell type can be used in predicting the efficacy of antisense oligonucleotide therapy in specific target cells. High levels of RNase H in a selected cell type are expected to correlate with higher efficacy as compared to lower amounts of RNase H in a selected cell type which may result in poor cleavage of the mRNA upon binding with the antisense oligonucleotide. For example, the HTB-11 neuroblastoma cell line displayed lower levels of RNase H2 than some other malignant cell types. Accordingly, in this cell type it may be desired to use antisense compounds which do not depend on RNase H activity for their efficacy. Similarly, oligonucleotides can be screened to identify those which are effective antisense agents by contacting RNase H with an oligonucleotide and measuring binding of the oligonucleotide to the RNase H. Methods of determining binding of two molecules are well known in the art. For example, in one embodiment, the oligonucleotide can be radiolabeled and binding of the oligonucleotide to human RNase H can be determined by autoradiography. Alternatively, fusion proteins of human RNase H with glutathione-S-transferase or small peptide tags can be prepared and immobilized to a solid phase such as beads. Labeled or unlabeled oligonucleotides to be screened for binding to this enzyme can then be incubated with the solid phase. Oligonucleotides which bind to the enzyme immobilized to the solid phase can then be identified either by detection of bound label or by eluting specifically the bound oligonucleotide from the solid phase. Another method involves screening of oligonucleotide libraries for binding partners. Recombinant tagged or labeled human RNase H is used to select oligonucleotides from the library which interact with the enzyme. Sequencing of the oligonucleotides leads to identification of those oligonucleotides which will be more effective as antisense agents.

The modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the target. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of the target, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are hereinbelow identified as preferred embodiments of the invention. The target sites to which these preferred sequences are complementary are hereinbelow referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 80 nucleobases (i.e. from about 8 to about 80 linked nucleosides). Particularly preferred antisense compounds are antisense oligonucleotides, even more preferably those comprising from about 13 to about 50 nucleobases, and even more preferably about 15 to about 30 or from 19 to 24 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. Both single-stranded and fully or partially double-stranded antisense compounds (the latter comprehends siRNA or RNAi compounds) are included.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′-, 3′- or 5′-hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙNH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methylene (—CH₂—)ₙ group bridging the 2′ oxygen atom and the 3′ or 4′ carbon atom wherein n is 1 or 2. In the case of an ethylene group in this position, the term ENA™ is used (Singh et al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10 C), stability towards 3′-exonucleolytic degradation and good solubility properties. LNA's are commercially available from ProLigo (Paris, France and Boulder, Colo., USA).

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C/C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2EC (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference. Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-o-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference. It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Oligonucleotides, particularly chimeric oligonucleotides, designed to elicit target cleavage by RNase H, thus are generally more potent than oligonucleotides of the same base sequence which are not so optimized. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

RNase H, by definition, cleaves the RNA strand of an RNA-DNA duplex. In exploiting RNase H for antisense technology, the DNA portion of the duplex is generally an antisense oligonucleotide. Because native DNA oligonucleotides (2′ deoxy oligonucleotides with phosphodiester linkages) are relatively unstable in cells due to poor nuclease resistance, modified oligonucleotides are preferred for antisense. For example, oligodeoxynucleotides with phosphorothioate backbone linkages are often used. This is an example of a DNA-like oligonucleotide which is able to elicit RNase H cleavage of its complementary target RNA. Nucleic acid helices can adopt more than one type of structure, most commonly the A-and B-forms. It is believed that, in general, oligonucleotides which have B-form-like conformational geometry are “DNA-like” and will be able to elicit RNase H upon duplexation with an RNA target. Furthermore, oligonucleotides which contain a “DNA-like” region of B-form-like conformational geometry are also believed to be able to elicit RNase H upon duplexation with an RNA target.

The nucleotides for this B-form portion are selected to specifically include ribo-pentofuranosyl and arabino-pentofuranosyl nucleotides. 2′-Deoxy-erythro-pentofuranosyl nucleotides also have B-form geometry and elicit RNase H activity. While not specifically excluded, if 2′-deoxy-erythro-pentofuranosyl nucleotides are included in the B-form portion of an oligonucleotide of the invention, such 2′-deoxy-erythro-pentofuranosyl nucleotides preferably does not constitute the totality of the nucleotides of that B-form portion of the oligonucleotide, but should be used in conjunction with ribonucleotides or an arabino nucleotides. As used herein, B-form geometry is inclusive of both C2′-endo and O4′-endo pucker, and the ribo and arabino nucleotides selected for inclusion in the oligonucleotide B-form portion are selected to be those nucleotides having C2′-endo conformation or those nucleotides having O4′-endo conformation. This is consistent with Berger, et. al., Nucleic Acids Research, 1998, 26, 2473-2480, who pointed out that in considering the furanose conformations in which nucleosides and nucleotides reside, B-form consideration should also be given to a O4′-endo pucker contribution.

Preferred for use as the B-form nucleotides for eliciting RNase H are ribonucleotides having 2′-deoxy-2′-S-methyl, 2′-deoxy-2′-methyl, 2′-deoxy-2′-amino, 2′-deoxy-2′-mono or dialkyl substituted amino, 2′-deoxy-2′-fluoromethyl, 2′-deoxy-2′-difluoromethyl, 2′-deoxy-2′-trifluoromethyl, 2′-deoxy-2′-methylene, 2′-deoxy-2′-fluoromethylene, 2′-deoxy-2′-difluoromethylene, 2′-deoxy-2′-ethyl, 2′-deoxy-2′-ethylene and 2′-deoxy-2′-acetylene. These nucleotides can alternately be described as 2′-SCH₃ ribonucleotide, 2′-CH₃ ribonucleotide, 2′-NH₂ ribonucleotide 2′-NH(C₁-C₂ alkyl) ribonucleotide, 2′-N(C₁-C₂ alkyl)₂ ribonucleotide, 2′-CH₂F ribonucleotide, 2′-CHF₂ ribonucleotide, 2′-CF₃ ribonucleotide, 2′═CH₂ ribonucleotide, 2′=CHF ribonucleotide, 2′═CF₂ ribonucleotide, 2′-C₂H₅ ribonucleotide, 2′-CH═CH₂ ribonucleotide, 2′-C/CH ribonucleotide. A further useful ribonucleotide is one having a ring located on the ribose ring in a cage-like structure including 3′,O,4═—C-methyleneribonucleotides. Such cage-like structures will physically fix the ribose ring in the desired conformation.

Additionally, preferred for use as the B-form nucleotides for eliciting RNase H are arabino nucleotides having 2′-deoxy-2′-cyano, 2′-deoxy-2′-fluoro, 2′-deoxy-2′-chloro, 2′-deoxy-2′-bromo, 2′-deoxy-2′-azido, 2′-methoxy and the unmodified arabino nucleotide (that includes a 2′-OH projecting upwards towards the base of the nucleotide). These arabino nucleotides can alternately be described as 2′-CN arabino nucleotide, 2′-F arabino nucleotide, 2′-Cl arabino nucleotide, 2′-Br arabino nucleotide, 2′-N₃ arabino nucleotide, 2′-O—CH₃ arabino nucleotide and arabino nucleotide.

Such nucleotides are linked together via phosphorothioate, phosphorodithioate, boranophosphate or phosphodiester linkages. particularly preferred is the phosphorothioate linkage.

Illustrative of the B-form nucleotides for use in the invention is a 2′-S-methyl (2′-SMe) nucleotide that resides in C2′ endo conformation. It has been compared by molecular modeling to a 2′-O-methyl (2′-OMe)nucleotide that resides in a C3′ endo conformation.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Any single-stranded antisense sequence can be prepared as a double-stranded compound. The duplex has an antisense strand that is substantially complementary to the target sequence, and a sense strand that is substantially complementary to the antisense strand. The duplex may be unimolecular or bimolecular, i.e., the sense and antisense strands may be part of the same molecule (which forms a hairpin or other self structure) or two (or even more) separate molecules. The nucleobase sequence of the antisense strand of the duplex may comprise at least a portion of an oligonucleotide in one of the tables hereinbelow, or another sequence determined empirically (e.g., by a gene walk) or otherwise. The ends of the strands may be modified by the addition of one or more natural or modified nucleobases to form an overhang. The sense strand of the duplex is then designed and synthesized as the complement of the antisense strand and may also contain modifications or additions to either terminus. For example, in one embodiment, both strands of the dsRNA duplex would be complementary over the central nucleobases, each having overhangs at one or both termini. For example, a duplex comprising an antisense strand having the sequence CGAGAGGCGGACGGGACCG and having a two-nucleobase overhang of deoxythymidine(dT) on the 3′ end of each strand would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement (sense strand) |

The one or more nucleobases forming the single-stranded overhang(s) may be dT as shown or may be another modified or unmodified nucleobase which may be complementary to the target (in the case of the antisense strand) or not. The duplex shown above is often referred to as a “canonical” duplex, a 19-base double-stranded RNA region with a dTdT overhang at each 3′ end. Elbashir et al., Nature, 2001, 411, 494-498. However, the duplex need not be of similar structure at both ends, i.e., it may be blunt on one end and have a single-stranded overhang on the other end, or may have overhangs of different lengths.

As another example, a duplex with blunt ends (no single stranded overhang) comprising an antisense strand having the sequence CGAGAGGCGGACGGGACCG may have the following structure:

| | |
|---|---|
| cgagaggcggacgggaccg | Antisense Strand |
| \|\|\|\|\|\|\|\|\|\|\|\|\|\|\|\|\|\|\| | |
| gctctccgcctgccctggc | Complement (Sense strand) |

Such double stranded oligonucleotide moieties have been shown in the art to modulate target expression and regulate translation as well as RNA processing via an antisense mechanism. In some embodiments both strands are RNA. Moreover, the double-stranded moieties may be subject to chemical modifications of one or both strands (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200) and as described below. For example, such double-stranded moieties have been shown to inhibit the target by the classical hybridization of antisense strand of the duplex to the target, thereby triggering enzymatic degradation of the target (Tijsterman et al., Science, 2002, 295, 694-697). Both RNase H-based antisense (usually using single-stranded compounds) and siRNA (usually using double-stranded compounds) are antisense mechanisms, typically resulting in loss of target RNA function. Optimized siRNA and RNase H-dependent oligomeric compounds behave similarly in terms of potency, maximal effects, specificity and duration of action, and efficiency. Moreover it has been shown that in general, activity of dsRNA constructs correlated with the activity of RNase H-dependent single-stranded antisense compounds targeted to the same site. One major exception is that RNase H-dependent antisense compounds were generally active against target sites in pre-mRNA whereas siRNAs were not. Vickers et al., 2003, J. Biol. Chem. 278, 7108.

The following nonlimiting examples are provided to further illustrate the present invention.

### EXAMPLES

### Example 1

### Rapid Amplification of 5′-cDNA End (5′-RACE) and 3′-cDNA End (3′-RACE) of Human RNase H1

An internet search of the XREF database in the National Center of Biotechnology Information (NCBI) yielded a 361 base pair (bp) human expressed sequence tag (EST, GenBank accession no. H28861), homologous to yeast RNase H(RNH1) protein sequence tag (EST, GenBank accession no. Q04740) and its chicken homologue (accession no. D26340). Three sets of oligonucleotide primers encoding the human RNase H EST sequence were synthesized. The sense primers were ACGCTGGCCGGGAGTCGAAATGCTTC (H1: SEQ ID NO: 16), CTGTTCCTGGCCCACAGAGTCGCCTTGG (H3: SEQ ID NO: 17) and GGTCTTTCTGACCTGGAATGAGTGCAGAG (H5: SEQ ID NO: 18). The antisense primers were CTTGCCTGGTTTCGCCCTCCGATTCTTGT (H2: SEQ ID NO: 19), TTGATTTTCATGCCCTTCTGAAACTTCCG (H4; SEQ ID NO: 20) and CCTCATCCTCTATGGCAAACTTCTTAAATCTGGC (H6; SEQ ID NO: 21). The human RNase H 3′ and 5′ cDNAs derived from the EST sequence were amplified by polymerase chain reaction (PCR), using human liver or leukemia (lymphoblastic Molt-4) cell line Marathon ready cDNA as templates, H1 or H3/AP1 as well as H4 or H6/AP2 as primers (Clontech, Palo Alto, Calif.). The fragments were subjected to agarose gel electrophoresis and transferred to nitrocellulose membrane (Bio-Rad, Hercules Calif.) for confirmation by Southern blot, using ³²P-labeled H2 and H1 probes (for 3′ and 5′ RACE products, respectively, in accordance with procedures described by Ausubel et al., Current Protocols in Molecular Biology, Wiley and Sons, New York, N.Y., 1988. The confirmed fragments were excised from the agarose gel and purified by gel extraction (Qiagen, Germany), then subcloned into Zero-blunt vector (Invitrogen, Carlsbad, Calif.) and subjected to DNA sequencing.

### Example 2

### Screening of the cDNA Library, DNA Sequencing and Sequence Analysis of Human RNase H1

A human liver cDNA lambda phage Uni-ZAP library (Stratagene, La Jolla, Calif.) was screened using the RACE products as specific probes. The positive cDNA clones were excised into the pBluescript phagemid (Stratagene, La Jolla Calif.) from lambda phage and subjected to DNA sequencing with an automatic DNA sequencer (Applied Biosystems, Foster City, Calif.) by Retrogen Inc. (San Diego, Calif.). The overlapping sequences were aligned and combined by the assembling programs of MacDNASIS v3.0 (Hitachi Software Engineering America, South San Francisco, Calif.). Protein structure and subsequence analysis were performed by the program of MacVector 6.0 (Oxford Molecular Group Inc., Campbell, Calif.). A homology search was performed on the NCBI database by internet E-mail.

### Example 3

### Northern Blot and Southern Blot Analysis

Total RNA from different human cell lines (ATCC, Rockville, Md.) was prepared and subjected to formaldehyde agarose gel electrophoresis in accordance with procedures described by Ausubel et al., Current Protocols in Molecular Biology, Wiley and Sons, New York, N.Y., 1988, and transferred to nitrocellulose membrane (Bio-Rad, Hercules Calif.). Northern blot hybridization was carried out in QuickHyb buffer (Stratagene, La Jolla, Calif.) with ³²P-labeled probe of full length RNase H cDNA clone or primer H1/H2 PCR-generated 322-base N-terminal RNase H cDNA fragment at 68° for 2 hours. The membranes were washed twice with 0.1% SSC/0.1% SDS for 30 minutes and subjected to auto-radiography. Southern blot analysis was carried out in 1× pre-hybridization/hybridization buffer (BRL, Gaithersburg, Md.) with a ³²P-labeled 430 bp C-terminal restriction enzyme PstI/PvuII fragment or 1.7 kb full length cDNA probe at 60° C. for 18 hours. The membranes were washed twice with 0.1% SSC/0.1% SDS at 60° C. for 30 minutes, and subjected to autoradiography.

### Example 4

### Expression and Purification of the Cloned Human RNase H1 Protein

The cDNA fragment coding the full RNase H protein sequence was amplified by PCR using 2 primers, one of which contains restriction enzyme NdeI site adapter and six histidine (his-tag) codons and 22 bp protein N terminal coding sequence. The fragment was cloned into expression vector pET17b (Novagen, Madison, Wis.) and confirmed by DNA sequencing. The plasmid was transfected into E. coli BL21(DE3) (Novagen, Madison, Wis.). The bacteria were grown in M9ZB medium at 32° C. and harvested when the OD₆₀₀ of the culture reached 0.8, in accordance with procedures described by Ausubel et al., Current Protocols in Molecular Biology, Wiley and Sons, New York, N.Y., 1988. Cells were lysed in 8M urea solution and recombinant protein was partially purified with Ni-NTA agarose (Qiagen, Germany). Further purification was performed with C4 reverse phase chromatography (Beckman, System Gold, Fullerton, Calif.) with 0.1% TFA water and 0.1% TFA acetonitrile gradient of 0% to 80% in 40 minutes as described by Deutscher, M. P., Guide to Protein Purification, Methods in Enzymology 182, Academic Press, New York, N.Y., 1990. The recombinant proteins and control samples were collected, lyophilized and subjected to 12% SDS-PAGE as described by Ausubel et al. (1988) Current Protocols in Molecular Biology, Wiley and Sons, New York, N.Y. The purified protein and control samples were resuspended in 6 M urea solution containing 20 mM Tris HCl, pH 7.4, 400 mM NaCl, 20% glycerol, 0.2 mM PMSF, 5 mM DTT, 10 μg/ml aprotinin and leupeptin, and refolded by dialysis with decreasing urea concentration from 6 M to 0.5 M as well as DTT concentration from 5 mM to 0.5 mM as described by Deutscher, M. P., Guide to Protein Purification, Methods in Enzymology 182, Academic Press, New York, N.Y., 1990. The refolded proteins were concentrated (10 fold) by Centricon (Amicon, Danvers, Mass.) and subjected to RNase H activity assay.

### Example 5

### RNase H Activity Assay

³²P-end-labeled 17-mer RNA, GGGCGCCGUCGGUGUGG (SEQ ID NO: 22) described by Lima, W. F. and Crooke, S. T., Biochemistry, 1997 36, 390-398, was gel-purified as described by Ausubel et al., Current Protocols in Molecular Biology, Wiley and Sons, New York, N.Y., 1988 and annealed with a tenfold excess of its complementary 17-mer oligodeoxynucleotide or a 5-base DNA gapmer, i.e., a 17mer oligonucleotide which has a central portion of five deoxynucleotides (the “gap”) flanked on both sides by six 2′-methoxynucleotides. Annealing was done in 10 mM Tris HCl, pH 8.0, 10 mM MgCl, 50 mM KCl and 0.1 mM DTT to form one of three different substrates: (a) single strand (ss) RNA probe, (b) full RNA/DNA duplex and (c) RNA/DNA gapmer duplex. Each of these substrates was incubated with human RNase H1 protein samples at 37° C. for 5 minutes to 2 hours at the same conditions used in the annealing procedure and the reactions were terminated by adding EDTA in accordance with procedures described by Lima, W. F. and Crooke, S. T., Biochemistry, 1997, 36, 390-398. The reaction mixtures were precipitated with TCA centrifugation and the supernatant was measured by liquid scintillation counting (Beckman LS6000IC, Fullerton, Calif.). An aliquot of the reaction mixture was also subjected to denaturing (8 M urea) acrylamide gel electrophoresis in accordance with procedures described by Lima, W. F. and Crooke, S. T., Biochemistry, 1997, 36, 390-398 and Ausubel et al., Current Protocols in Molecular Biology, Wiley and Sons, New York, N.Y., 1988.

### Example 6

### Cloning Human RNase H2 by Rapid Amplification of 5′-cDNA End (5′-RACE) and 3′-cDNA End (3′-RACE)

An internet search of the XREF database in the National Center of Biotechnology Information (NCBI) yielded 2 overlapping human expressed sequence tags (ESTs), GenBank accession numbers WO5602 and H43540, homologous to yeast RNase HII (RNH2) protein sequence (GenBank accession number Z71348; SEQ ID NO: 9 shown in FIG. 2), and its C. elegans homologue (accession number Z66524, of which amino acids 396-702 of gene TI3H5.2 correspond to SEQ ID NO: 8 shown in FIG. 2). Three sets of oligonucleotide primers hybridizable to one or both of the human RNase H2 EST sequences were synthesized. The sense primers were AGCAGGCGCCGCTTCGAGGC (H1A; SEQ ID NO: 23), CCCGCTCCTGCAGTATTAGTTCTTGC (H1B; SEQ ID NO: 24) and TTGCAGCTGGTGGTGGCGGCTGAGG (H1C; SEQ ID NO: 25). The antisense primers were TCCAATAGGGTCTTTGAGTCTGCCAC (H1D; SEQ ID NO: 26), CACTTTCAGCGCCTCCAGATCTGCC (H1E; SEQ ID NO: 27) and GCGAGGCAGGGGACAATAACAGATGG (H1F; SEQ ID NO: 28). The human RNase H2 3′ cDNA derived from the EST sequence were amplified by polymerase chain reaction (PCR), using human liver Marathon ready cDNA (Clontech, Palo Alto, Calif.) as templates and H1A or H1B/AP1 (for first run PCR) as well as H1B or H1C/AP2 (for second run PCR) as primers. AP1 and AP2 are primers designed to hybridize to the Marathon ready cDNA linkers (linking cDNA insert to vector). The fragments were subjected to agarose gel electrophoresis and transferred to nitrocellulose membrane (Bio-Rad, Hercules Calif.) for confirmation by Southern blot, using a ³²P-labeled H1E probe (for 3′ RACE). The confirmed fragments were excised from the agarose gel and purified by gel extraction (Qiagen, Germany), then subcloned into a zero-blunt vector (Invitrogen, Carlsbad, Calif.) and subjected to DNA sequencing. The human RNase H2 5′ cDNA from the EST sequence was similarly amplified by 5′ RACE. The overlapping sequences were aligned and combined by the assembling programs of MacDNASIS v. 3.0 (Hitachi Software Engineering Co., America, Ltd.). The full length human RNase H2 open reading frame nucleotide sequence obtained is provided herein as SEQ ID NO: 11. Protein structure and analysis were performed by the program MacVector v6.0 (Oxford Molecular Group, UK). The 299-amino acid human RNase H2 protein sequence encoded by the open reading frame is provided herein as SEQ ID NO: 6.

### Example 7

### Screening of the cDNA Library and DNA Sequencing of Human RNase H2 cDNA

A human liver cDNA lambda phage Uni-ZAP library (Stratagene, La Jolla, Calif.) was screened using the 3′ RACE products as specific probes. The positive cDNA clones were excised into pBluescript phagemid from lambda phage and subjected to DNA sequencing. Sequencing of the positive clones was performed with an automatic DNA sequencer by Retrogen Inc. (San Diego, Calif.).

### Example 8

### Northern Hybridization

Total RNA was isolated from different human cell lines (ATCC, Rockville, Md.) using the guanidine isothiocyanate method (21). Ten μg of total RNA was separated on a 1.2% agarose/formaldehyde gel and transferred to Hybond-N+ (Amersham, Arlington Heights, Ill.) followed by fixing using UV crosslinker (Strategene, La Jolla, Calif.). The premade multiple tissue Northern Blot membranes were also purchased from Clontech (Palo Alto, Calif.). To detect RNase H2 mRNA, hybridization was performed by using ³²P-labeled human RNase H II cDNA in Quik-Hyb buffer (Strategene, La Jolla, Calif.) at 68 EC for 2 hours. After hybridization, membranes were washed in a final stringency of 0.1×SSC/0.1% SDS at 60 EC for 30 minutes and subjected to auto-radiography.

RNase H2 was detected in all human tissues examined (heart, brain, placenta, lung, liver, muscle, kidney and pancreas). RNase H2 was also detected in all human cell lines tested (A549, Jurkat, NHDF, Sy5y, T24, MCF7, IMR32, HTB11, HUVEC, T47D, LnCAP, MRC5 and HL60) with the possible exception of NHDF for which presence or absence of a band was difficult to determine in this experiment. MCF7 cells appeared to have relatively high levels and HTB11 and HUVEC cells appeared to have relatively low levels compared to most cell lines.

### Example 9

### Expression and Purification of the Cloned RNase H2 Protein

The cDNA fragment encoding the full RNase H2 protein sequence was amplified by PCR using 2 primers, one of which contains a restriction enzyme NdeI site adapter and six histidine (his-tag) codons and a 22-base pair protein N terminal coding sequence, the other contains an XhoI site and 24 bp protern C-terminal coding sequence including stop codon. The fragment was cloned into expression vector pET17b (Novagen, Madison, Wis.) and confirmed by DNA sequencing. The plasmid was transfected into E. coli BL21(DE3) (Novagen, Madison, Wis.). The bacteria were grown in LB medium at 37EC and harvested when the OD₆₀₀ of the culture reached 0.8, in accordance with procedures described by Ausubel et al., (Current Protocols in Molecular Biology, Wiley and Sons, New York, N.Y., 1988). Cells were lysed in 8M urea solution and recombinant protein was partially purified with Ni-NTA agarose (Qiagen, Germany). Further purification was performed with C4 reverse phase chromatography (Beckman, System Gold, Fullerton, Calif.) with 0.1% TFA water and 0.1% TFA acetonitrile gradient of 0% to 80% in 40 minutes as described by Deutscher, M. P., (Guide to Protein Purification, Methods in Enzymology 182, Academic Press, New York, N.Y., 1990). The recombinant proteins and control samples were collected, lyophilized and subjected to 12% SDS-PAGE as described by Ausubel et al. (1988) (Current Protocols in Molecular Biology, Wiley and Sons, New York, N.Y.). The purified protein and control samples were resuspended in 6 M urea solution containing 20 mM Tris HCl, pH 7.4, 400 mM NaCl, 20% glycerol, 0.2 mM PMSF, 40 mM DTT, 10 μg/ml aprotinin and leupeptin, and refolded by dialysis with decreasing urea concentration from 6 M to 0.5 M as well as DTT concentration from 40 mM to 0.5 mM as described by Deutscher, M. P., (Guide to Protein Purification, Methods in Enzymology 182, Academic Press, New York, N.Y., 1990). The refolded proteins were concentrated (10 fold) by Centricon (Amicon, Danvers, Mass.) and subjected to an RNase H activity assay as described in example 5. After 60 minutes, cleavage of the substrate RNA/DNA duplex was detectable.

### Example 10

### Characterization of Cloned Human RNase H2

The calculated molecular weight, estimated pI and amino acid composition of the cloned RNase H2 are shown in Table 1. The deduced amino acid sequence of the RNase H2 is provided herein as SEQ ID NO: 6.

TABLE 1
Characteristics of cloned human RNase H2

| | Human RNase | E. Coli | Yeast RNase |
|---|---|---|---|
| | HII | RNaseHII | HII |
| Calculated | 33392.53* | 21524.39 | 33923.36 |
| Molecular | | | |
| Weight | | | |
| Estimated pI | 4.94* | 7.48 | 9.08 |

| Amino acid | | | | | | | |
|---|---|---|---|---|---|---|---|
| composition | | No. | Percent | No. | Percent | No. | Percent |
| Nonpolar | A | 25 | 8.36 | 28 | 14.14 | 19 | 6.33 |
| | V | 26 | 8.70 | 16 | 8.08 | 24 | 8.00 |
| | L | 31 | 10.37 | 21 | 10.61 | 23 | 7.67 |
| | I | 8 | 2.68 | 11 | 5.56 | 16 | 5.33 |
| | P | 16 | 5.35 | 13 | 6.57 | 22 | 7.33 |
| | M | 5 | 1.67 | 6 | 3.03 | 11 | 3.67 |
| | F | 11 | 3.68 | 5 | 2.51 | 7 | 2.33 |
| | W | 5 | 1.67 | 1 | 0.51 | 4 | 1.33 |
| Polar | G | 17 | 5.69 | 14 | 7.07 | 12 | 4.00 |
| | S | 23 | 7.69 | 9 | 4.55 | 24 | 8.00 |
| | T | 16 | 5.35 | 6 | 3.03 | 19 | 6.33 |
| | C* | 6 | 2.01 | 1 | 0.51 | 3 | 1.00 |
| | Y | 10 | 3.34 | 5 | 2.53 | 14 | 4.67 |
| | N | 8 | 2.68 | 3 | 1.52 | 11 | 3.67 |
| | Q | 13 | 4.35 | 4 | 2.02 | 17 | 5.67 |
| Acidic | D | 19 | 6.35 | 8 | 4.04 | 19 | 6.33 |
| | E | 23 | 7.39 | 15 | 7.58 | 14 | 4.67 |
| Basic | K | 16 | 5.35 | 11 | 5.56 | 24 | 8.00 |
| | R | 18 | 6.02 | 12 | 6.06 | 15 | 5.00 |
| | H | 3 | 1.00 | 9 | 4.55 | 2 | 0.67 |

### Example 11

### Antisense Oligonucleotide Inhibition of RNase H2 Expression

A series of antisense oligonucleotides were targeted to the human RNase H2 polynucleotide sequence (SEQ ID NO: 11). These compounds were all 2′-O-methoxyethyl “gapmers” with an 8-nucleotide deoxy gap and a phosphorothioate backbone. Cytosine residues are 5-methyl cytosines. These compounds are shown in Table 2. The 2′-O-methoxyethyl (2′MOE) nucleotides are shown in bold.

TABLE 2

Antisense oligonucleotides
targeted to human RNase H2

| | | | | |
|---|---|---|---|---|
| ISIS | NUCLEOTIDE SEQUENCE | SEQ | TARGET | TARGET |
| NO. | (5′ -> 3′) | ID NO: | SITE¹ | REGION |

| | | | | |
|---|---|---|---|---|
| 21946 | CGCCTCAGCCGCCACCACCA | 29 | 28 | 5′ UTR |
| 21947 | CACAGGCGAACTCAGGCGAC | 30 | 90 | Coding |
| 21948 | GGACAATAACAGATGGCGTA | 31 | 188 | Coding |
| 21949 | CCCGCTCGCTCTCCAATAGG | 32 | 259 | Coding |
| 21950 | CCCAGCCGACAAAGTCCGTG | 33 | 304 | Coding |
| 21951 | CGGTGTCCACGAATACCTGG | 34 | 457 | Coding |
| 21952 | CGCGCCTGGTATGTCTCTGG | 35 | 485 | Coding |
| 21953 | GGTAGAGGGCATCTGCTTTG | 36 | 547 | Coding |
| 21954 | CCACCTTGGCACAGATGCTG | 37 | 583 | Coding |
| 21955 | CAGTTTCTCCACGAATTGCC | 38 | 627 | Coding |
| 21956 | TTTTGTCTTGGGATCATTGG | 39 | 681 | Coding |
| 21957 | AGCTGAACCGGACAAACTGG | 40 | 742 | Coding |
| 21958 | CCTCTTTCTCCAGGATGGTC | 41 | 775 | Coding |
| 21959 | ACTCCAGGCCGCGTTCCAGG | 42 | 913 | Coding |
| 21960 | CCTACGTGTGGTTCTCCTTA | 43 | 1003 | 3′ UTR |
| 21961 | GCACACTCCCACCTTGCTTC | 44 | 1041 | 3′ UTR |
| 21962 | CAAAAGGAAGTAGCTGGACC | 45 | 1071 | 3′ UTR |

¹Location (position) of the 5′-most nucleotide of the oligonucleotide target site on the RNase H2 target nucleotide sequence (SEQ ID NO: 11).

The oligonucleotides shown in Table 2 were tested by Northern blot analysis for their ability to inhibit expression of human RNase H2. Results are expressed in Table 3.

TABLE 3
Antisense inhibition of RNase H2 expression

| ISIS | | | SEQ |
|---|---|---|---|
| NO. | % of control | % inhibition | ID NO: |
| 21946 | 50 | 50 | 29 |
| 21947 | 37 | 63 | 30 |
| 21948 | 38 | 62 | 31 |
| 21949 | 18 | 82 | 32 |
| 21950 | 32 | 68 | 33 |
| 21951 | 26 | 74 | 34 |
| 21952 | 11 | 89 | 35 |
| 21953 | 41 | 59 | 36 |
| 21954 | 23 | 77 | 37 |
| 21955 | 67 | 33 | 38 |
| 21956 | 37 | 63 | 39 |
| 21957 | 32 | 68 | 40 |
| 21958 | 62 | 38 | 41 |
| 21959 | 18 | 82 | 42 |
| 21960 | 8 | 92 | 43 |
| 21961 | 93 | 7 | 44 |
| 21962 | 63 | 37 | 45 |

ISIS 21946, 21947, 21948, 21949, 21950, 21951, 21952, 21953, 21954, 21956, 21957, 21959 and 21960 gave at least 50% inhibition of human RNase H2 expression in this assay. Dose response curves for the two most active oligonucleotides in this experiment (ISIS 21952 and 21960) showed a 60% reduction of expression using either oligonucleotide at the lowest dose tested (50 nM) and approximately 70% reduction (ISIS 21952) and >80% reduction (ISIS 21960) at a concentration of 200 nM in A549 cells.

Additional oligonucleotides were targeted to human RNase H2 (SEQ ID NO: 11). These are shown in Table 4. These compounds are either 2′-O-methoxyethyl “gapmers” with a phosphorothioate backbone or uniform 2′-O-methoxyethyls with a phosphorothiate backbone. Cytosine residues are 5-methyl cytosines. The 2′-O-methoxyethyl (2′MOE) nucleotides are shown in bold.

TABLE 4

Antisense oligonucleotides
targeted to human RNase H2

| ISIS | NUCLEOTIDE SEQUENCE | SEQ | TARGET | TARGET |
|---|---|---|---|---|
| NO. | (5′ -> 3′) | ID NO: | SITE¹ | REGION |
| 113435 | AAACAATTTTAATGTCTGGG | 46 | 984 | 3′ UTR |
| 113436 | AATTTTAATGTCTGGGTTGG | 47 | 980 | 3′ UTR |
| 113437 | CCTTAAACAATTTTAATGTC | 48 | 988 | 3′ UTR |
| 113449 | AAACAATTTTAATGTCTGGG | 46 | 984 | 3′ UTR |
| 113450 | AATTTTAATGTCTGGGTTGG | 47 | 980 | 3′ UTR |
| 113451 | CCTTAAACAATTTTAATGTC | 48 | 988 | 3′ UTR |

### Example 12

### Construction of Mutant RNase H1 Proteins

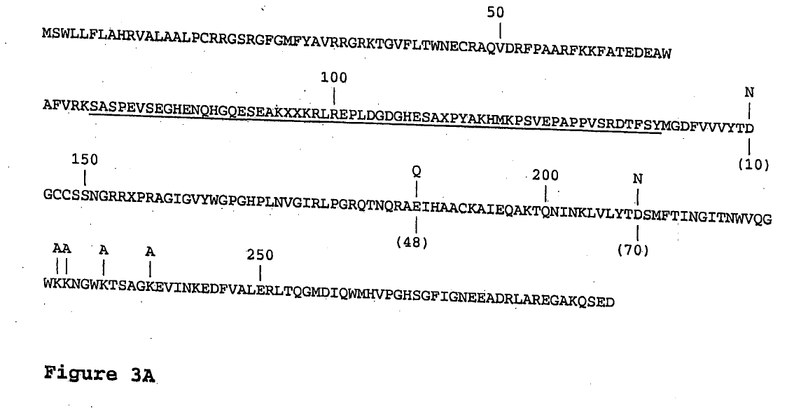

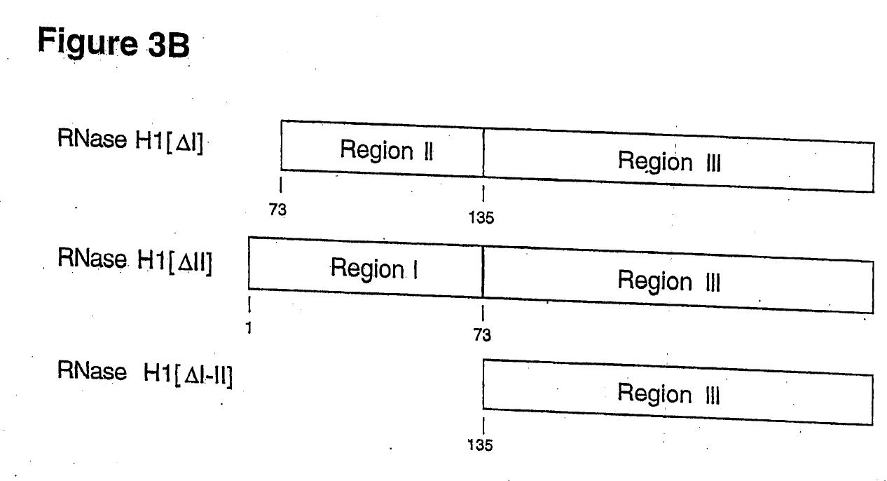

The mutagenesis of human RNase H1 was performed using a PCR-based technique derived from Landt, et al. (1990) Gene 96, 125-128. Briefly, two separate PCR reactions were performed using a set of site-directed mutagenic primers and two vector-specific primers. Wu, H., Lima, W. F., and Crooke, S. T., 1998, Antisense Nucleic Acid Drug Dev. 8, 53-61. Approximately 1 μg of human RNase H1 cDNA was used as the template for the first round of amplification of both the amino- and carboxy-terminal portions of the cDNA corresponding to the mutant site. The fragments were purified by agarose gel extraction (Qiagen, Germany). PCR was performed in two rounds consisting of, respectively, 15 and 25 amplification cycles (94° C., 30 s; 55° C., 30 s; 72° C., 180 s). The purified fragments were used as the template for the second round of PCR using the two vector-specific primers. The final PCR product was purified and cloned into the expression vector pET17b (Novagen, Wis.) as described previously. Wu, H., Lima, W. F., and Crooke, S. T., 1998, Antisense Nucleic Acid Drug Dev. 8, 53-61. The incorporation of the desired mutations was confirmed by DNA sequencing. Point mutations and their positions are shown in FIG. 3A. The positional designations (shown in parentheses) refer to amino acid positions of E. coli RNase H1. Deletion mutants are shown in FIG. 3B.

### Example 13

### Protein Expression and Purification

The plasmid containing nucleic acid encoding mutant RNase H1 was transfected into E. coli BL21(DE3) (Novagen, Wis.). The bacteria were grown in M9ZB medium at 32° C. and harvested at OD₆₀₀ of 0.8. The cells were induced with 0.5 mM IPTG at 32° C. for 2 h. The cells are lysed in 8M urea solution and the recombinant protein was partially purified with Ni-NTA agarose (Qiagen, Germany).

The human RNase H1 was purified by C4 reverse phase chromatography (Beckman, System Gold, Fullerton, Calif.) using a 0% to 80% gradient of acetonitrile in 0.1% trifluoroacetic acid/distilled water (% v/v) over 40 min. Katayanagi et al., 1993, Proteins: Struct., Funct., Genet., 17, 337-346. The recombinant protein was collected, lyophilized and analyzed by 12% SDS-PAGE. The purified protein and control samples were re-suspended in 6 M urea solution containing 20 mM Tris-HCl, pH 7.4, 400 mM NaCl, 20% glycerol, 0.2 mM Phenylmethylsulfonyl fluoride (PMSF), 5 mM dithiothreitol (DTT), 10 μg/ml each aprotinin and leupeptin (Sigma, Mo). The protein was refolded by dialysis with decreasing urea concentration from 6 M to 0.5 M and DTT concentration from 5 mM to 0.5 mM. Cerritelli S. M. and Crouch, R. J., 1995, RNA, 1, 246-259. The refolded RNase H protein was concentrated 10-fold using a Centricon apparatus (Amicon, MA).

### Example 14

### Analysis of Purified Wild Type and Mutant RNase H1

Analysis of wild type and mutant human RNase H1 enzymes was carried out by SDS-polyacrylamide gel electrophoresis. As expected, mutant proteins containing amino acid substitutions, (e.g., D145N, E186Q, D210N, K226,227A and K226,227,231,236A) exhibited molecular weights similar to the 32 kDa wild-type. The RNase H1[ΔI] mutant in which the dsRNA-binding domain was deleted resulted in a 213 amino acid protein with an approximate molecular weight of 23 kDa. The deletion of the 62 amino acid center portion of human RNase H1 (RNase H1[ΔII]) resulted in a 224 amino acid protein with an approximate molecular weight of 25 kDa. Finally, the deletion of both the dsRNA-binding domain and the central region of the enzyme (RNase H1[ΔI-II]) resulted in a 151 amino acid protein containing the conserved E. coli RNase H1 sequence and with an approximate molecular weight of 17 kDa.

### Example 15

### Determination of Initial Cleavage Rates of DNA/RNA Substrate by RNase H1

The oligoribonucleotides were synthesized on a PE-ABI 380B synthesizer using 5′-O-silyl-2′-O-bis(2-acetoxyethoxy)methyl ribonucleoside phosphoramidites and procedures described elsewhere. Scaringe et al., 1998, J. Am. Chem. Soc. 120, 11820-11821/The oligoribonucleotides were purified by reverse-phase HPLC. The DNA oligonucleotides were synthesized on a PE-ABI 380B automated DNA synthesizer and standard phosphoramidite chemistry. The DNA oligonucleotides were purified by precipitation 2 times out of 0.5 M NaCl with 2.5 volumes of ethyl alcohol.

The RNA substrate is 5′-end-labeled with ³²P using 20 u of T4 polynucleotide kinase (Promega, Wis.), 120 pmol (7000 Ci/mmol) [γ-³²P]ATP (ICN, CA), 40 pmol RNA, 70 mM tris, pH 7.6, 10 mM MgCl₂ and 50 mM DTT. The kinase reaction is incubated at 37° C. for 30 min. The labeled oligoribonucleotide was purified by electrophoresis on a 12% denaturing polyacrylamide gel. The specific activity of the labeled oligonucleotide is approximately 3000 to 8000 cpm/fmol.

The heteroduplex substrate was prepared in 100 μL containing 50 nM unlabeled oligoribonucleotide, 10⁵ cpm of ³²P labeled oligoribonucleotide, 100 nM complementary oligodeoxynucleotide and hybridization buffer [20 mM tris, pH 7.5, 20 mM KCl]. Reactions were heated at 90° C. for 5 min, cooled to 37° C. and 60 u of Prime RNase Inhibitor (5 Prime→3 Prime, CO) and MgCl₂ at a final concentration of 1 mM were added. Hybridization reactions were incubated 2-16 h at 37° C. and β-mercaptoethanol (BME) was added at final concentration of 20 mM.

The heteroduplex substrate was digested with 0.5 ng human RNase H1 at 37° C. A 10 μl aliquot of the cleavage reaction was removed at time points ranging from 2-120 min and quenched by adding 5 μL of stop solution (8 M urea and 120 mM EDTA). The aliquots were heated at 90° C. for two min, resolved in a 12% denaturing polyacrylamide gel and the substrate and product bands were quantitated on a Molecular Dynamics PhosphorImager. The concentration of the converted product was plotted as a function of time. The initial cleavage rate was obtained from the slope (mole RNA cleaved/min) of the best-fit line for the linear portion of the plot, which comprises, in general <10% of the total reaction and data from at least five time points.

The initial cleavage rates (V₀) were determined for the human RNase H1 enzyme and the mutant proteins using a 17 nucleotide long RNA/DNA heteroduplex, (Table 5).

### Table 5

### Initial Cleavage Rates for Wild-Type and Mutant Human RNase H1 Proteins

Initial cleavage rates were determined as described in above. The initial cleavage rates are an average of n≧3 measurements. *Detection limit=cleavage rates resulting in <1% of the heteroduplex substrate over 60 min.

| Human RNase H1 Protein | V₀ (pM min⁻¹)* |
|---|---|
| Wild-type RNase H1 | 658 ± 130 |
| RNase H1[D145N] | below detection limit |
| RNase H1[E186Q] | below detection limit |
| RNase H1[D210N] | below detection limit |
| RNase H1[K226, 227A] | 8.1 ± 0.2 |
| RNase H1[K226, 227, 231, 236A] | below detection limit |
| RNase H1 [ΔI] | 488 ± 38 |
| RNase H1[ΔII] | 11 ± 2 |
| RNase H1[ΔI-II] | 610 ± 20 |

Substitution of any one of the three amino acids comprising the proposed catalytic site of human RNase H1, (e.g., [D145N], [E186Q], and [D210N]) ablated the cleavage activity of the enzyme. The RNase H1 [K226,227A] mutant exhibited an initial cleavage rate almost two orders of magnitude slower than the rate observed for the wild-type enzyme. The alanine substitution of two remaining lysine residues within the basic substrate binding domain (RNase H1 [K226,227,231,236A]) resulted in cleavage activity below the detection limit of the assay.

The complete ablation of cleavage activity observed for the RNase H1[D145N], [E186Q] and [D210N] mutants indicates that all three of the conserved residues in human RNase H1 are required for catalytic activity (Table 5). The fact that the RNase H1[D145N] mutant competitively inhibited the activity of human RNase H1 suggests that the loss in cleavage activity observed for this dominant negative mutant protein was not due to a loss in the binding affinity for the heteroduplex substrate. Taken together these data suggest that, consistent with the E. coli enzyme, the three conserved residues likely form the catalytic site of the enzyme and are not involved in the substrate-binding interaction.

The alanine substitution of all four lysine residues within the putative substrate-binding domain of human RNase H1 (RNase H1[K226,227,231,236A]) resulted in the complete loss of RNase H activity. Furthermore, the RNase H1[K226,227,231,236A] mutant was shown to competitively inhibit the cleavage activity of wild-type human enzyme, suggesting that the observed loss of RNase H activity for the mutant protein was not due to a loss in the overall binding affinity of the mutant protein for the substrate.

The initial cleavage rate for the RNase H1[ΔI] mutant in which the dsRNA-binding domain was deleted was 30% slower than the initial cleavage rate observed for the wild-type enzyme (Table 5). Region II comprises the amino acid sequence between the dsRNA-binding domain (region I) and the conserved E. coli RNase H1 domain (region III). Deletion of this region (RNase H1[ΔII]) resulted in a significant loss in the cleavage activity when compared to the wild-type enzyme. The RNase H1[ΔII] mutant was also shown to competitively inhibit the cleavage activity of human RNase H1 suggesting that the loss in RNase H activity did not appear to be due to a reduction in the binding affinity of the RNase H1[ΔII] mutant for the heteroduplex substrate. The initial cleavage rate observed for the wild-type enzyme was approximately 60-fold faster than the rate observed for the RNase H1[ΔII] mutant, a dominant negative. Conversely, the initial cleavage rate for the mutant protein in which both regions I and II were deleted (RNase H1[ΔI-II]) was comparable to the initial cleavage rate observed for the wild-type enzyme.

Region III, as represented by the H1[ΔI-II] mutant, contains the conserved E. coli RNase H1 domain. The cleavage rate observed for the H1[ΔI-II] mutant was comparable to the rate observed for wild-type human enzyme (Table 5), but approximately two-orders of magnitude slower than the cleavage rate observed for E. coli RNase H1 (Lima and Crooke (1997), Biochemistry 36, 390-398. The robust activity of the RNase H1[ΔI-II] mutant indicates that region III is capable of folding into an active structure independent of regions I and II and further suggests that region III constitutes an autonomous sub-domain of the human enzyme.

### Example 16

### Competitive Inhibition of RNase H Cleavage

Experiments were performed to determine whether the inactive mutants of human RNase H1 competitively inhibit the cleavage activity of the wild-type enzyme, i.e., whether they are dominant negative mutants. These experiments were performed with the enzyme concentration in excess of the substrate concentration and with the concentration of the mutant protein in excess of the wild-type enzyme concentration. Competition experiments were performed as described for the determination of initial rates with the exception that 20 nM oligodeoxynucleatide, 10 nM oligoribonucleotide and 2.5 ng of the mutant RNase H1 protein. Reactions were digested with 250 pg of wild-type Human RNase H. The reactions were quenched, analyzed and quantitated as described for the determination of initial rates.

All three of the mutant proteins tested were observed to competitively inhibit the cleavage activity of human RNase H1. For example, the initial cleavage rate of human RNase H1 alone was determined to be 6-fold faster than the initial cleavage rate for human RNase H1 in the presence of the RNase H1[D145N] mutant. The initial cleavage rate of human RNase H1 in the presence of the region II deletion mutant (RNase H1[ΔII]) was approximately 50% slower than the rate observed for human RNase H1 alone. Finally, the initial cleavage rate for human RNase H1 in the presence of the RNase H1[K226,227,231,236A] mutant was approximately 60% slower than the rate observed for human RNase H1 alone.

### Example 17

### Binding Affinities of RNase H1 Enzyme for Substrate

Binding affinities were determined by inhibition analysis. Lima and Crooke, 1997, Biochemistry 36, 390-398. The RNA-DNA heteroduplex was prepared as described above except in a final volume of 60 μL and with the concentration of the heteroduplex ranging from 10 nM to 500 nM. The non-cleavable heteroduplex substrate was prepared in 60 μL of hybridization buffer containing equimolar concentrations of oligodeoxynucleotide and complementary 2′-fluoro modified oligonucleotide in excess of the RNA-DNA hybrid. The DNA-2′-fluoro duplex was added to the RNA-DNA duplex and the combined reaction was digested with human RNase H1 as described for the determination of initial rates. The reactions were quenched, analyzed and quantitated as described for the determination of initial rates.

The binding affinities of human RNase H1 and the RNase H1[ΔI-II] mutant were determined indirectly using a competition assay as previously described. Lima and Crooke, 1997, Biochemistry 36, 390-398. Briefly, the cleavage rate of the RNA/DNA heteroduplex was determined at a variety of substrate concentrations in both the presence and absence competing noncleavable DNA/2′F heteroduplex. The dissociation constant (K_{d}) of human RNase H1 for the DNA/2′F heteroduplex was 75 nM. The RNase H1[ΔI-II] mutant exhibited a K_{d} of 126 nM for the DNA/2′F heteroduplex (Table 6).

TABLE 6
Binding Constants of RNase H1 proteins.
K_{d} measurements were determined as described in
Materials and Methods. The K_{d} value for E. coli RNase H1 was
derived from previously reported data. Lima and Crooke (1997)
Biochemistry 36, 390-398. The dissociation constants for
human RNase H1 proteins are derived from ≧2 slopes of
Lineweaver-Burk analysis.

| RNase H1 Protein | K_{d} (nM) |
|---|---|
| Human RNase H1[ΔI-II] | 75 ± 8 |
| Human RNase H1 [ΔI-II] | 126 ± 22 |
| E. coli RNase H1 | 1600 |

The cleavage activity of the RNase H1[ΔI] and [ΔI-II] mutants suggests that the enzyme does not require the dsRNA-binding domain in order to bind to the heteroduplex substrate. In fact, the binding affinity of the wild-type human enzyme for the heteroduplex substrate was <2-fold tighter than the RNase H1[ΔI-II] mutant without the dsRNA-binding domain (Table 6).

### Example 18

### Position of RNA/DNA Substrate Cleavage

As previously observed, wild type human RNase H1 exhibited a strong positional preference for cleavage of the RNA/DNA substrate, i.e., 8 to 12 nucleotides from the 5′-RNA/3′-DNA terminus of the duplex. A similar cleavage pattern was observed for both the RNase H1[K226,227A] substitution mutant and the RNase H1[ΔII] deletion mutant. The RNase H1[ΔI] and H1[ΔI-II] deletion mutants exhibited broader cleavage patterns on the heteroduplex substrate, with cleavage sites ranging from 7 to 13 nucleotides from the 5′-terminus of the RNA.

The cleavage pattern for the mutants in which Region I (the dsRNA-binding region) was deleted (RNase H1[ΔI] and [ΔI-II]) differed from the pattern observed for the wild-type human enzyme. In fact the cleavage pattern for the RNase H1[ΔI] and [ΔI-II] mutants resembled the cleavage pattern of the E. coli RNase H1 enzyme which does not contain a dsRNA-binding domain. Taken together these data suggest that the dsRNA-binding domain is responsible for the observed strong positional preference for cleavage exhibited by human RNase H1, (Wu et al., 1999, J. Biol. Chem. 274, 28270-28278) and further suggest that this region contributes to the overall binding affinity of the enzyme for the substrate and the regulation of the sites of cleavage. Finally, the broad cleavage pattern observed for the RNase H1[ΔI-II] mutant further suggests that the strong positional preference for cleavage displayed by human RNase H1 is not responsible for slower cleavage rate of the human enzyme compared to E. coli RNase H1. The cleavage rate observed for human RNase H1 was approximately two orders of magnitude slower than the rate observed for the E. coli enzyme. Lima and Crooke, 1997, Biochemistry 36, 390-398. The strong positional preference for cleavage displayed by human RNase H1 in effect limits the number of productive binding interactions for a given substrate.

### Example 19

### Antibodies

Two human RNase H1 peptides, H-CRAQVDRFPAARFKKFATED-OH (amino acids 46-65; SEQ ID NO: 49) corresponding to N-terminal region and H-CKTSAGKEVINKEDFVALER-OH (amino acids 231-249; SEQ ID NO: 50), corresponding to the C-terminus of the full RNase H1 protein (SEQ ID NO: 1) were conjugated to diphtheria toxin with maleimidocaproyl-N-hydroxysuccinamide and used to raise polyclonal antibodies in rabbits. The anti-N-terminus and anti-C-terminus antibodies (IgGs) were affinity purified using the antigenic peptide coupled to thiopropyl-Sepharose 6B (Harlow, E. and Lane, D., 1988, Antibodies. A Laboratory Manual, Cold Spring Harbor, N.Y.). Polyclonal antibodies to the His-tagged human RNase H1 (amino acids 73-286) and full length human RNase H2 were also raised. Both proteins used to raise polyclonal antibodies were more than 95% pure. Polyclonal antibodies were further purified with each protein antigen using Aminolink immobilization kits (Pierce, Rockford Ill.) 200 μg purified H1 and H2 antibodies were then directly immobilized on agarose gel by using SEIZE primary immunoprecipitation kit (Pierce, Rockford Ill.) to create a permanent affinity support for immunoprecipitation without the need of protein A or protein G beads.

### Example 20

### Western Blot Analysis

Whole cell lysates and non-nuclear or nuclear fractions from cells or mouse liver were prepared (Dignam et al., 1983, Nucl. Acids Res. 11, 1475-1489). Protein concentrations in lysates were measured by the Bradford method (Bio-Rad Lab, Hercules Calif.). Samples were boiled in SDS-sample buffer and separated by SDS-PAGE using 4-20% Tris-glycine gels (Invitrogen, Carlsbad Calif.) under reducing conditions. Pre-stained molecular weight markers were used to determine the protein sizes. The proteins were electrophoretically transferred to PVDF membrane and processed for immunoblotting using the appropriate affinity purified RNase H antibody at 0.5-1 μg/ml. The immunoreactive bands were visualized using the enhanced chemiluminescence method (Amersham, Arlington Heights Ill.) and analyzed using Phosphorimager Storm 860 (Molecular Dynamics, Sunnyvale Calif.).

### Example 21

### Immunoprecipitation and Enzyme Activity Assay

To analyze human RNase H1 and H2 activities, cells were lysed in RIPA buffer (150 mM NaCl, 10 mM Tris, pH 7.2, 0.1% SDS, 1.0% Triton X-100, 1% deoxycholate, 5 mM EDTA) and protein concentrations were measured using the Bradford method (Bio-Rad, Hercules Calif.). Immunoprecipitation was performed with purified rabbit anti-human RNase H1 or H2 antibody (10 or 25 μg antibody/mg cell lysate). The immunoprecipitated samples were analyzed either by Western blot, trichloroacetic acid precipitation assay or denaturing polyacrylamide gel electrophoresis. The renaturation gel assay for in situ detection of RNase H activity was carried out in the presence of Mn²⁺ or Mg²⁺ as described by Frank et al., 1993, Biochim. Biophys. Acta, 196, 1552-1557. Autoradiograms were analyzed using PhosphorImager Storm 860 (Molecular Dynamics, Sunnyvale Calif.).

### Example 22

### Indirect Immunofluorescence Staining

Both untreated HeLa cells and HeLa cells transfected with adenovirus vectors were cultured in chamber slides for immunostaining. Cells were washed once with D-PBS (pH 7.0) and then fixed in 10% neutral-buffered formalin for ten minutes followed by washing three times with D-PBS. Fixed cells were then blocked for 30 minutes with 20% fetal bovine serum plus 0.5% Tween-20. Cells were first stained with purified anti-RNase H1 antibody, anti-RNase H2 antibody or normal rabbit IgG (10 μg/ml) for 1 hour at 37° with the FITC goat anti-rabbit IgG (Jackson ImmunoResearch Laboratory, Inc., West Grove Pa.). The cells were washed with D-PBS three times and mounted in mounting medium (Vector, Burlingame Calif.) for examination under a fluorescence microscope.

### Example 23

### Overexpression of Human RNase H1 and H2

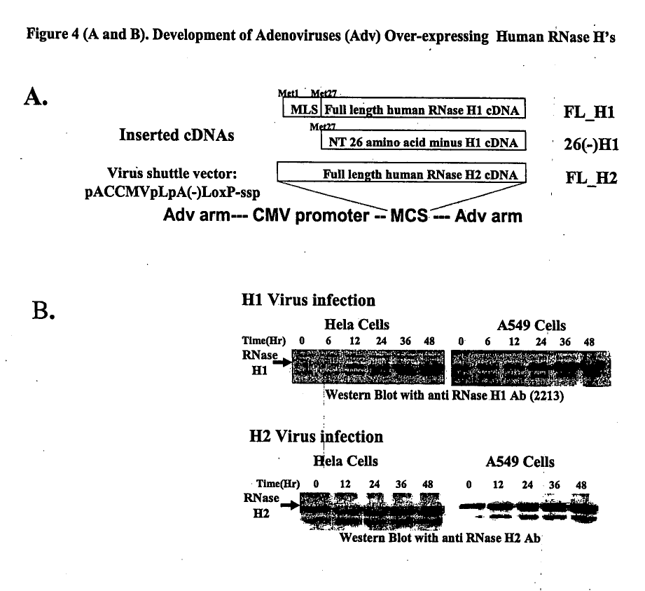

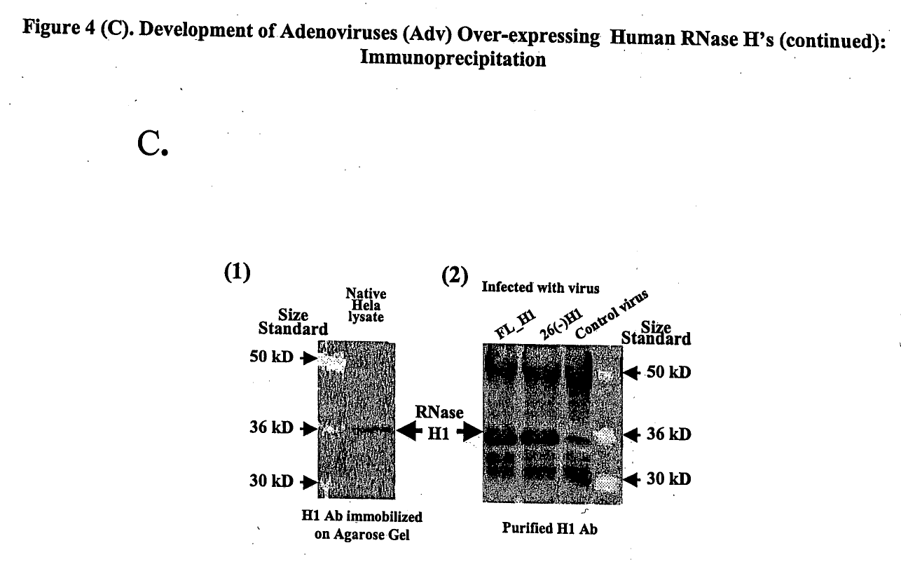

For overexpression of human RNase H1 and H2, three strains of adenoviruses containing RNase H inserts were developed (FIG. 4A). The first contained the full length cDNA for human RNase H1, the second contained the full length cDNA for human RNase H2, and the third contained the full length cDNA for the 26aa minus RNase H1[26−], a mutant which was constructed as in above examples and lacks amino acids 1-26 which are believed to be the suggested mitochondrial localization signal(MLS), was amplified by PCR and inserted in sense orientation into an adenovirus shuttle vector, pACCMVpLpA(−)LoxP-ssp (The Vector Core Laboratory of University of Michigan Medical Center, Ann Arbor Mich.) into EcoRI and XhoI sites in the multiple cloning site (MCS) downstream from the CMV promotor. Each insert fragment was confirmed by DNA sequencing, then the adenoviruses were generated by the Vector Core Lab of the University of Michigan. The viruses and the control virus (LoxP) were prepared by either cell lysate (titration to 3-7×10⁹ pfu/ml) or CsCl purified cell lysates (titration 1.38 to 1.61×10¹¹ pfu/ml.

Human cell lines HeLa, A549 and HepG2 cells (ATCC, Manassas Va.) were cultured in DMEM supplemented with 10% fetal bovine serum in 6 well or 96 well culture plates or 10 cm or 15 cm culture dishes. Human cell lines MCF7 and T24 cells (ATCC) were cultured in McCoy's medium with 10% fetal bovine serum. Mouse AML12 and HeLa cells were also grown in DMEM with 0.005 mg/ml insulin, 0.005 mg/ml transferrin, 5 ng/ml selenium, 40 ng/ml dexamethasone and 10% fetal bovine serum. Medium and supplements were purchased from Invitrogen (Carlsbad Calif.). For adenovirus infection, virus (10-400 pfu/cell) was added directly into cell culture.

Western blot analysis was performed on protein lysates from HeLa or A549 cells infected with full length H1 or H2 virus (200 pfu/cell). The cells were harvested at 0, 6, 12, 24, 36, 48 and 72 hours following virus infection. The protein concentrations of the cell lysates were measured. The lysates were subjected to 4-20% gradient SDS-PAGE (20 μg/lane) and western blot analysis was performed with antibodies to human RNase H1 (antibody 2213, against C-terminal peptide) and RNase H2. The RNase H1 virus may use the first (met1) or the second (met27) methionine to start protein translation.

FIG. 4B shows that both full length RNase H1 and RNase H2 were overexpressed in HeLa and A549 cells. Peak expression was observed 36-48 hours after infection. In addition, all three enzymes could be overexpressed in T24, MCF7, HepG2 and H293 cells.

To compare the full length RNase H1 and the RNase H1[26−] proteins, the purified human RNase H1 antibody was used to immunoprecipitate the enzyme from untreated HeLa cell and adenovirus infected HeLa cell lysates. FIG. 4C shows a western blot of the immunoprecipitated lysate from untreated HeLa cells and demonstrates that both full length human RNase H1 and H1[26−] virally produced enzymes were overexpressed and comigrated with the enzyme from uninfected cells. (Panel 1) Immunoprecipitation was performed using untreated HeLa cell lysate with purified human RNase H1 antibody which was covalently immobilized to agarose beads. The eluted samples were subjected to Western blot analysis with RNase H1 antibody. (Panel 2) HeLa cells were infected with virus containing a plasmid containing the full length or N-terminal 26-amino-acid-minus RNase H1, or the control virus LoxP. Cell lysates were prepared after 24 hours of infection and subjected to immunoprecipitation with RNase H1 antibody (10 μg antibody/mg protein lysate). That the human RNase H1[26−] enzyme comigrated with the full length may be explained either by the use of the alternative start codon at amino acid 27 or by rapid processing of the terminal 26 amino acid peptide.

Additional mutants of RNase H1 were constructed, in which point mutations in the active site of the enzyme are created and the rest of the gene is left intact. The goal is an enzyme that is inactive catalytically, yet binds to the substrate to compete out the natural enzyme and reduce RNase H activity in the cell. These mutants are called dominant negative mutants.

A mutant (#48E->Q), in which amino acid 48 of the 286-amino acid human RNase H1 was changed from glutamic acid to glutamine, was prepared as described in previous examples. Another mutant (#70D->N), in which amino acid 70 was changed from aspartic acid to asparagine, was prepared similarly. See FIG. 3A for location of mutations; amino acid position refers to corresponding position on E. coli RNase H1. Mutants were also prepared which had one of the point mutations as well as the “minus26aa” deletion of amino acids 1-26 described in previous examples. These mutants were designated “26aa minus 48 E->Q” or “−26aa 48 E->Q” and “26aa minus 70 D->N” or “−26aa 70 D->N”, respectively.

cDNA for each of these mutated forms of human RNase H1 is inserted into an adenovirus shuttle vector and used to transfect HeLa cells as described above. The transfected cells thus overexpressed one of the mutant forms of human RNase H1.

A western blot of cells transfected with the full length 48 E->Q mutant and the −26 aa 48 E->Q mutant showed that both the full length (FL) 48 E->Q mutant and the shorter −26aa 48 E->Q mutant were overexpressed and react with both C-terminal and N-terminal reactive antibodies.

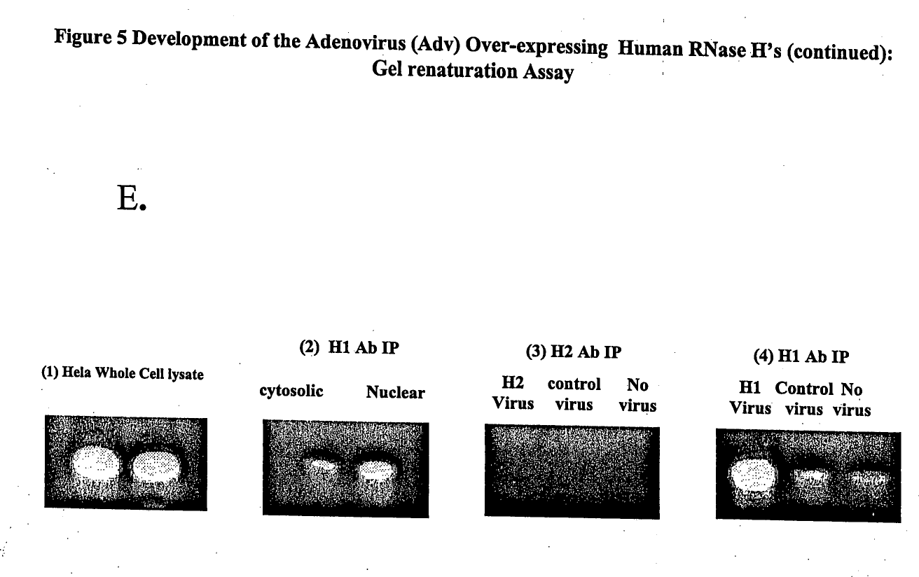

To determine if the overexpressed proteins were active, we employed a gel renaturation assay (Frank et al., 1993, Biochim. Biophys. Acta, 196, 1552-1557). As previously reported, human RNase H1 can be renatured and was active in the renaturation assay. The immunoprecipitated material (RNase H1) was separated on a renaturing polyacrylamide gel which separates the proteins by size as they renature in the gel. The gel matrix itself is impregnated with DNA-RNA duplexes (a substrate for RNase H1), and cleavage of the substrate is detectable in the gel. Thus RNase H1 cleavage activity can be correlated with protein size. Results are shown in FIG. 5. (Panel 1) 5 μg of uninfected HeLa cell lysate; (Panel 2) samples from immunoprecipitation with RNase H1 antibody from HeLa cell nuclear and cytosolic extracts; (Panel 3) samples from immunoprecipitation with RNase H2 antibody from the lysates of HeLa cells infected without or with adenovirus vector for RNase H2, or with control virus; (Panel 4) samples from immunoprecipitation with RNase H1 antibody from the lysates of HeLa cells infected without or with virus containing RNase H1 or control virus.

Human RNase H1 activity was present in both the cytosolic and nuclear fractions of uninfected HeLa cells. To confirm that the activity was indeed human RNase H1, the enzyme was immunoprecipitated from HeLa cells, and then subjected to the gel renaturation assay. Overexpression of the full length human RNase H1 or RNase H1[−26] resulted in increased activity in the gel renaturation assay. In contrast to RNase H1, neither endogenous nor overexpressed human RNase H2 was active in the gel renaturation assay.

In uninfected HeLa cells, in situ immunofluorescence experiments showed that both human RNase H1 and H2 were located primarily in the nucleus. However, RNase H2 could readily be detected in the cytosol and small amounts of RNase H1 were observed in the cytosol as well. Overexpressed human RNase H2 localized to the nucleus but was also present in the cytosol. In contrast, human RNase H1[26−] was localized strictly in the nuclei of the HeLa cells.

### Example 24

### Immunoprecipitation Assay for Human RNase H2

A problem in the study of mammalian RNase H2 until now has been the fact that cloned, expressed and purified human RNase H2 has been only marginally active, or inactive, in the gel renaturation or solution-based assays. While not wishing to be bound by theory, this may be due to the lack of associated proteins necessary for enzyme activity or because the enzyme's conformation is incorrectly reformed when expressed or purified. To overcome this limitation, we immunoprecipitated RNase H2 from HeLa cells using purified antibodies to human RNase H2, then analyzed the activity either by trichloroacetic acid (TCA) precipitation assay or gel electrophoresis. Extraction of proteins from the immunoprecipitation beads followed by polyacrylamide gel electrophoresis demonstrated that a number of proteins immunoprecipitated with human RNase H2. To support comparisons between the human RNase H1 and H2, we developed a similar approach for human RNase H1.

HeLa cells were infected with human RNase H1, H2 or control virus (200 pfu/cell) in 10 cm plates in quadruplicate for 24 hours before harvest. Cell lysates were prepared and protein concentrations were measured. 0.7 mg protein lysate was used for immunoprecipitation with RNase H1 antibody (15 μg RNase H1 antibody/mg protein lysate) or 0.35 mg per tube for RNase H2 antibody (30 μg antibody per mg protein). One set of the immunoprecipitated samples was eluted in 2×SDS loading buffer (Invitrogen, Carlsbad Calif.) and subjected to SDS-PAGE and western blot with RNase H1 or H2 antibody. The other three sets of immunoprecipitated samples were used in the RNase H activity assay against 50 nM of a 17mer Ras RNA/DNA duplex substrate (sense strand is 5′-end labeled oligoribonucleotide ISIS 3058, GGGCGCCGUCGGUGUGG; SEQ ID NO: 22; antisense strand is ISIS 4701, CCACACCGACGGCGCCC; SEQ ID NO: 51). The digested duplexes were subjected to TCA precipitation and the radioactivity in supernatants was determined for the digested RNA fragments by scintillation counting. The experiments were performed in triplicate and repeated three times. The error bars show standard error of the mean.

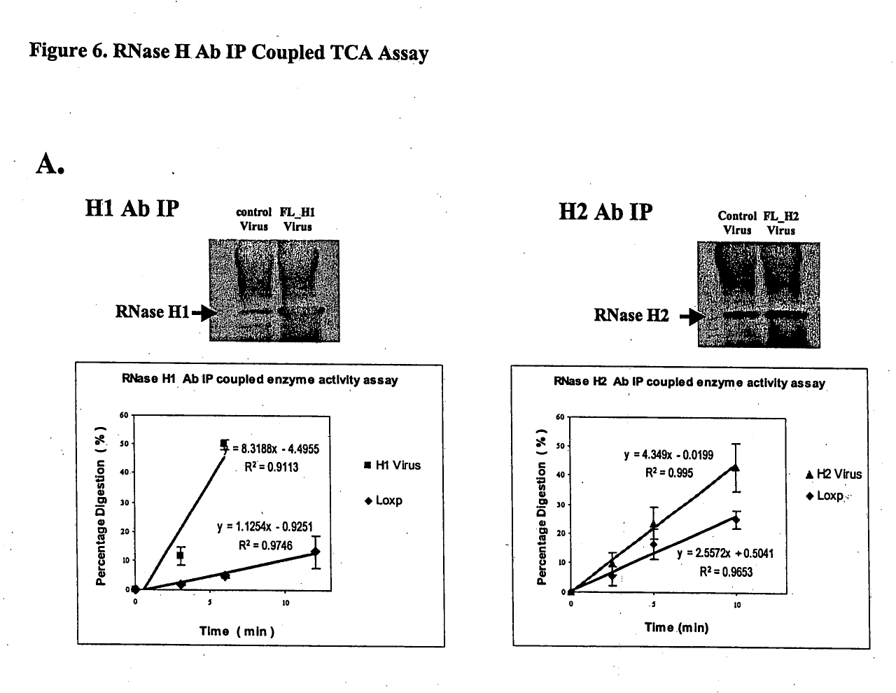

FIG. 6 demonstrates that both human RNase H1 and H2 are active in the TCA assay after immunoprecipitation. Further, when the enzymes were overexpressed, the activity extracted from the HeLa cells was greater, confirming that for both RNase H1 and RNase H2 the overexpressed enzymes were active.

To confirm these observations and to determine if the enzymes display different site preferences, the cleavage patterns of human RNase H1 and H2 immunoprecipitated from uninfected HeLa cells were compared, using two different RNA-DNA duplex substrates. A 17mer Ras RNA/DNA duplex substrate described above and a 20mer human Bcl-x RNA/DNA duplex substrate [sense strand (RNA) is ISIS 183349; ACUGUGCGUGGAAAGCGUAG; SEQ ID NO: 52; antisense strand (DNA) is ISIS 17619; CTACGCTTTCCACGCACAGT; SEQ ID NO: 53] were prepared and subjected to digestion by the RNase H1 or H2 antibody-immunoprecipitated samples from untreated HeLa cells for different time periods at 37° C. The digested duplexes were subjected to denaturing polyacrylamide gel electrophoresis.

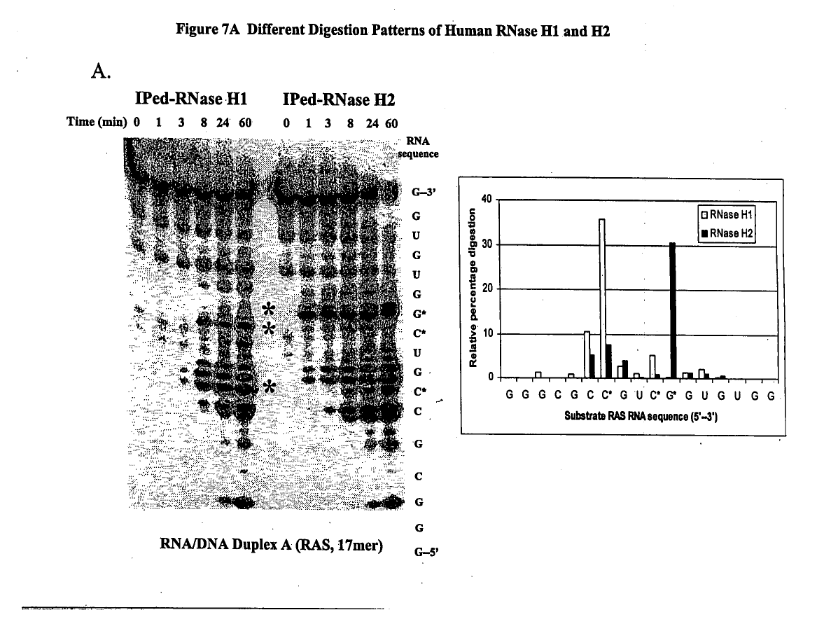

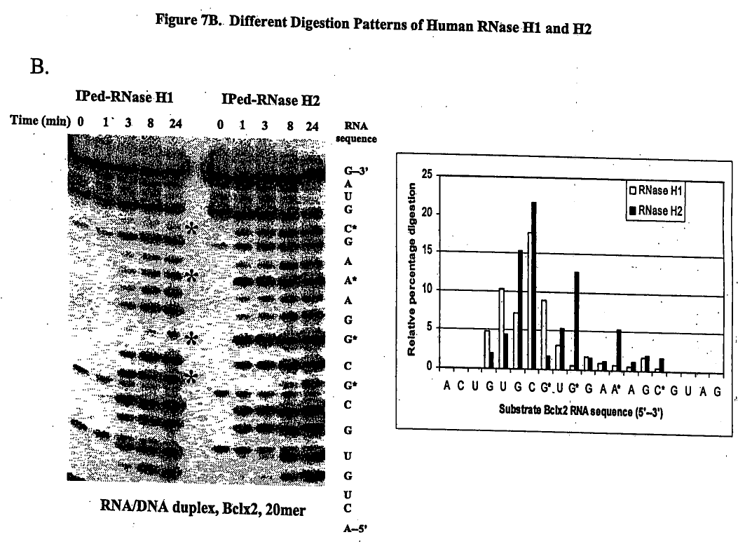

FIG. 7 demonstrates that the enzymes display different cleavage patterns in both substrates. Further, the cleavage pattern observed for immunoprecipitated human RNase H1 was identical to that observed previously with purified RNase H1. Panel 1 (left) shows cleavage of the 17mer Ras duplex. Panel B1 (right) shows the relative extents of digestions at each position of the substrate calculated with the PhosphorImager and compared for RNase H1 and H2. Panel 2 shows cleavage of the 20mer Bcl-x duplex.

### Example 25

### Northern Blot Analysis

Total RNA was isolated from cultured human cells using RNAeasy kits (Qiagen, Valencia Calif.). 5-10 μg of total RNA were separated on a 1.2% agarose/formaldehyde gel and transferred to Hybond-N+ (Amersham, Arlington Heights Ill.), and fixed to the membrane using a UV crosslinker (Stratagene, La Jolla Calif.). Hybridization was performed by using ³²P-labeled human RNase H1, G3PDH or c-Raf DNA probes in Quik-Hyb buffer (Stratagene, La Jolla Calif.) at 68° for 2 hours. After hybridization, membranes were washed in a final stringency of 0.1×SSC/0.1% SDS at 60° C. for 30 minutes. Membranes were analyzed using PhosphorImager Storm 860 (Molecular Dynamics, Sunnyvale Calif.).

### Example 26

### RT-PCR Analysis of Cellular Target RNA Levels

Total RNA was isolated from cultured human cells using an RNAeasy 96 kit (Qiagen, Valencia Calif.) and a BioRobot 3000 (Qiagen) according to the manufacturer's protocol. The RNA concentration was measured with Ribogreen RNA quantitation reagent (Molecular Probes, Eugene Oreg.). Gene expression was analyzed using quantitative RT/PCR as described in Winer et al., 1999, Anal. Biochem., 270, 41-49. Total RNA was analyzed in a final volume of 50 μl containing 200 nM gene-specific PCR primers, 0.2 mM of each dNTP, 75 nM fluorescently labeled oligonucleotide probe, 1×RT/PCR buffer, 5 mM MgCl₂, 2 U Platinum Taq DNA Polymerase (Invitrogen, Carlsbad Calif.) and 8 U ribonuclease inhibitor. Reverse transcription was performed for 30 minutes at 48° C. followed by PCR: 40 thermal cycles of 30 sec at 94° C. and 1 minute at 60° C. using an ABI Prism 7700 Sequence Detector (Foster City Calif.). The following primer/probe sets were used (a published target sequence for each is given by public database accession number):

Human c-Raf kinase (accession number X03484):

| | |
|---|---|
| forward primer- | |
| AGCTTGGAAGACGATCAGCAA | (SEQ ID NO: 54) |
| reverse primer- | |
| AAACTGCTGAACTATTGTAGGAGAGATG | (SEQ ID NO: 55) |
| probe- | |
| AGATGCCGTGTTTGATGGCTCCAGCX | (SEQ ID NO: 56) |

Human PTEN phosphatase (accession number U92436):

| | |
|---|---|
| forward primer- | |
| AATGGCTAAGTGAAGATGACAATCAT | (SEQ ID NO: 57) |
| reverse primer- | |
| TGCACATATCATTACACCAGTTCGT | (SEQ ID NO: 58) |
| probe- | |
| TTGCAGCAATTCACTGTAAAGCTGGAAAGGX | (SEQ ID NO: 59) |

Human JNK2 protein kinase (accession number U35003.1)

| | |
|---|---|
| forward primer- | |
| CGCTGGCCTCAGACACAGA | (SEQ ID NO: 60) |
| reverse primer- | |
| CTAACCTATCATCGACAGCCTTCA | (SEQ ID NO: 61) |
| probe- | |
| AGCAGTCTTGATGCCTCGACGGGAX | (SEQ ID NO: 62) |

Human RNase H1 (accession number AF039652)

| | |
|---|---|
| forward primer- | |
| GGTTTCCTGCTGCCAGATTTAA | (SEQ ID NO: 63) |
| reverse primer- | |
| GGCTTGCAGATTTCCTGACAA | (SEQ ID NO: 64) |
| probe- | |
| TTTGCCACAGAGGATGAGGCCTGGX | (SEQ ID NO: 65) |

Human RNase H2 (accession number NM_{—}006397)

| | |
|---|---|
| forward primer- | |
| CCCGTTCTTCCCACCGATA | (SEQ ID NO: 66) |
| reverse primer- | |
| GCTGCTAGAGGCTGGTTGCT | (SEQ ID NO: 67) |
| probe- | |
| TTCCTGGAACGCGGCCTGGAX | (SEQ ID NO: 68) |

Mouse JNK1 protein kinase (accession number BU611812.1)

| | |
|---|---|
| forward primer- | |
| CAACGTCTGGTATGATCCTTCAGA | (SEQ ID NO: 69) |
| reverse primer- | |
| GTGCTCCCTCTCATCTAACTGCTT | (SEQ ID NO: 70) |
| probe- | |
| AAGCCCCACCACCAAAGATCCCGX | (SEQ ID NO: 71) |

where X indicates the presence of reporter dye (e.g., FAM or JOE, obtained from PE-Applied Biosystems, Foster City, Calif., Operon Technologies Inc., Alameda Calif. or Integrated DNA Technologies Inc., Coralville Iowa) on the 3′ end of the probe.

### Example 27

### Overexpression of Human RNase H1 Increases the Potency of Antisense Oligonucleotides In Vitro

To evaluate the effect of overexpression of RNase H on the potency of antisense oligonucleotides, HeLa and A549 cells were infected with either the control (LoxP) adenovirus or adenovirus containing either the human RNase H1 or H2 insert, after which the effects of several well characterized DNA-like antisense oligonucleotides on inhibition of levels of their respective intracellular target mRNA were determined. The antisense oligonucleotides used were ISIS 13650 (TCCCGCCTGTGACATGCATT; SEQ ID NO: 72), targeted to human c-Raf; ISIS 101759 (GCTCAGTGGACATGGATGAG; SEQ ID NO: 73), targeted to human JNK2, and ISIS 116847 (CTGCTAGCCTCTGGATTTGA; SEQ ID NO: 74), targeted to human PTEN. Each of these is a chimeric “gapped” oligonucleotide which has a 2′-O-methoxyethyl (2′-MOE) modification at each position shown in bold, and 2′-deoxy (unmodified) nucleotides in the remaining positions. All 2′-MOE cytosines were 5-methyl cytosines and in ISIS 116847 all 2′-deoxycytosines were also 5′methylcytosines.

HeLa cells were split into 6000 cells/well in 96 well plates, then infected with RNase H1, H2 or control (LoxP) adenovirus at 200 pfu/cell. Twelve hours later, the cells were transfected with the anti-c-Raf antisense oligonucleotide, ISIS 13650, at varying concentrations. The cells were harvested 24 hours later. c-Raf mRNA levels were measured with RT-PCR in which the reverse transcription and PCR amplification of c-Raf mRNA were performed in 96-well format with the primer set described above. The IC₅₀s were calculated and presented under the graphs. The bars represent standard error of the mean of 3-5 replicates of a representative experiment.

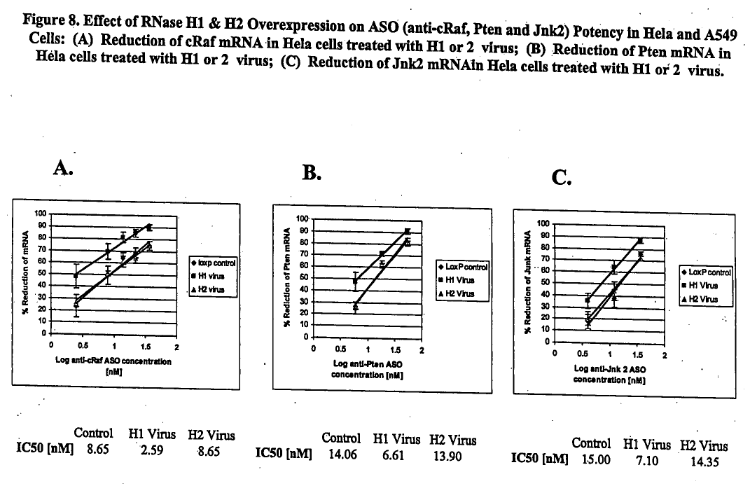

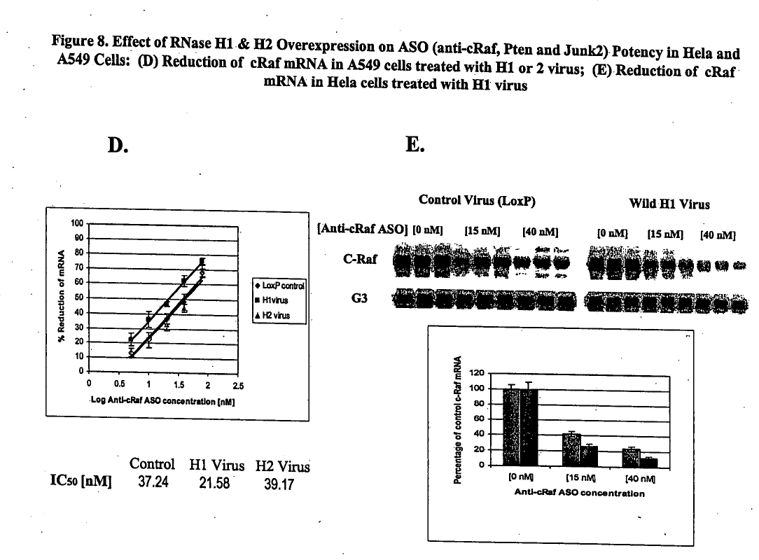

FIG. 8 shows that the potencies of antisense oligonucleotides designed to bind to human c-Raf, PTEN or JNK2 were significantly increased by overexpression of human RNase H1 in HeLa cells. Overexpression of RNase H2 had no effect on antisense potency. FIG. 8A shows results for the c-Raf antisense oligonucleotide. FIGS. 8B and 8C show similar experiments with the PTEN antisense oligonucleotide and the JNK2 antisense oligonucleotide, respectively. Similar effects were seen in A549 cells (FIG. 8D) and whether RT-PCR or Northern blot analysis was used to measure target RNA levels. FIG. 8E shows Northern blot analysis of the effects of RNase H1 on the potency of the c-Raf antisense oligonucleotide in HeLa cells. The cells were split into 1×10⁶ cells per 10 cm plate and incubated with control or RNase H1 expressing virus (200 pfu/cell) for 12 hours before the cells were transfected with anti-c-Raf antisense oligonucleotide (ISIS 13650) at varying concentrations, using Lipofectin. The cells were harvested 24 hours later and the total RNA was prepared with an RNAeasy kit (Qiagen, San Diego Calif.). 5 Ug RNA/lane was subjected to 1.2% agarose/formaldehyde electrophoresis and to Northern blot analysis with ³²P-labeled human c-Raf cDNA probe and G3PDH probe for normalization. The experiment was performed in triplicate and results were plotted with percentage normalized mRNA level vs. antisense oligonucleotide concentration. The bars represent standard error of the mean of the triplicates. The IC₅₀ value for each antisense oligonucleotide is shown under the graph.

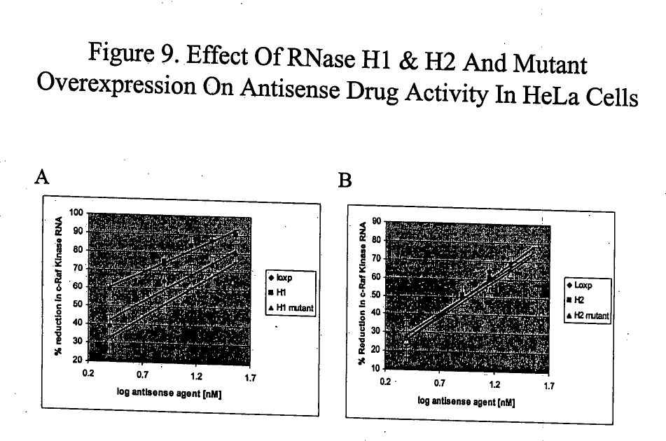

In contrast to the effect shown with the wild type RNase H1, overexpression of the dominant negative #48 E->Q RNase H1 mutant reduced the activity of antisense, using the c-Raf antisense target. This is shown in FIG. 9A. A dominant negative mutant of human RNase H2 was also prepared according to methods described above. As with wild type RNase H2, overexpression of a dominant negative mutant of RNase H3 had no effect on antisense activity in cells (FIG. 9B).

### Example 28

### Overexpression of Human RNase H1 Increases the Potency of Antisense Oligonucleotides In Vivo

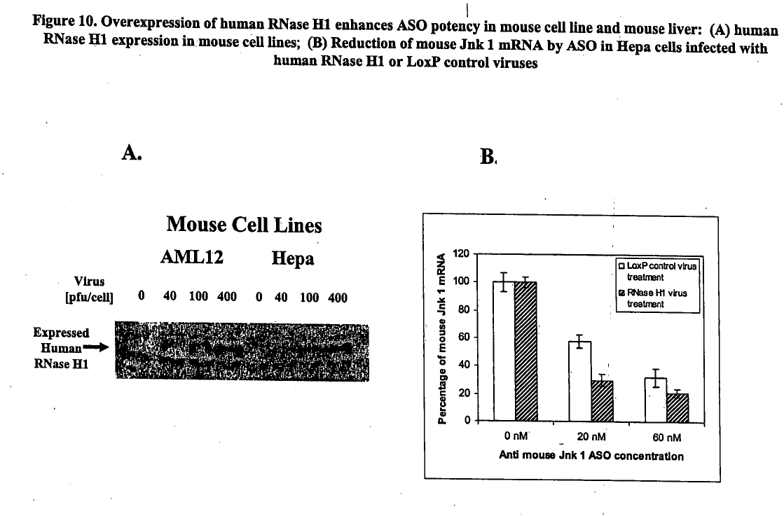

DNA-like antisense oligonucleotides are frequently used in in vivo experiments and are being evaluated in multiple clinical trials in humans. Experiments in mice were therefore conducted to examine the effects of overexpressing RNase H on potency of DNA-like antisense oligonucleotides in vivo. It was demonstrated (FIG. 10A) that both human RNase H1 and human RNase H2 could be overexpressed in mouse AML12 and Hepa cell lines. Adenoviral infection and western blot analyses were performed as described for human cell lines in previous examples. FIG. 10B shows that overexpression of human RNase H1 increased antisense oligonucleotide potency (using antisense oligonucleotide ISIS 104492, SEQ ID NO: 75, targeted to mouse JNK1) in mouse cells. Methods were as described in the previous examples except that virus titer was 400 pfu/cell. Overexpression of RNase H2 had no effect on antisense potency.

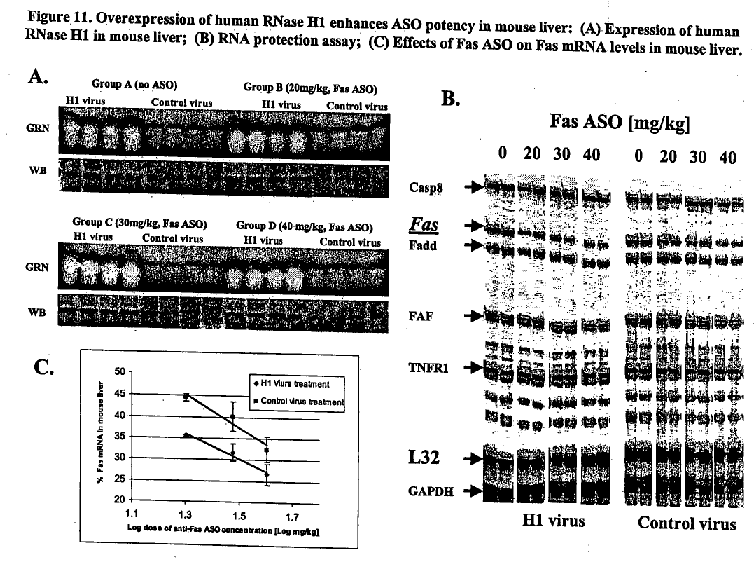

Groups of mice were then treated with the control and human RNase H1-containing adenovirus. Eight week old female Balb/c mice were purchased from Jackson Laboratory (Jackson Me.). Mice were treated with the adenovirus (6×10⁹ pfu in 200 μl PBS) by intravenous injection (i.v.), according to the indicated schedules. After 24 hours, animals were sacrificed and the livers harvested. Liver tissue lysate was prepared with SDS RIPA lysis buffer. 20 μg protein were used in the gel renaturation assay (GRN) in the presence of 10 mM Mg²⁺ and Western blot (WB) with antibody to human RNase H1. FIG. 11A shows that human RNase H1 was significantly overexpressed in the liver of the animals that were infected with the adenoviruses containing the insert. The human RNase H1 expressed in mouse liver was active in the gel renaturation assay. Moreover, the degree of overexpression was reasonably consistent. Each lane represents a sample from an individual animal (n=4 for each group).

To determine if overexpression of human RNase H1 in mouse liver increased the potency of DNA-like antisense oligonucleotides, the effects of a well characterized antisense oligonucleotide targeted to mouse Fas were evaluated. Mice were treated with antisense oligonucleotide targeted to mouse Fas (ISIS 22023; SEQ ID NO: 76) in saline (Gibco/BRL) or with saline alone in 200 μl by intraperitoneal injection (i.p.) four hours before treatment with the adenovirus (6×10⁹ pfu in 200 μl PBS) by intravenous injection (i.v.), according to the indicated schedules. Total RNA was extracted from mouse liver (same mice as in FIG. 11A) using RNAeasy kits (Qiagen, Valencia, Calif.). RNase protection assays (RPA) were performed according to the manufacturer's instructions (Pharmingen, San Diego Calif.) to quantitate Fas mRNA levels in the liver. RPA template mApo-3 and a custom template (Pharmingen) were used as probes. 20 μg total RNA was analyzed on 6% denaturing polyacrylamide gels. Individual transcripts were then quantitated on a PhosphorImager. Fas mRNA expression levels were normalized to L32 or GAPDH mRNA levels in each individual sample and presented as the percentage of saline (control) treated animals. FIG. 11B shows that the antisense oligonucleotide caused the selective reduction of Fas RNA in mouse liver and that overexpression of human RNase H1 increased the potency of the Fas antisense oligonucleotide. The figure shows two lanes for each group (n=4). Fas and other RNAs are labeled to the left of the figure.

A comparison of the dose response curves is shown in FIG. 11C. Effects of different doses of Fas antisense oligonucleotide on Fas mRNA levels were compared with saline control after normalization to L32 mRNA. The bars represent the standard error of the mean of four animals in each group. The experiment was repeated three times with equivalent results. The effects of overexpression of human RNase H1 were further confirmed by immunostaining of Fas protein with a Fas antibody. Liver sections from RNase H1 and control virus-treated mice were processed for immunohistochemical staining using a Fas-specific monoclonal antibody. Liver tissue was embedded in OCT (Baxter) followed by freezing in isopentane pre-cooled by liquid nitrogen. Cryostat sections (4.0 μm) were dried for 24 h and slides were fixed in acetone. The endogenous peroxidase activity was quenched by treatment with PBS containing 1.5% H₂O₂ for 5 minutes. Slides were stained for 1 h at room temperature with PBS containing Fas antibody (Research Diagnostics Inc., 0.7 μg/ml) diluted in the presence of 10% normal goat serum. The slides were microbed at room temperature for 30 min with rabbit secondary antibody conjugated to horseradish peroxidase (Jackson 1:100). DAB (Sigma Fast DAB tablets) was used as the chromagen and slides were counter-stained with hematoxylin. Additional liver samples were fixed in 10% buffered formalin, embedded in paraffin, sectioned (5 μm), and stained with hematoxylin and eosin (H&E). There was no evidence of significant liver toxicity and no histological differences among the saline, the LoxP control viral infected and the human RNase H-containing virus infected livers.

### Example 29

### Effect of Reduction of RNase H Levels on Antisense Potency

To complement the overexpression experiments, levels of human RNase H1 and H2 in cells have been reduced using potent selective DNA-like antisense oligonucleotides and double stranded oligoribonucleotides believed to work via an siRNA mechanism. This approach was taken because genetic knockouts of human RNase H1 are lethal (Busen, 1980, J. Biol. Chem., 255, 9434-9443; Cerritelli et al., 2003, Mol. Cell., 11, 807-815.

Double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

Oligonucleotides were identified by screening in cells (Crooke, S. T., 2003, in Burger's Medicinal Chemistry, 6^{th} ed., pp 115-166; Vickers et al., 2003, J. Biol. Chem. 278, 7108-7118).

Table 7 shows the most potent DNA-like antisense oligonucleotides and siRNA identified in the screens.

TABLE 7

Active antisense compounds against human RNase H
% Inhib is percent inhibition of RNase H enzyme levels
Bold residues are 2′-O-methoxyethyls. All 2′-MOE
cytosines are 5-methylcytosines
“H1” or “H2” in target refers to human RNase H1
(Genbank accession no. AF039652, provided here as
SEQ ID NO: 77) or human RNase H2 (Genbank accession
no. AY363912, SEQ ID NO: 11).
“Target position” refers to nucleotide position of the
5′-most nucleotide of the oligonucleotide target site
on the RNase H target nucleotide sequence.

| | | | | | | |
|---|---|---|---|---|---|---|
| | | SEQ | | | | |
| | | ID | | Target | Target | % |
| ISIS # | Sequence (antisense) | NO: | Target | position | region | Inhib |

DNA-like gapped oligonucleotides:

| | | | | | | |
|---|---|---|---|---|---|---|
| 18173 | GCGCTTAACACCGCACTTCC | 78 | H1 | 1 | 5′UTR | 30.1 |
| 18174 | CATCGCTCACTCCCGGCACC | 79 | H1 | 65 | Start | 51.4 |
| | | | | | codon | |
| 18177 | CCTCATCCTCTGTGGCAAAC | 80 | H1 | 261 | Coding | 71.9 |
| 18178 | CCTCCGATTCTTGTCCATGT | 81 | H1 | 339 | Coding | 64.6 |
| 18180 | CGCCCAGGAAGTCTAATGCC | 82 | H1 | 598 | Coding | 73.9 |
| 18182 | TCTTCCAACCTTGAACCCAG | 83 | H1 | 741 | Coding | 42.8 |
| 18184 | TGGCTCAAGTTCTCCCAAGG | 84 | H1 | 959 | 3′UTR | 78.9 |
| 18186 | TGCAGGCTATTTTCCACACC | 85 | H1 | 1006 | 3′UTR | 86.8 |
| 194178 | TGCAGGCTATTTTCCACACC | 85 | H1 | 1006 | 3′UTR | |
| 21955 | CAGTTTCTCCACGAATTGCC | 86 | H2 | 627 | Coding | 33.3 |
| 21956 | TTTTGTCTTGGGATCATTGG | 87 | H2 | 681 | Coding | 63.1 |
| 21957 | AGCTGAACCGGACAAACTGG | 88 | H2 | 742 | Coding | 68.1 |
| 21958 | CCTCTTTCTCCAGGATGGTC | 89 | H2 | 775 | Coding | 38.0 |
| 21959 | ACTCCAGGCCGCGTTCCAGG | 90 | H2 | 913 | Coding | 81.2 |
| 21960 | CCTACGTGTGGTTCTCCTTA | 91 | H2 | 1003 | 3′UTR | 91.7 |
| 21961 | GCACACTCCCACCTTGCTTC | 92 | H2 | 1041 | 3′UTR | 6.1 |
| 21962 | CAAAAGGAAGTAGCTGGACC | 93 | H2 | 1071 | 3′UTR | 36.0 |
| 194186 | CCTACGTGTGGTTCTCCTTA | 91 | H2 | 1003 | 3′UTR | |

Small interference RNA (siRNA)
(shown are oligoribonucleotide sense strands which are
annealed to their complementary antisense oligoribo-
nucleotide whose sequences correspond to above
sequences (± overhang)

| | | | | | | |
|---|---|---|---|---|---|---|
| Si-H1 | AAGUUUGCCACAGAGGAUGAG | 94 | H1 | 259 | Coding- | 89.9 |
| | | | | | sense | |
| Si-H1B | AAGCCGAGCGUGGAGCCGGCG | 95 | H1 | 436 | Coding- | 77.2 |
| | | | | | sense | |
| Si | GGCAAUUCGUGGAGAAACUGC | 96 | H2 | | Coding- | 19.2 |
| 21955 | | | | | sense | |
| Si | CCAAUGAUCCCAAGACAAAAG | 97 | H2 | | Coding- | 87.6 |
| 21956 | | | | | sense | |
| Si | CCAGUUUGUCCGGUUCAGCUG | 98 | H2 | | Coding- | 42.4 |
| 21957 | | | | | sense | |
| Si | GACCAUCCUGGAGAAAGAGGC | 99 | H2 | | Coding- | 45.8 |
| 21958 | | | | | sense | |
| Si | CCUGGAACGCGGCCUGGAGUC | 100 | H2 | | Coding- | 53.8 |
| 21959 | | | | | sense | |
| Si | UAAGGAGAACCACACGUAGGG | 101 | H2 | | 3′-UTR- | 9.9 |
| 21960 | | | | | sense | |
| Si | GAAGCAAGGUGGGAGUGUGCU | 102 | H2 | | 3′-UTR- | 15.2 |
| 21961 | | | | | sense | |
| Si | GGUCCAGCUACUUCCUUUUGG | 103 | H2 | | 3′-UTR- | 0 |
| 21962 | | | | | sense | |

Synthesis and purification of chimeric (gapped) 2′-O-methoxyethyl phosphorothioate oligonucleotides was as described in previous examples. Unmodified oligodeoxynucleotides were purchased from Invitrogen (Carlsbad Calif.).

As a general guide, nucleic acid duplexes comprising the antisense compounds of the present invention and their complements may be designed to target RNase H1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

Single-nucleotide or multiple overhangs may also be used, as may blunt-ended duplexes. Overhangs may be dTdT (as in the Tuschl canonical siRNAs) or may be complementary or identical to the target sequence at the same position, or may be other bases. The duplex may be unimolecular (e.g., a full or partial hairpin) or bimolecular) and may be fully or partially double-stranded.

RNA strands of the duplex can be synthesized by methods disclosed herein or purchased from Dharmacon Research Inc., (Lafayette, Colo.). Once synthesized, the complementary strands are annealed.

For this experiment oligoribonucleotides were purchased from Dharmacon Research, Inc. (Boulder Colo.). siRNA duplexes were formed in the solution containing 20 μM each oligoribonucleotide (sense strand shown and antisense strand complement—generally with a one base overhang at each end), 100 mM potassium acetate, 30 mM HEPES-KOH pH 7.4, 2 mM magnesium acetates. Reactions were heated for 1 minute at 90° C. and incubated for 1 hour at 37° C. The control gapped antisense oligonucleotide, is ISIS 29848, NNNNNNNNNNNNNNNNNNNN (SEQ ID NO: 104), where N is a mixture of A, G, C and T. Bold residues are 2′-MOE residues. The siRNA controls are either the single strand sense RNA strand or other RNA duplexes that are not complementary to the target. For transfection of cells, cells were incubated with a mixture of 3 μg/ml lipofectin (Invitrogen, Carlsbad Calif.) per 1-200 nM oligonucleotide or siRNA in OptiMEM medium (Invitrogen). After 4 hours the transfection mixture was aspirated from the cells and replaced with fresh medium containing 10% fetal bovine serum and the cells were incubated at 37° C. in 5% CO₂ until harvest or second transfection.

The most potent gapped oligonucleotide in inhibiting human RNase H1 was ISIS 18186, targeted to nucleotides 1006 to 1025 in the 3′ untranslated region of RNase H1 mRNA (Genbank accession no. AF039652). The most potent gapped oligonucleotide for inhibiting human RNase H2 is ISIS 21960, targeted to nucleotides 989 to 1008 in the 3′ untranslated region of human RNase H2 mRNA (Genbank accession no. AY 363912). The most potent siRNA for RNase H1 (si-H1) was targeted to nucleotides 259-279 in the coding region of the mRNA. The most potent siRNA for RNase H2 was si-21956 targeted to nucleotides 667-686 in the coding region.

The effects of various concentrations of each of the optimized inhibitors were then evaluated. Cells were treated with various amounts of gapped oligonucleotide or siRNA for 24 hours. Total RNA and cell lysates were prepared. RNA was subjected to 1.2% agarose/formaldehyde gel (5 μg total RNA/lane) and Northern blot analysis with a ³²P-labeled human RNase H1 or H2 or G3PDH cDNA probe. 20 μg protein of cell lysate was used for gel renaturation assay to test RNase H1 activity or for Western blotting with antibody to human RNase H2.

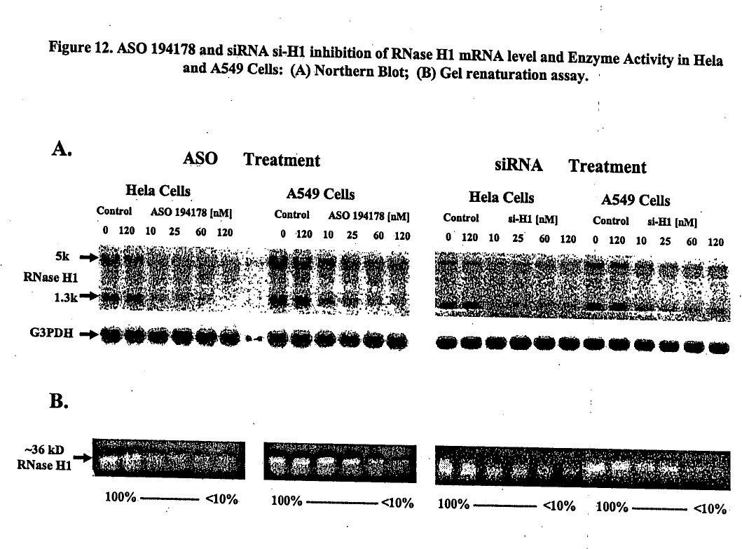

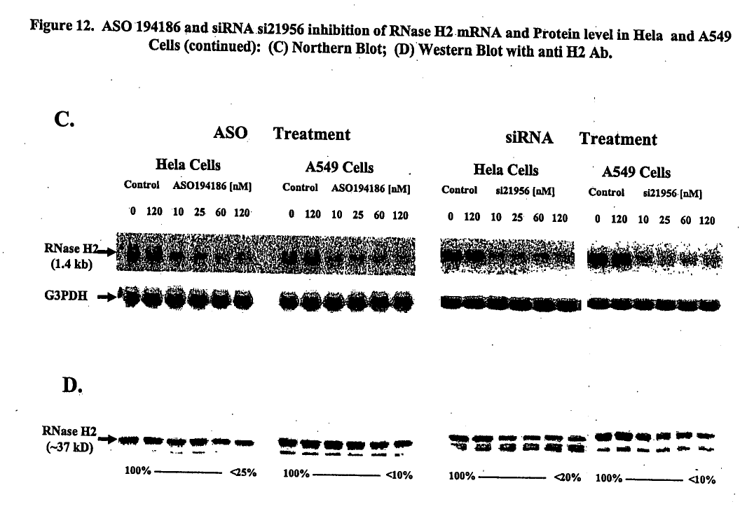

Both the gapped oligonucleotide and siRNA inhibitors resulted in potent dose dependent selective loss of RNase H1 RNA in both HeLa and A549 cells (FIG. 12A). Both the 1.3 kb band and the 5 kb band thought to be preprocessed human RNase H1 RNA were reduced. Further, the RNase H1 activity in both cell lines was reduced as shown in the gel renatuation assay (FIG. 12B). There were no effects of these RNase H1 inhibitors on RNase H2 levels. The duration of effect for both the ASO and siRNAs was greater than 48 hours. FIGS. 12C and D show that both the ASO and siRNAs targeting human RNase H2 mRNA reduced RNase H2 RNA and protein levels in a fashion comparable to that observed for RNase H1. Again, the effects were specific to RNase H2 and the duration of effect was greater than 48 hours.

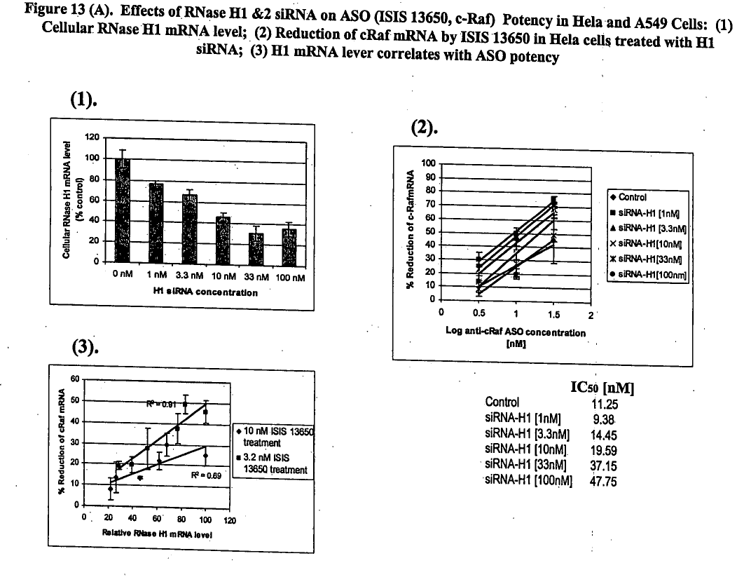

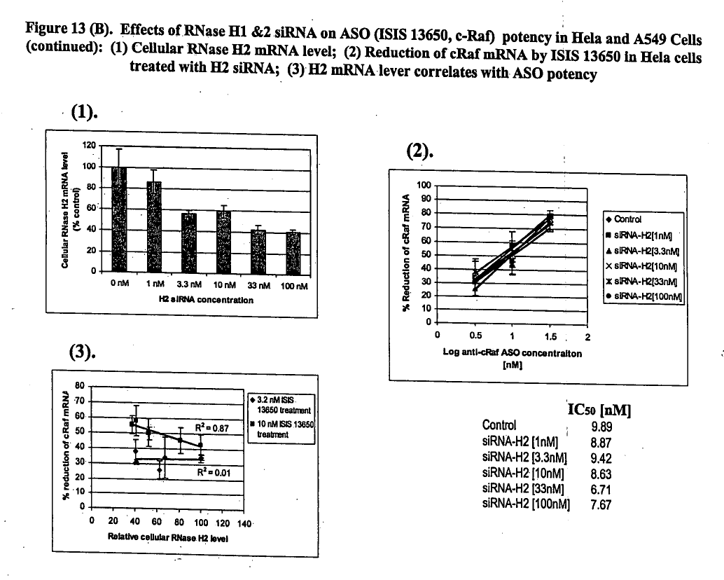

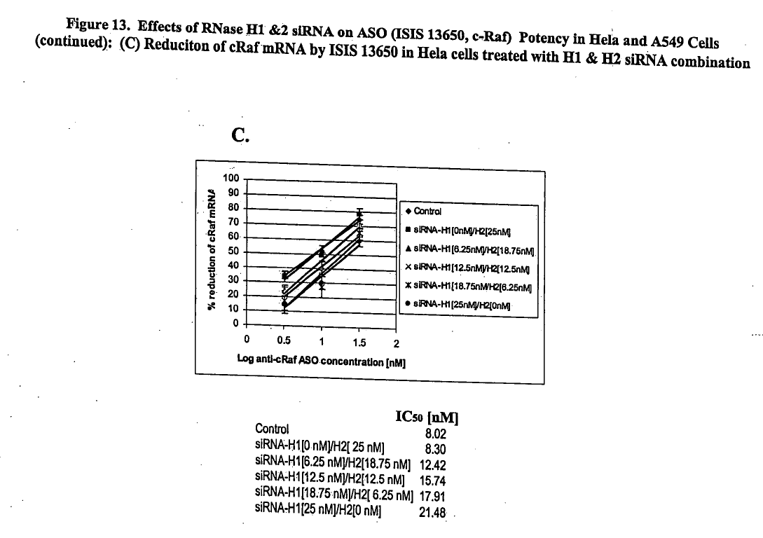

FIG. 13 shows that reduction in the levels of human RNase H1 but not RNase H2 reduced the potency of gapped oligonucleotides targeting c-Raf mRNA in both HeLa and A549 cells. Cells were first transfected with various concentrations of RNase H1 siRNA as indicated for 10 hours before the cells were split into 96 well format cell culture plates (6000 cells/well) and incubated for 10-14 hours. The cells were transfected with various concentrations of ISIS 13650 for 24 hours before harvest. Total RNAs were prepared and the cellular c-Raf and RNase H1 mRNA levels were determined with RT-PCR in which the reverse transcription and PCR amplification of c-Raf and RNase H1 mRNAs were performed in the 96 well format with the primer sets described in previous examples. The vertical bars in FIG. 13A represent standard error of the mean of 3-6 replicates of a representative experiment.

Increasing concentrations of the siRNA to human RNase H1 resulted in a comparable reduction in the potency of the c-Raf antisense oligonucleotide. Further, there is a clear correlation between the reduction of c-Raf mRNA by the c-Raf oligonucleotide and the cellular level of human RNase H1 (R²=0.91 or 0.69; p<0.01). See FIG. 13A. FIG. 13A1 shows reduction of cellular RNase H1 by H1 siRNA. FIG. 13A2 shows effects of RNase H1 siRNA on the potency of c-Raf antisense oligonucleotide ISIS 13650. The IC50s are given under the graph. FIG. 13A3 shows a correlation of cellular RNase H1 mRNA levels with the potency of ISIS 13650. Cellular RNase H mRNA levels were determined by RT-PCR. The RNase H1 mRNA levels in arbitrary units for untreated cells were divided by level or the RNase H1 mRNA from treated cells to obtain the relative level of RNase H1 RNA.

Surprisingly, an extrapolation of the c-Raf 3.2 nM or 10 nM dose response curves would not demonstrate zero antisense activity when there was no human RNase H1 mRNA.

In contrast, in experiments conducted similarly, the siRNA to human RNase H2 reduced the cellular RNase H2 RNA equivalently to levels observed for RNase H1 but there was no effect on the potency of the c-Raf oligonucleotide. Nor was there a correlation between cellular RNase H2 RNA levels and the potency of the c-Raf antisense oligonucleotide (FIG. 13B).

To further confirm that inhibition of RNase H1 and not RNase H2 caused a loss of antisense oligonucleotide potency, the experiment shown in FIG. 13C was performed. In this experiment, the total siRNA concentration was held constant at 25 nM and the ratio of RNase H1 siRNA to RNase H2 siRNA was varied from zero to one. The vertical bars represent the standard error of the mean of three replicates of a representative experiment. 25 nM siRNA to human RNase H2 had no effect on the potency of the c-Raf antisense oligonucleotide. As the ratio of RNase H1 siRNA versus RNase H2 siRNA was increased, the c-Raf antisense oligonucleotide progressively lost potency. Similar results were observed in A549 cells (FIG. 7C, 150 nM of siRNA to RNase H1 or H2) and for a number of other antisense oligonucleotides to other cellular targets. Vertical bars represent standard error of the mean of six replicates of a representative experiment.

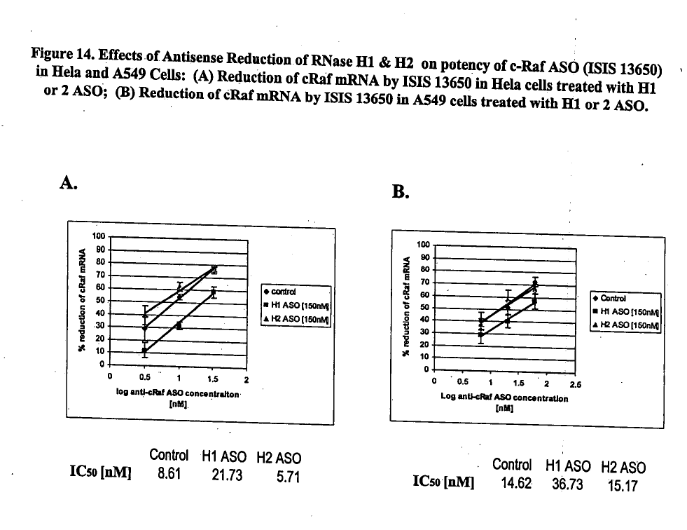

FIG. 14 shows that an antisense oligonucleotide targeted to human RNase H1 but not to human RNase H2 reduced the potency of the c-Raf antisense oligonucleotide in HeLa (panel A) and A549 cells (panel B). Each RNase H antisense oligonucleotide was transfected at 150 nM concentration. Vertical bars represent standard error of the mean of six replicates of a representative experiment. These results were entirely comparable to the effects of siRNAs to RNases H1 and H2.

### Example 30

### Additional RNase H Activities in Human Cells

The RNase H1 inhibition experiments described in previous examples showed that the c-Raf antisense oligonucleotide was active even when cellular RNase H1 levels were reduced by more than 90%. Furthermore, the RNase H1 inhibition curves did not extrapolate to zero activity at zero RNase H1.

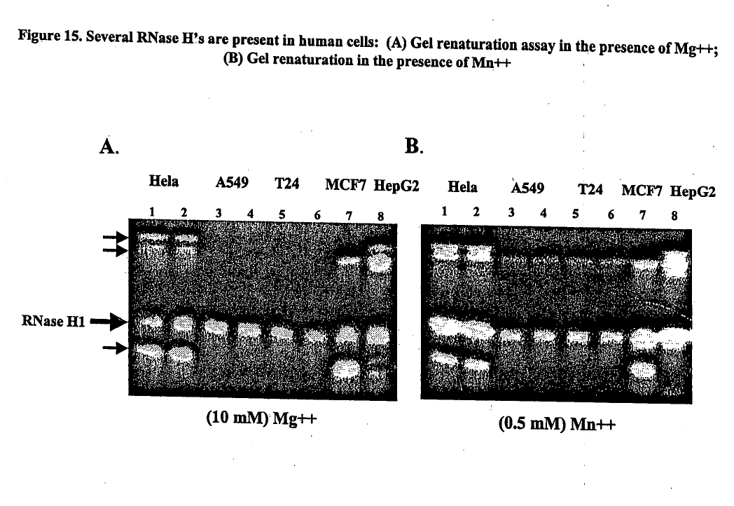

Several higher molecular weight (apparent size 60-70 kD) bands as well as several lower molecular weight bands from cell homogenates were observed in the gel renaturation assay (FIG. 15). It should be noted that molecular weight determination on gel renaturation assays is by nature imprecise. By definition the observed bands are RNase H bands because they cleave the RNA/DNA substrate in the gel matrix. Cell lysates were prepared in RIPA lysis buffer from human HeLa, A549, T24, MCF7 and HepG2 cells as described in previous examples. 20 μg protein from each lysate was used in the gel renaturation assay. Lanes 1-2: HeLa cell lysate; Lanes 3-4: A549 lysate; Lanes 5-6: T24 cell lysate; Lane 7:MCF7 lysate; Lane 8:HepG2 lysate. The lysates from lanes 2, 4 and 6 were prepared with the lysis buffer without phosphatase inhibitors. Panel 15A: Gel renaturation assay in the presence of 10 mM Mg²⁺; Panel 15B: Gel renaturation in the presence of 0.5 mM Mn²⁺.

The higher molecular weight RNase H bands were observed in several cell types when the renaturation assay was performed under standard conditions (10 mM Mg²⁺). The level of activity and the number of extra bands varied from cell type to cell type and from one cell preparation to another (FIG. 15A). When the assay was performed in the presence of 0.5 mM Mn²⁺, the extra RNase H activity bands were more apparent and at least one higher molecular weight activity band was observed in all cell lines.

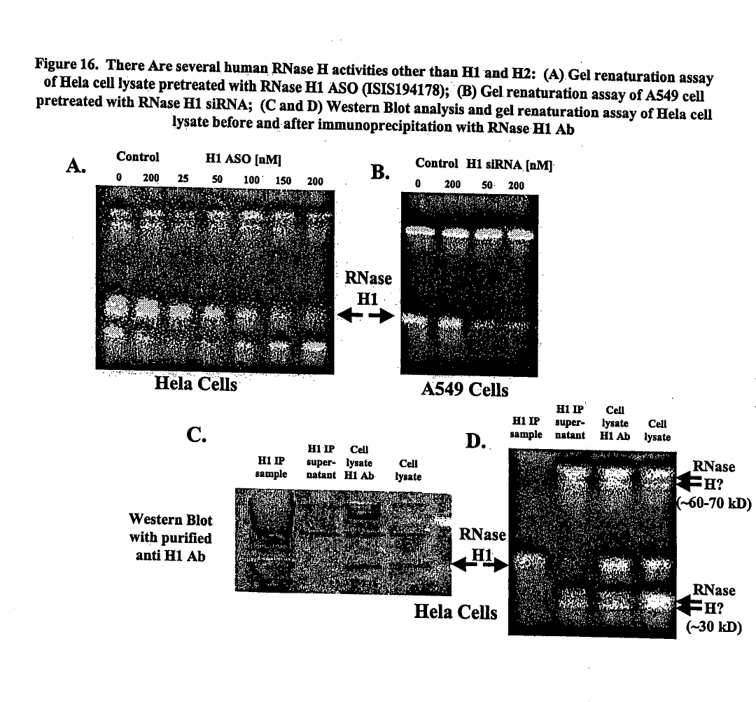

Reduction of RNase H1 with either a gapped antisense oligonucleotide or an siRNA oligonucleotide in both HeLa (FIG. 16A) and A549 (FIG. 16B) cells reduced the RNase H1 band of activity in a gel renaturation assay (+Mg²⁺) and had no effect on the higher molecular weight bands of RNase H activity. Prior to preparation of the lysates, cells were treated with either a control antisense oligonucleotide (ISIS 29848, NNNNNNNNNNNNNNNNNNN where N=an equal mixture of A, C, T and G; SEQ ID NO: 104) or the RNase H1 antisense oligonucleotide ISIS 194178 at the concentrations indicated. FIG. 16C shows the results of quantitative immunoprecipitation of RNase H1 from cell lysates, using purified polyclonal antibodies to human RNase H1. The supernatant was separated from the protein A beads by centrifugation. All samples were then subjected to SDS-PAGE and probed with the purified human RNase H1 antibody. The supernatant after immunoprecipitation of RNase H1 contained no detectable RNase H1. Further, in the gel renaturation assay (FIG. 16D) there was no RNase H1 activity in the immunoprecipitation supernatant. Nevertheless, in the immunoprecipitation supernatant, several of the novel RNase H activity bands remained (FIG. 16D). Similar results are obtained when immunoprecipitation is done using the human RNase H2 antibody.

These results indicate that there are several previously unidentified RNases H in human cells that are not the RNase H1 or RNase H2 previously defined herein and by others, yet are active in a gel renaturation assay. Neither inhibition at the RNA level with antisense (DNA-like oligonucleotides or siRNA) to RNase H1 or RNase H2 nor precipitation with RNase H1 or H2 antibodies affected the level or activity of the novel RNases H.

The RNase H band with apparent size of 60-70 kDa (on gel renaturation) is substantially isolated and purified by preparative SDS-PAGE, concentration on Vandekerckhove gel, blotting onto nitrocellulose, and Coomassie blue staining as described in Frank et al., 1998, Proc. Natl. Acad. Sci. 95, 12872-12877. A renaturation gel is used to confirm RNase H activity of the concentrated band. The RNase H band is excised from the membrane and digested with sequencing grade trypsin. Peptide mapping is carried out as described by Frank (ibid.) and peptide fractions are analyzed by automated Edman degradation. Based on the peptide fragment sequences, corresponding human expressed sequence tags (ESTs) may be identified in the EST database (National Center for Biotechnology Information) using the BLAST algorithm. Altschul et al., 1990, J. Mol. Biol. 215, 403-410; Altschul, et al., 1997, Nucleic Acids Res. 25, 3389-3402. These EST sequences are used to design primers for cloning of the novel RNase H cDNA. Alternatively, sets of degenerate probes may be designed based on the protein fragment sequences, and used directly to probe libraries of human cells for cDNA sequences corresponding to the novel RNase H. The full length cDNA encoding the novel 60-70 kDa human RNase H is expressed and purified as described for human RNase H1 and H2 in examples above.

### Example 31

### Purification of Additional Proteins with RNase H Activity from Human Cells

Purification of the higher molecular weight RNase H band (apparent molecular mass larger than RNase H1) shown in FIGS. 15 and 16 was performed by preparative gel electrophoresis. HeLa cell lysates were prepared in 0.5% triton X-100 RIPA lysis buffer and fractionated by electrophoresis on a 10% SDS-polyacrylamide preparative gel (Bio-Rad, Hercules Calif.). A sample of each fraction was loaded onto a 4-20% gradient SDS-polyacrylamide gel and electrophoresed. Another sample of each fraction was loaded onto an acrylamide gel for the gel renaturation assay described in previous examples. This allows correlation of RNase H activity with molecular mass of the enzyme. In addition to the RNase H1 band, a higher molecular weight band of RNase H activity was observed having a molecular mass of roughly 50-70 kD. Note that molecular mass estimates in the renaturing gel assay are imprecise due to conditions and inability to use accurate size standards. The band originally estimated as being 60-70 kD in size was subsequently found to be closer to 50 kD in size.

The roughly 50-kDa fraction with confirmed RNase H activity from preparative gel electrophoresis was further fractionated by reverse-phase chromatography with C-5 column (Supelco, Bellefonte Pa.). The fractions after HPLC were tested for their RNase activities by gel renaturation assay. The fraction containing the highest RNAse activity was enzymatically digested with trypsin protease. An aliquot estimated to contain 5 μg total protein and corresponding to 1/40^{th} of the most active fraction was diluted with 100 mM ammonium bicarbonate buffer (pH 8.5) to 50 uL. 2 μL DTT, dissolved in 100 mM ammonium bicarbonate, was added to a final concentration of 2 mM, and the sample was incubated at 56° C. for 1 hour. After cooling, 2 μL iodoacetamide, dissolved in 100 mM ammonium bicarbonate, was added to a final concentration of 18.5 mM. The sample was incubated at room temperature in the dark for 3 hours. Modified trypsin (Promega, Madison Wis.) at 0.5 μg/μL was diluted five-fold with 100 mM ammonium bicarbonate. A 2.5 μL aliquot (0.25 μg) was added to give an enzyme-to-substrate ratio of 1:20. The sample was then incubated at 37° C. for 13 hours. Digestion was quenched by addition of 2 μL glacial acetic acid to give a pH of ˜3.

The digested sample was analyzed by nanoflow reversed-phase HPLC/micro electrospray ionization/MS/MS (Martin, S. E. et al., 2000, Anal. Chem. 72: 4266-4274). An aliquot of 3 μL of the digest (corresponding to 1/17^{th} of the digested material, or 6 pmol total protein) was loaded onto an in-house fabricated microHPLC trapping column packed in 360 μm o.d., 75 μm i.d. fused silica (Polymicro Technologies, Phoenix, Ariz.) to a length of 3 cm with 5 μm C₁₈ beads (100 Å pore size Monitor C₁₈, Column Engineering, Ontario, Calif.). The sample was washed and desalted with HPLC solvent A (see below) for 10 minutes prior to connecting the resolving column and electrospray emitter tip. Reverse phase HPLC (Agilent 1100 Quaternary pump) was performed using a binary solvent system comprised of 0.1M acetic acid (Aldrich, 99.99% purity) in water (solvent A) and 0.1M acetic acid, 70% acetonitrile (HPLC grade, Honeywell Burdick and Jackson, Muskegon Mich.) in water (solvent B). The gradient program was 0-40% B in 50 minutes, 40-100% B in 10 minutes, hold at 100% B 1 min, 100-0% B in 5 minutes. The resolving column was fabricated in-house from 360 μm o.d., 50 μm i.d. fused silica packed to a length of 8 cm with 5 μm C₁₈ beads. The electrospray emitter tip (360 μm o.d. by 20 μm i.d. shaft, 5 μm i.d. tip, New Objective, Woburn, Mass.) was attached using a short section of 0.012″ i.d. teflon tubing (Upchurch Scientific, Oak Harbor, Wash.). Peptides were eluted at a measured flow rate of 45 nL/min into the inlet of a linear ion trap mass spectrometer (LTQ, Thermo, San Jose, Calif.). MS and MS/MS were performed in an automated fashion using the data-dependent MS/MS capability of the instrument control software (Xcalibur, Thermo, Schaumburg Ill.) as described (Mosammaparast, N. et al., 2001, J. Cell Biol. 153: 251-262; Ficarro, S. B. et al., 2002, Nat. Biotechnol. 20: 301-305). Briefly, an MS survey scan was acquired over the mass-to-charge range 300 to 2000. The 5 highest-abundance ions were identified and selected as precursors for five subsequent MS/MS scans. Following the MS/MS scans, these precursors were placed on a dynamic exclusion list for 45 seconds, which prevents multiple MS/MS scans on the same peptide. Another MS survey scan was then acquired and the sequence was repeated. Typically, a single cycle of MS scan and 5 MS/MS scans took 2 seconds. Data was acquired for 74 minutes, during which time 14,000 spectra were acquired. Post-acquisition data processing was performed using the Bioworks Browser software suite (Thermo). MS/MS data were extracted and compared with a database of human proteins. This database was downloaded from the National Center for Biotechnology Information (NCBI, http://www.ncbi.nih.gov) on Jan. 8, 2004 and contains 200,000 proteins. Data analysis was performed using the SEQUEST algorithm (Eng, J. et al., 1994, J. Am. Soc. Mass Spectrom. 5: 976-989; Yates J R et al., 1995, Anal Chem. 67:1426-36). This algorithm uses correlation scoring to compare MS/MS data with theoretical spectra generated from database peptide sequences, subject to known properties of peptide fragmentation by low-energy collision-induced dissociation and enzymatic digestion specificity. Peptide sequences achieving a cross-correlation score of 3.0 or greater were deemed to be correctly assigned.

A total of 117 proteins were identified by 1 or more peptides using the criteria stated above, including 7 proteins for which 10 or more peptides were identified. Among these was flap structure-specific endonuclease 1, (Fen1), NCBI gi number 4758356. This 380-amino acid (calculated molecular weight approximately 42 kDa) protein cleaves DNA flap strands that terminate with a 5′ single-stranded end and is known to remove 5′ overhanging flaps in DNA repair and process the 5′ ends of Okazaki fragments in lagging strand DNA synthesis. Rumbaugh et al., 1999, J. Biol. Chem., 274, 14602-14608.

To determine whether Fen1 was actually responsible for the RNase H activity seen on the gel renaturation assay, an immunoprecipitation of HeLa lysate was conducted using an antibody to Fen1 (Abcam Co., Cambridge Mass.), and the immunoprecipitated protein was tested in the gel renaturation assay for RNase H activity. The immunoprecipitated Fen1 enzyme was found to have RNase H activity band at the expected molecular mass position of approximately 50 kD.

A human Fen1 cDNA clone was purchased from Invitrogen, (Carlsbad Calif.). The cDNA encoding the 380 amino acid full length Fen1 protein was amplified by PCR with engineered his-tag either on the N-terminus or C-terminus and cloned into into a pET3a expression vector. Three clones were shown to express a band of the correct size on Western blot when probed with antibody to Fen1. E. coli lysates of these three clones were run in the RNase H gel renaturation assay and the C-terminal his-tag fued Fen1 was shown to have RNase activity at the appropriate size position. Thus it is believed that human Fen1 accounts for some if not all of the higher molecular weight band showing RNase H activity in the gel renaturation assay (see FIGS. 15 and 16).

Using the standard RNase H cleavage assay (see previous examples), it was confirmed that the expressed Fen1 with C-terminal his-tag cleaves the RNA strand of DNA/RNA duplexes. It was found that this enzyme is capable of cleaving both an unmodified DNA/RNA duplex and a gapmer/RNA duplex in which the oligonucleotide (“DNA”) strand of the duplex is a chimeric oligonucleotide with 2′-O-methoxyethyl flanks and a 2′-deoxynucleotide center gap.

Purification of the lower molecular weight RNase H band (apparent molecular mass smaller than RNase H1) shown in FIGS. 15 and 16 was performed similarly by preparative gel electrophoresis. HeLa cell lysates were prepared in 0.5% triton X-100 RIPA lysis buffer and fractionated by electrophoresis on a SDS-polyacrylamide preparative gel (Bio-Rad, Hercules Calif.). A sample of each fraction was loaded onto a gradient SDS-polyacrylamide gel and electrophoresed. Another sample of each fraction was loaded onto an acrylamide gel for the gel renaturation assay described in previous examples. This allows correlation of RNase H activity with molecular mass of the enzyme. In addition to the RNase H1 band, a lower molecular weight band of RNase H activity was observed having a molecular mass of roughly 30 kD (Note that molecular mass estimates in the renaturing gel assay are imprecise due to conditions and inability to use accurate size standards). This band was found not to react with an antibody to human RNase H1 so is believed not to be a degradation product of that enzyme.

### Example 32

### Roles of Human RNase H2 in Cell Cycle, DNA Replication and Repair

The lack of activity of expressed or purified RNase H2 suggested that protein partners, such as those in a DNA replication complex, may be necessary for RNase H2 activity (Wu et al., 2004). Experiments were done to identify proteins that co-precipitate with RNase H2.

PCNA, Fen-1, and RNase H₂Co-Immunoprecipitate—To identify partners that might regulate RNase H2, polyclonal antibodies directed against RNase H2 were used to precipitate proteins from HeLa cells. Goat Fen-1 polyclonal antibody (pAb), was purchased from Santa Cruz Biotechnology (Santa Cruz, Calif.). Mouse PCNA monoclonal antibody (mAb) was purchased from Upstate Biotechnology (Lake Placid, N.Y.). Donkey anti-goat IgG peroxidase-conjugated secondary antibody was purchased from Jackson ImmunoResearch Laboratories (West Grove, Pa.). Rabbit anti-mouse IgG peroxidase conjugated secondary antibodies were purchased from Sigma (St. Louis, Mo.). Rabbit RNase H1 and RNase H2 pAb were developed by Wu et al., (Wu et al., 2004). 5×10⁶ HeLa cells were isolated by centrifugation at 1100×g for 2 min, and washed twice with PBS. The washed cells were incubated with lysis buffer (50 mM Tris pH 7.8, 120 mM NaCl, 0.5% NP-40) supplemented with 1/100× volume Halt protease inhibitor (Pierce Chemical Co., Rockford Ill.) on ice for 5 min. The lysates were centrifuged at 16,000×g for 2 min, and pelleted material was discarded. 1 μg antibody was added to the lysate supernatant and they were mixed together on a rocker for 1 h at 4° C. 30 μl Protein A-sepharose bead slurry in 50 mM Tris pH 7.6, 20 mM NaP was added and mixing continued at 4° C. for 30 min. Beads were isolated through microcentrifugation and washed; three times with chilled lysis buffer. 2× sodium dodecyl sulfate-polyacrylamide gel electrophoresis (SDS-PAGE) sample buffer (0.05% bromophenol blue, 0.0625M Tris-HCl pH 7.6, 1% SDS, 10% glycerol, 1% β-mercaptoethanol) was heated at 100° C. with the beads for 5 min to elute material from the lysate that associated with the antibody. After microcentrifugation, the supernatant was subjected to size-separation by SDS-PAGE in a 4-20% acrylamide gel (Tris-Glycine, Invitrogen, Carlsbad Calif.). Separated proteins were transferred from the polyacrylamide gel to a nitrocellulose membrane at 44 mA for 2 h in Western transfer buffer (Invitrogen). Membranes were blocked by rocking for 1 h at 22° C. in 50 ml blocking buffer (5% powdered milk in TNT [20 mM Tris-Cl (pH 7.6), 137 mM NaCl, 0.1% Tween 20]). The membranes were then rocked for 1 h at 22° C. in 10 ml blocking buffer with 1 μg of primary antibody. The membranes were washed twice with 50 ml TNT; each time for 10 min while rocking at 22° C. Next the membranes were incubated in 10 ml blocking buffer with 1.5 μg of secondary antibody for 30 min at 22° C. Lastly, the membranes were washed twice with 50 ml TNT while rocking at 22° C. and incubated with ECL+Plus chemiluminescence reagents (Amersham Biosciences, Piscataway N.J.). Luminescence was visualized using a Phosphorimager Storm 860 (Molecular Dynamics; Sunnyvale, Calif.).

Fen-1 and PCNA were detected in RNase H2 immunoprecipitates Neither Fen-1 or PCNA was detected in immunoprecipitates generated with antibodies against RNase H1. Immunoprecipitates isolated using antibodies against Fen-1 and PCNA were incubated with a radiolabeled heteroduplex RNase H substrate, but products resulting from nucleolytic cleavage were not detected.

Expression of RNase H2 is Regulated During the Cell Cycle—RNA was harvested from HeLa cells at various times during a single cell cycle and probed equivalent amounts of RNA were probed with an oligoribonucleotide complementary to either RNase H2 or G3PDH. HeLa (human cervical carcinoma) cells were obtained from American Type Culture Collection (Manassas, Va.) and were grown in Dulbecco's modified eagle's medium (DMEM) from Invitrogen (Carlsbad, Calif.) with 10% fetal bovine serum (FBS; Sigma), streptomycin (0.1 mg/ml), and penicillin (10 U/ml) (PS; Invitrogen) (DMEM FBS/PS). All washes of HeLa cells in tissue culture plates were performed with 2.5 ml phosphate-buffered saline (PBS, Invitrogen). HeLa cells were cultured in all cases at 37° C. in air supplemented with 5% CO₂. HeLa cells were synchronized at early S-phase using the double thymidine method (Johnson et al., 1993). 2.5×10⁶ HeLa cells were grown in 10 cm tissue culture dishes in DMEM FBS/PS. The cells were washed two times, and growth medium was changed to DMEM FBS/PS+2 mM Thymidine (Sigma, St. Louis Mo.) followed by growth for 17 h. The cells were washed two times again, the growth medium was replaced with DMEM FBS/PS, and culture continued for 9 h. Subsequently the cells were washed two times, growth medium was replaced with DMEM FBS/PS+2 mM Thymidine, and culture continued for 17 h. Cells were washed two times after this final 2 mM Thymidine treatment, growth medium was replaced with DMEM FBS/PS, and samples of the synchronized population of HeLa cells were taken at various times during the 24 h cell cycle. Total RNA was isolated from HeLa cells using RNeasy kits (Qiagen). 15 mg of total RNA was separated on a 1.2% agarose/formaldehyde gel and transferred to Hybond-N+ (Amersham Pharmacia Biotech) followed by UV cross-linking in a UV stratalinker 2400 (Stratagene; La Jolla, Calif.). To detect RNase H2 mRNA, hybridization was performed by using ³²P-labeled human RNase H2 cDNA probe in Quik-Hyb buffer (Stratagene) at 68° C. for 2 h. After hybridization, membranes were washed twice in 0.3M NaCl, 30 mM NaCitrate, 0.1% SDS at 22° C. for 15 min followed by another wash in 15 mM NaCl, 1.5 mM NaCitrate, 0.1% SDS at 60° C. for 30 min. Autoradiography from these membranes was analyzed using PhosphorImager Storm 860.

As expected for an enzyme with activity fundamental to genomic DNA synthesis, RNase H2 mRNA was found to be most abundant during S-phase of the cell cycle. In addition, HeLa cell lysates from various times during a single cell cycle were analyzed for the presence of the RNase H2 protein via Western blotting. In the same manner that RNase H2 mRNA abundance varied during the progression of a single HeLa cell cycle so did the abundance of the RNase H2 protein vary.

siRNA Reduction of RNase H2 Inhibits Progression Through the Cell Cycle—The distribution of unsynchronized HeLa cells in various stages of the cell cycle were measured 20 h after treatment with siRNA against RNase H1 or RNase H2. In preparation for transfection, 2.5×10⁶ HeLa cells grown in 10 cm tissue culture dishes were washed prior to growth in 2 ml of Opti-MEM (Invitrogen) for 30 min. 2.5 ml Opti-MEM was incubated with 3 μg/ml lipofection (Invitrogen) at room temperature for 15 minutes. After this period 100 nM, 50 nM, or 10 nM dsRNA with two 5′ adenines, but otherwise homologous to RNase H1 (sense strand 5′-AAG UUU GCC ACA GAG GAU GAG-3′; SEQ ID NO: 94) or RNase H2 (sense strand 5′-AAC CAA UGA UCC CAA GAC AAA-3′; SEQ ID NO: 105), was added. When transfected into HeLa cells at a concentration of 100 nM, these siRNA species were previously shown to inhibit the production of RNases H1 and H2 transcripts by 76±7% and 75±4% respectively (Wu et al., 2004). HeLa cells were incubated for 4 h with these transfection mixtures, after which the medium was replaced with DMEM FBS/PS. Samples of these cells were collected 20 h later for analysis. Reduction of RNase H1 message by siRNA treatment had no measurable effect on the distribution of HeLa cells in G1, S, or G2/M phases. In contrast, reduction of the RNase H2 message by treatment with siRNA decreased the abundance of HeLa cells in S and G2/M phases. There was a corresponding increase in the abundance of cells in G1 phase.

DNA Repair Relies Upon RNase H2 Activity—As DNA synthesis occurs not only during the genomic replication, experiments were conducted to test whether RNase H2 expression was important for the repair of DNA damage induced by UV-irradiation. HeLa cells, lipofected 20 h prior with siRNA that depleted levels of RNase H1 and RNase H2 mRNA by 76±7% and 75±4% respectively, were UV-irradiated and cultured for 2 h to allow the repair of their damaged DNA. Subsequently, the cells were embedded within agarose and subjected to single-cell electrophoresis followed by the staining of their nucleic acid with syber green. 5×10⁵ HeLa cells growing in 9.5 cm² tissue culture plates were washed twice with 2 ml PBS, overlain with 0.5 ml PBS, and exposed to 4 J/m² UV radiation in a UV Stratalinker 2400 (Stratagene). PBS was immediately removed from the cells following irradiation, and replaced with 4 ml DMEM FBS/PS. Cells were grown for 2 h to allow DNA damage to be repaired. Subsequently the cells were harvested by washing with PBS and incubating with 0.5 ml trypsin-EDTA for 30 sec at 37° C. Harvested cells were diluted in 5 ml DMEM FBS/PS, centrifuged at 300×g, and washed once with 5 ml PBS. Centrifugation at 300×g isolated the cells again, 1,000 cells were mixed with 150 μmelted 37° C. LM Agar (Trevigen; Gaithersberg, Md.), and placed upon a slide for microscopic observation. The slide was immersed in 4 C lysis solution (Trevigen) for 1 h, and then in alkaline solution (Trevigen) at 22° C. for 1 h. Slides were then immersed in 1×TBE twice for 5 min each, and subjected to 1 V/cm in 1×TBE for 15 min. The slides were then immersed in 70% ethanol twice for 5 min each time, and dried at 22° C. Dried slides were overlain with 1/1000 syber green (Trevigen) to stain DNA. Stained DNA was visualized using a Nikon Eclipse TE300 fluorescent microscope with a FITC filter. The intensity of syber green staining in the migrated DNA tails (% TailDNA) and the extent of their migration (TailLength/TotalLength) were quantified. Tail moments were calculated by the following formula:
% TailDNA(TailLength/TotalLength).
Levels of DNA damage remaining in the cells that had received either siRNA treatment were compared to the DNA damage remaining in HeLa cells that were not treated with siRNA against RNases H1 or H2. Reduction of RNase H2 was found to significantly increase the DNA mobility; a hallmark of DNA damage, while depletion of RNase H1 had no effect. Thus it is concluded that in much the same manner that RNase H1 depletion had little effect upon either cell cycle phase distribution in a population of HeLa cells or the abundance of Okazaki fragments in those cells, its depletion had no measurable effect upon the efficiency of DNA repair by HeLa cells. Again, the depletion of RNase H2 significantly inhibited DNA repair.

RNase H2 is the Nuclease Responsible for the Removal of Okazaki Fragment Primers from Human Genomic DNA—To characterize the importance of each RNase H isoform in the maturation of Okazaki fragments, the abundance of Okazaki fragments in human cells treated with siRNA against RNase H1 or RNase H2 was quantitated. DNA was isolated from HeLa cells that had been treated 24 h previously with siRNA against RNase H1 or RNase H2. 10×10⁶ cells were incubated in 2 ml lysis buffer [20 mM Tris pH 7.6, 0.5M NaCl, 0.5% NP-40, 100 U/ml Super RnaseIN (Ambion), 100 g/ml Proteinase K (Qiagen, San Diego Calif.)] for 1 h at 55 C. After they cooled to room temperature, 1.2 ml isopropanol was added to the lysates. Precipitated material was collected via centrifugation for 5 min at 1000×g, suspended in 1 ml TE (10 mM Tris pH 7.8, 1 mM EDTA) and extracted with an equal volume first of 1:1 Tris pH 7.8-buffered phenol:chloroform, then of chloroform alone. Aqueous-phases from these two extractions were diluted to 5 ml with TE to which 1 g/ml CsCl, 1 mg/ml ethidium bromide, and 100 U/ml Super RNaseIN were added. This solution was subjected to centrifugation at 100,000×g for 12 h, which resulted in the concentration of genomic DNA in a discrete ethidium bromide-stained band. Ethidium bromide was removed from the isolated DNA by extraction with 3× volume CsCl-saturated isopropanol, and DNA in the solution was precipitated by adding ⅙ volume 10M sodium acetate and 3.5× volume cold 100% ethanol. After 4° C. centrifugation for 5 min at 16,000×g, the precipitate was washed with cold 70% ethanol and centrifuged again under the same conditions. Genomic DNA thus purified was suspended in 300 μl TE supplemented with 100 U/ml Super RnaseIN, and sonicated for 5 min. A 1 μg/μl solution of genomic DNA was prepared from the final sonicated product. Vaccinia virus guanylyltransferase was utilized along with [³²P]-GTP in a 5′ RNA capping reaction to measure the relative abundance of unjoined Okazaki fragments in the genomic material. 2 μg genomic DNA were heated at 100° C. for 2 min to denature the DNA, and cooled in ice for 2 min to prevent reannealing of complementary strands. 10 μl capping reactions were prepared containing the denatured genomic material, 50 mM Tris pH 7.9, 6 mM KCl, 2.5 mM DTT, 1.25 mM MgCl₂, 0.1 mg/ml BSA, 0.33 mM S-adenosyl methionine, 10M [³²P]-GTP (800 Ci/mmol, Amersham; Sunneyvale, Calif.), and 5 U vaccinia virus guanylyltransferase (Ambion; Austin, Tex.). The reactions were incubated for 1 h at 37° C. Following incubation, unincorporated nucleotides were separated from the genomic material by centrifugation at 1100×g through a sephadex G-50 column (Roche, Switzerland). Nucleic acids collected from the column were precipitated with 9 μg glycoblue (Ambion), ⅙× volume 10M sodium acetate, and 3.5× volume cold ethanol. Centrifugation for 5 min at 4° C., 16,000×g collected the genomic nucleic acid as a precipitate that was subsequently washed with cold 70% ethanol and centrifuged again for 5 min at 4° C., 16,000×g before drying. The precipitate was suspended in denaturing solution (4M urea, 20 mM EDTA) and heated at 100° C. for 3 min followed by rapid cooling on ice for 2 min. Denatured samples were loaded onto 8% acrylamide 4M urea gels for electrophoresis followed by autoradiography to determine the size of nucleic acid chains that had been labeled with a 5′ 7-methylguanosine cap containing ³²P.

After treatment with siRNA against RNase H2, Okazaki fragments measured as 182±10% more abundant than in untreated HeLa cells. Treatment with siRNA against RNase H1 did not lead to any increase in Okazaki fragment abundance. This demonstrates the fundamental importance of RNase H2 for the removal of RNA primers from the 5′ end of lagging strand Okazaki fragments. No such accumulation of unprocessed Okazaki fragments was seen upon treatment of cells with siRNA against RNase H1. Apparently each Okazaki fragment generated during lagging strand DNA synthesis relies upon the RNase H2, but not the RNase H1, nuclease to remove its 5′ ribo-nucleotide primer.

These results regarding the importance of RNase H2 for both S-phase progression and DNA repair represent the first demonstrations of RNase H isoform activity in either genomic or repair DNA replication in whole cells.

1. A substantially isolated and purified human RNase H which has an apparent molecular weight of 60-70 kDa in a gel renaturation assay and which cleaves the RNA strand of an RNA-DNA duplex in the presence of 10 mM Mg²⁺ or 0.5 mM Mn²⁺, wherein said RNase H is not recognized by antibody to human RNase H1 peptide fragments corresponding to amino acids 49-65 of the N-terminal region or amino acids 231-249 of the C-terminal region of SEQ ID NO: 1, or by antibody to full length human RNase H2.

2. The RNase H of claim 1 which is recognized by antibody to Fen1.

3. A method of cleaving the RNA strand of an RNA/DNA duplex comprising incubating said RNA/DNA duplex with a human RNase H of claim 1.

4. A method of cleaving the RNA strand of an RNA/DNA duplex comprising incubating said RNA/DNA duplex with a human RNase H of claim 2.

5. A method of cleaving the RNA strand of an RNA/DNA duplex comprising incubating said RNA/DNA duplex with immunoprecipitated Fen1.

6. The method of claim 5 wherein said immunoprecipitated Fen1 is human Fen1.

7. A method of cleaving the RNA strand of an RNA/DNA duplex comprising incubating said RNA/DNA duplex with cloned and expressed Fen1.

8. The method of claim 7 wherein said cloned and expressed Fen1 is human Fen1.

9. A method of enhancing inhibition of expression of a selected RNA by an antisense oligonucleotide targeted to said RNA, said method comprising:
(a) providing an antisense oligonucleotide targeted to an RNA whose expression is to be inhibited;
(b) allowing said oligonucleotide and said RNA to hybridize to form an oligonucleotide-RNA duplex;
(c) contacting said oligonucleotide-RNA duplex with a Fen1 polypeptide, under conditions in which cleavage of the RNA strand of the oligonucleotide-RNA duplex occurs, whereby inhibition of expression of the selected RNA is enhanced.

10. The method of claim 9 wherein the Fen1 is human Fen1.

11. The method of claim 9 wherein the Fen1 is immunoprecipitated Fen1.

12. The method of claim 9 wherein the Fen1 is cloned and expressed Fen1.

13. The method of claim 9 wherein the antisense oligonucleotide is a chimeric oligonucleotide.

14. A non-human mammalian cell comprising a nucleic acid encoding a human RNase H polypeptide.

15. The mammalian cell of claim 14 which is a mouse cell.

16. A mammalian cell which overexpresses human RNase H.

17. The mammalian cell of claim 16 which is a human cell.

18. The mammalian cell of claim 16 which is a mouse cell.

19. A non-human mammal comprising a nucleic acid encoding a human RNase H polypeptide.

20. The mammal of claim 19 which is a mouse.

21. A method of overexpressing a mammalian RNase H in a mammal, comprising inserting into said mammal a vector encoding a mammalian RNase H under conditions in which said mammalian RNase H is expressed in the mammal, wherein said mammalian RNase H is expressed at levels above endogenous levels for said RNase H in said mammal.

22. The method of claim 21 wherein said mammalian RNase H is a mammalian RNase H1.

23. The method of claim 21 wherein said mammalian RNase H is a mammalian RNase H2.

24. The method of claim 21 wherein said mammalian RNase H is a human RNase H.

25. The method of claim 21 wherein said mammalian RNase H is a wild type RNase H.

26. The method of claim 21 wherein said mammalian RNase H is a mutant RNase H.

27. The method of claim 26 wherein the mutant RNase H retains RNase H activity.

28. The method of claim 26 wherein the mutant RNase H is inactive.

29. The method of claim 28 wherein the inactive mutant is a dominant negative mutant.

30. The method of claim 21 wherein the vector is an adenovirus vector.

31. An antisense compound 8 to 80 nucleobases in length targeted to a nucleic acid molecule encoding human RNase H1, wherein said compound specifically hybridizes with said nucleic acid molecule encoding human RNase H1 and inhibits the expression of human RNase H1.

32. The compound of claim 31 which is 13 to 50 nucleobases in length.

33. The compound of claim 31 which is 15 to 30 nucleobases in length.

34. The compound of claim 31 comprising an oligonucleotide.

35. The compound of claim 34 comprising a DNA oligonucleotide.

36. The compound of claim 34 comprising an RNA oligonucleotide.

37. The compound of claim 31 comprising a chimeric oligonucleotide.

38. The compound of claim 31 which is a single-stranded compound.

39. The compound of claim 31 which is a fully or partially double-stranded compound.

40. The compound of claim 31 having at least one modified internucleoside linkage, sugar moiety, or nucleobase.

41. The compound of claim 40 having at least one 2′-O-methoxyethyl sugar moiety.

42. The compound of claim 40 having at least one phosphorothioate internucleoside linkage.

43. The compound of claim 40 having at least one 5-methylcytosine.

44. The compound of claim 31 which inhibits expression of human RNase H1 by at least 10%.

45. The compound of claim 31 which inhibits expression of human RNase H1 by at least 30%.

46. A method of inhibiting the expression of human RNase H1 in a cell or tissue comprising contacting said cell or tissue with the compound of claim 31 so that expression of human RNase H1 is inhibited.

47. A kit or assay device comprising the compound of claim 31.

48. The compound of claim 31, wherein said compound comprises SEQ ID NO: 78, 79, 80, 81, 82, 83, 84 or 85.

49. The compound of claim 31, wherein said compound comprises an antisense nucleic acid molecule that is specifically hybridizable with a 5′-untranslated region (5′UTR) of the nucleic acid molecule encoding human RNase H1.

50. The compound of claim 31, wherein said compound comprises an antisense nucleic acid molecule that is specifically hybridizable with a start region of the nucleic acid molecule encoding human RNase H1.

51. The compound of claim 31, wherein said compound comprises an antisense nucleic acid molecule that is specifically hybridizable with a coding region of the nucleic acid molecule encoding human RNase H1.

52. The compound of claim 31, wherein said compound comprises an antisense nucleic acid molecule that is specifically hybridizable with a 3′-untranslated region of the nucleic acid molecule encoding human RNase H1.

53. A double-stranded RNA compound comprising a sense strand having SEQ ID NO: 94, 95, 97, 98, 99, 100 or 105 and an antisense strand which is fully complementary to at least a 19-nucleobase region of the sense strand.

54. A method of isolating and purifying a cloned and expressed mammalian RNase H2 so that said RNase H2 retains its cleavage activity for the RNA strand of a RNA/DNA duplex substrate, comprising the steps of:
a) transfecting a cell with a vector encoding a mammalian RNase H2;
b) Overexpressing said mammalian RNase H2 in said cell;
c) Providing an antibody specific for said mammalian RNase H2;
d) Immunoprecipitating said RNase H2 from said cells using said antibody specific for said RNase H2 under conditions in which said mammalian RNase H2 retains cleavage activity for the RNA strand of a RNA/DNA duplex substrate.

55. The method of claim 54 wherein said transfected cell is a mammalian cell.

56. The method of claim 54 wherein said mammalian RNase H2 is a human RNase H2.

57. A substantially isolated and purified cloned and expressed mammalian RNase H2 which retains cleavage activity for the RNA strand of a RNA/DNA duplex substrate.

58. The mammalian RNase H2 of claim 57 which is a human RNase H2.


SEQUENCE LISTING (105 sequences)

SEQ ID NO: 1 - protein, 286 aa, Homo sapiens
  MISC_FEATURE (95)..(96): Xaa can be any naturally occurring amino acid
  MISC_FEATURE (112)..(112): Xaa can be any naturally occurring amino acid
  MISC_FEATURE (155)..(155): Xaa can be any naturally occurring amino acid
MSWLLFLAHRVALAALPCRRGSRGFGMFYAVRRGRKTGVFLTWNECRAQVDRFPAARFKKFATEDEAWAFVRKSASPEVSEGHENQHGQESEAKXXKRLREPLDGDGHESAXPYAKHMKPSVEPAPPVSRDTFSYMGDFVVVYTDGCCSSNGRRXPRAGIGVYWGPGHPLNVGIRLPGRQTNQRAEIHAACKAIEQAKTQNINKLVLYTDSMFTINGITNWVQGWKKNGWKTSAGKEVINKEDFVALERLTQGMDIQWMHVPGHSGFIGNEEADRLAREGAKQSED

SEQ ID NO: 2 - protein, 293 aa, Gallus gallus
MLRWLVALLSHSCFVSKGGGMFYAVRKGRQTGVYRTWAECQQQVNRFPSASFKKFATEKEAWAFVGAGPPDGQQSAPAETHGASAVAQENASHREEPETDVLCCNACKRRYEQSTNEEHTVRRAKHDEEQSTPVVSEAKFSYMGEFAVVYTDGCCSGNGRNRARAGIGVYWGPGHPLNISERLPGRQTNQRAEIHAACKAIEQAKSQNIKKLIIYTDSKFTINGITSWVENWKTNGWRTSSGGSVINKEDFQKLDSLSKGIEIQWMHIPGHAGFQGNEEADRLAREGASKQKL

SEQ ID NO: 3 - protein, 348 aa, yeast
MARQGNFYAVRKGRETGIYNTWNECKNQVDGYGGAIYKKFNSYEQAKSFLGQPNTTSNYGSSTHAGGQVSKPHTTQKRVHRRNRPLHYSSLTSSSACSSLSSANTNTFYSVKSNVPNIESKIFNNWKDCQAYVKHKRGITFKKFEDQLAAENFISGMSAHDYKLMNISKESFESKYKLSSNTMYNKSMNVYCDGSSFGNGTSSSRAGYGAYFEGAPEENISEPLLSGAQTNNRAEIEAVSEALKKIWEKLTNEKEKVNYQIKTDSEYVTKLLNDRYMTYDNKKLEGLPNSDLIVPLVQRFVKVKKYYELNKECFKNNGKFQIEWVKGHDGDPGNEMADFLAKKGASRR

SEQ ID NO: 4 - protein, 155 aa, E. coli
MLKQVEIFTDGSCLGNPGPGGYGAILRYRGREKTFSAGYTRTTNNRMELMAAIVALEALKEHCEVILSTDSQYVRQGITQWIHNWKKRGWKTADKKPVKNVDLWQRLDAALGQHQIKWEWVKGHAGHPENERCDELARAAAMNPTLEDTGYQVEV

SEQ ID NO: 5 - protein, 216 aa, Mus
GICGLGMFYAVRRGRRPGVFLSWSECKAQVDRFPAARFKKFATEDEAWAFVRSSSSPDGSKGQESAHEQKSQAKTSKRPREPLVVVYTDGCCSSNGRKRARAGIGVYWGPGHPLNVRIRLPGRQTNQRAEIHAACKAVMQAKAQNISKLVLYTDSMFTINGITNWVQGWKKNGWRTSTGKDVINKEDFMELDELTQGMDIQWMHIPGHSGFVGNEE

SEQ ID NO: 6 - protein, 299 aa, Homo sapiens [sequence omitted]

SEQ ID NO: 7 - protein, 128 aa, Mus sp.
MDLSELERDNTGRCRLSSPVPAVCLKEPCVLGVDEAGRGPVLGPMVYAICYCPLSRLADLEALKVADSKTLTENERERLFAKMEEDGDFVGWALDVLSPNLISTSMLGRVKYNLNSLSHDTAAGLIQY

SEQ ID NO: 8 - protein, 307 aa, Caenorhabditis elegans
SKTVKYFIERMSLKCETERSKTWNNFGNGIPCVLGIDEAGRGPVLGPMVYAAAISPLDQNVELKNLGVDDSKALNEAKREEIFNKMNEDEDIQQIIAYALRCLSPELISCSMLKRQKYSLNEVSHEAAITLIRDALACNVNVVEIKVDTVGPKATYQAKLEKLFPGISICVTEKADSLFPIVSAASIAAKVTRDSRLRNWQFREKNIKVPDAGYGSGYPGDPNTKKFLQLSVEPVFGFCSLVRSSWKTASTIVEKRCVPGSWEDDEEEGKSQSKRMTSWMVPKNETEVVPKRNMEINLTKIVSTLFL

SEQ ID NO: 9 - protein, 307 aa, Yeast
MVPPTVEASLESPYTKSYFSPVPSALLEQNDSPIIMGIDEAGRGPVLGPMVYAVAYSTQKYQDETIIPNYEFDDSKKLTDPIRRMLFSKIYQDNEELTQIGYATTCITPLDISRGMSKFPPTRNYNLNEQAHDVTMALIDGVIKQNVKLSHVYVDTVGPPASYQKKLEQRFPGVKFTVAKKADSLYCMVSVASVVAKVTRDILVESLKRDPDEILGSGYPSDPKTVAWLKRNQTSLMGWPANMVRFSWQTCQTLLDDASKNSIPIKWEEQYMDSRKNAAQKTKQLQLQMVAKPVRRKRLRTLDNWYR

SEQ ID NO: 10 - protein, 198 aa, E.coli
MIEFVYPHTQLVAGVDEVGRGPLVGAVVTAAVILDPARPIAGLNDSKKLSEKRRLALYEEIKEKALSWSLGRAEPHEIDELNILHATMLAMQRAVAGLHIAPEYVLIDGNRCPKLPMPAMAVVKGDSRVPEISAASILAKVTRDAEMAALDIVFPQYGFAQHKGYPTAFHLEKLAEHGATEHHRRSFGPVKRALGLAS

SEQ ID NO: 11 - DNA, 1131 bp, Homo sapiens
cgcgcctgcagtattagttcttgcagctggtggtggcggctgaggcggcatggatctcagcgagctggagagagacaatacaggccgctgtcgcctgagttcgcctgtgcccgcggtgtgccgcaaggagccttgcgtcctgggcgtcgatgaggcgggcaggggccccgtgctgggccccatggtctacgccatctgttattgtcccctgcctcgcctggcagatctggaggcgctgaaagtggcagactcaaagaccctattggagagcgagcgggaaaggctgtttgcgaaaatggaggacacggactttgtcggctgggcgctggatgtgctgtctccaaacctcatctctaccagcatgcttgggtgggtcaaatacaacctgaactccctgtcacatgatacagccactgggcttatacagtatgcattggaccagggcgtgaacgtcacccaggtattcgtggacaccgtagggatgccagagacataccaggcgcggctgcagcaaagttttcccgggattgaggtgacggtcaaggccaaagcagatgccctctacccggtggttagtgctgccagcatctgtgccaaggtggcccgggaccaggccgtgaagaaatggcaattcgtggagaaactgcaggacttggatactgattatggctcaggctaccccaatgatcccaagacaaaagcgtggttgaaggagcacgtggagcctgtgttcggcttcccccagtttgtccggttcagctggcgcacggcccagaccatcctggagaaagaggcggaagatgttatatgggaggactcagcatccgagaatcaggagggactcaggaagatcacatcctacttcctcaatgaagggtcccaagcccgtccccgttcttcccaccgatatttcctggaacgcggcctggagtcagcaaccagcctctagcagctgcctctacgcgctctacctgcttccccaacccagacattaaaattgtttaaggagaaccacacgtaggggatgtacttttgggacagaagcaaggtgggagtgtgctctgcagccgggtccagctacttccttttggaaccttaaatagaatgggtgttggttgataaaaaaaaaaa

SEQ ID NO: 12 - protein, 286 aa, Homo sapiens [sequence omitted]

SEQ ID NO: 13 - protein, 286 aa, Homo sapiens [sequence omitted]

SEQ ID NO: 14 - protein, 285 aa, Mus sp.
  ref <301>: AUTHORS: Cerritelli and Crouch
  ref <302>: TITLE: Cloning, Expression and Mapping of Ribonucleases H of Human and Mouse Related to Bacterial RNase HI
  ref <303>: JOURNAL: Genomics
  ref <304>: VOLUME: 53
  ref <305>: ISSUE: 3
  ref <306>: PAGES: 300-307
  ref <307>: DATE: 1998-11
MRWLLPLSRTVTLAVVRLRRGICGLGMFYAVRRGRRTGVFLSWSECKAQVDRFPAARFKKFATEDEAWAFVRSSSSPDGSKGQESAHEQKSQAKTSKRPREPLGEGEELPEPGPKHTRQDTEPAAVVSKDTFSYMGESVIVYTDGCCSSNGRKRARAGIGVYWGPGHPLNVGIRLPGRQTNQRAEIHAACKAIMQAKAQNISKLVLYTDSMFTINGITNWVQGWKKNGWRTSTGKDVINKEDFMELDELTQGMDIQWMHIPGHSGFVGNEEADRLAREGAKQSED

SEQ ID NO: 15 - protein, 298 aa, Homo sapiens
  ref <301>: AUTHORS: Frank, Braunshofer-Reiter, Wintersberger, Grimm and Busen
  ref <302>: TITLE: Cloning of the cDNA encoding the large subunit of human RNAse HI, a homologue of the prokaryotic RNAse HII
  ref <303>: JOURNAL: Proc. Natl. Acad. Sci. USA
  ref <304>: VOLUME: 95
  ref <305>: ISSUE: 22
  ref <306>: PAGES: 12872-12877
  ref <307>: DATE: 1998-10-27
MDLSELERDNTGRCRLSSPVPAVCRKEPCVLGVDEAGRGPVLGPMVYAICYCPLPRLADLEALKVADSKTLLESERERLFAKMEDTDFVGWALDVLSPNLISTSMLGRVKYNLNSLSHDTATGLIQYALDQGVNVTQVFVDTVGMPETYQAQLQQSFPGIEVTVKAKADALYPVVSAASICAKVARDQAVKKWQFVEKLQDLDTDYGSGYPNDPKTKAWLKEHVEPVFGFPQFVRFSWRTAQTILEKEAEDVIWEDSASENQEGLRKITSYFLNEGSQARPRSSHRYFLERGLESATS

SEQ ID NO: 16 - DNA, 26 bp, Artificial sequence
  Primer
acgctggccgggagtcgaaatgcttc

SEQ ID NO: 17 - DNA, 28 bp, Artificial sequence
  Primer
ctgttcctggcccacagagtcgccttgg

SEQ ID NO: 18 - DNA, 29 bp, Artificial sequence
  Primer
ggtctttctgacctggaatgagtgcagag

SEQ ID NO: 19 - DNA, 29 bp, Artificial sequence
  Primer
cttgcctggtttcgccctccgattcttgt

SEQ ID NO: 20 - DNA, 29 bp, Artificial sequence
  Primer
ttgattttcatgcccttctgaaacttccg

SEQ ID NO: 21 - DNA, 34 bp, Artificial sequence
  Primer
cctcatcctctatggcaaacttcttaaatctggc

SEQ ID NO: 22 - RNA, 17 nt, Artificial sequence
  RNA substrate
gggcgccgucggugugg

SEQ ID NO: 23 - DNA, 20 bp, Artificial sequence
  Primer
agcaggcgccgcttcgaggc

SEQ ID NO: 24 - DNA, 26 bp, Artificial sequence
  Primer
cccgctcctgcagtattagttcttgc

SEQ ID NO: 25 - DNA, 25 bp, Artificial sequence
  Primer
ttgcagctggtggtggcggctgagg

SEQ ID NO: 26 - DNA, 26 bp, Artificial sequence
  Primer
tccaatagggtctttgagtctgccac

SEQ ID NO: 27 - DNA, 25 bp, Artificial sequence
  Primer
cactttcagcgcctccagatctgcc

SEQ ID NO: 28 - DNA, 26 bp, Artificial sequence
  Primer
gcgaggcaggggacaataacagatgg

SEQ ID NO: 29 - DNA, 20 bp, Artificial sequence
  Antisense compound
cgcctcagccgccaccacca

SEQ ID NO: 30 - DNA, 20 bp, Artificial sequence
  Antisense compound
cacaggcgaactcaggcgac

SEQ ID NO: 31 - DNA, 20 bp, Artificial sequence
  Antisense compound
ggacaataacagatggcgta

SEQ ID NO: 32 - DNA, 20 bp, Artificial sequence
  Antisense compound
cccgctcgctctccaatagg

SEQ ID NO: 33 - DNA, 20 bp, Artificial sequence
  Antisense compound
cccagccgacaaagtccgtg

SEQ ID NO: 34 - DNA, 20 bp, Artificial sequence
  Antisense compound
cggtgtccacgaatacctgg

SEQ ID NO: 35 - DNA, 20 bp, Artificial sequence
  Antisense compound
cgcgcctggtatgtctctgg

SEQ ID NO: 36 - DNA, 20 bp, Artificial sequence
  Antisense compound
ggtagagggcatctgctttg

SEQ ID NO: 37 - DNA, 20 bp, Artificial sequence
  Antisense compound
ccaccttggcacagatgctg

SEQ ID NO: 38 - DNA, 20 bp, Artificial sequence
  Antisense compound
cagtttctccacgaattgcc

SEQ ID NO: 39 - DNA, 20 bp, Artificial sequence
  Antisense compound
ttttgtcttgggatcattgg

SEQ ID NO: 40 - DNA, 20 bp, Artificial sequence
  Antisense compound
agctgaaccggacaaactgg

SEQ ID NO: 41 - DNA, 20 bp, Artificial sequence
  Antisense compound
cctctttctccaggatggtc

SEQ ID NO: 42 - DNA, 20 bp, Artificial sequence
  Antisense compound
actccaggccgcgttccagg

SEQ ID NO: 43 - DNA, 20 bp, Artificial sequence
  Antisense compound
cctacgtgtggttctcctta

SEQ ID NO: 44 - DNA, 20 bp, Artificial sequence
  Antisense compound
gcacactcccaccttgcttc

SEQ ID NO: 45 - DNA, 20 bp, Artificial sequence
  Antisense compound
caaaaggaagtagctggacc

SEQ ID NO: 46 - DNA, 20 bp, Artificial sequence
  Antisense compound
aaacaattttaatgtctggg

SEQ ID NO: 47 - DNA, 20 bp, Artificial sequence
  Antisense compound
aattttaatgtctgggttgg

SEQ ID NO: 48 - DNA, 20 bp, Artificial sequence
  Antisense compound
ccttaaacaattttaatgtc

SEQ ID NO: 49 - protein, 20 aa, Artificial sequence
  Peptide fragment
CRAQVDRFPAARFKKFATED

SEQ ID NO: 50 - protein, 20 aa, Artificial sequence
  Peptide fragment
CKTSAGKEVINKEDFVALER

SEQ ID NO: 51 - DNA, 17 bp, Artificial sequence
  Enzyme substrate
ccacaccgacggcgccc

SEQ ID NO: 52 - RNA, 20 nt, Artificial sequence
  Enzyme substrate
acugugcguggaaagcguag

SEQ ID NO: 53 - DNA, 20 bp, Artificial sequence
  Enzyme substrate
ctacgctttccacgcacagt

SEQ ID NO: 54 - DNA, 21 bp, Artificial sequence
  Primer
agcttggaagacgatcagcaa

SEQ ID NO: 55 - DNA, 28 bp, Artificial sequence
  Primer
aaactgctgaactattgtaggagagatg

SEQ ID NO: 56 - DNA, 25 bp, Artificial sequence
  Probe
agatgccgtgtttgatggctccagc

SEQ ID NO: 57 - DNA, 26 bp, Artificial sequence
  Primer
aatggctaagtgaagatgacaatcat

SEQ ID NO: 58 - DNA, 25 bp, Artificial sequence
  Primer
tgcacatatcattacaccagttcgt

SEQ ID NO: 59 - DNA, 30 bp, Artificial sequence
  Probe
ttgcagcaattcactgtaaagctggaaagg

SEQ ID NO: 60 - DNA, 19 bp, Artificial sequence
  Primer
cgctggcctcagacacaga

SEQ ID NO: 61 - DNA, 24 bp, Artificial sequence
  Primer
ctaacctatcatcgacagccttca

SEQ ID NO: 62 - DNA, 24 bp, Artificial sequence
  Probe
agcagtcttgatgcctcgacggga

SEQ ID NO: 63 - DNA, 22 bp, Artificial sequence
  Primer
ggtttcctgctgccagatttaa

SEQ ID NO: 64 - DNA, 21 bp, Artificial sequence
  Primer
ggcttgcagatttcctgacaa

SEQ ID NO: 65 - DNA, 24 bp, Artificial sequence
  Probe
tttgccacagaggatgaggcctgg

SEQ ID NO: 66 - DNA, 19 bp, Artificial sequence
  Primer
cccgttcttcccaccgata

SEQ ID NO: 67 - DNA, 20 bp, Artificial sequence
  Primer
gctgctagaggctggttgct

SEQ ID NO: 68 - DNA, 20 bp, Artificial sequence
  Probe
ttcctggaacgcggcctgga

SEQ ID NO: 69 - DNA, 24 bp, Artificial sequence
  Primer
caacgtctggtatgatccttcaga

SEQ ID NO: 70 - DNA, 24 bp, Artificial sequence
  Primer
gtgctccctctcatctaactgctt

SEQ ID NO: 71 - DNA, 23 bp, Artificial sequence
  Probe
aagccccaccaccaaagatcccg

SEQ ID NO: 72 - DNA, 20 bp, Artificial sequence
  Antisense compound
tcccgcctgtgacatgcatt

SEQ ID NO: 73 - DNA, 20 bp, Artificial sequence
  Antisense compound
gctcagtggacatggatgag

SEQ ID NO: 74 - DNA, 20 bp, Artificial sequence
  Antisense compound
ctgctagcctctggatttga

SEQ ID NO: 75 - DNA, 20 bp, Artificial sequence
  Antisense compound
tgttgtcacgtttacttctg

SEQ ID NO: 76 - DNA, 20 bp, Artificial sequence
  Antisense compound
tccagcactttcttttccgg

SEQ ID NO: 77 - DNA, 1168 bp, Homo sapiens
ggaagtgcggtgttaagcgccggcggctcgcgcccacgctgggccgggagtcgaaatgcttcccggtgccgggagtgagcgatgagctggcttctgttcctggcccacagagtcgccttggccgccttgccctgccgccgcggctctcgcgggttcgggatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagcccggaagtttcagaagggcatgaaaatcaacatggacaagaatcggaggcgaaagccagcaagcgactccgtgagccactggatggagatggacatgaaagcgcagagccgtatgcaaagcacatgaagccgagcgtggagccggcgcctccagttagcagagacacgttttcctacatgggagacttcgtcgtcgtctacactgatggctgctgctccagtaatgggcgtagaaggccgcgagcaggaatcggcgtttactgggggccaggccatcctttaaatgtaggcattagacttcctgggcggcagacaaaccaaagagcggaaattcatgcagcctgcaaagccattgaacaagcaaagactcaaaacatcaataaactggttctgtatacagacagtatgtttacgataaatggtataactaactgggttcaaggttggaagaaaaatgggtggaagacaagtgcagggaaagaggtgatcaacaaagaggactttgtggcactggagaggcttacccaggggatggacattcagtggatgcatgttcctggtcattcgggatttataggcaatgaagaagctgacagattagccagagaaggagctaaacaatcggaagactgagccatgtgactttagtccttgggagaacttgagccagcggctgtcttgctgcctgtacttactggtgtggaaaatagcctgcaggtaggaccattgcagtgatgggcagatgcgtctttcacacggaatcaggcacagtggccttctgtgacatgtgtttataaaaaatggttaagtatataataaattgaacatcttttaaaaaaaaaaaaaaaaaaaaaaaaaa

SEQ ID NO: 78 - DNA, 20 bp, Artificial sequence
  Antisense compound
gcgcttaacaccgcacttcc

SEQ ID NO: 79 - DNA, 20 bp, Artificial sequence
  Antisense compound
catcgctcactcccggcacc

SEQ ID NO: 80 - DNA, 20 bp, Artificial sequence
  Antisense compound
cctcatcctctgtggcaaac

SEQ ID NO: 81 - DNA, 20 bp, Artificial sequence
  Antisense compound
cctccgattcttgtccatgt

SEQ ID NO: 82 - DNA, 20 bp, Artificial sequence
  Antisense compound
cgcccaggaagtctaatgcc

SEQ ID NO: 83 - DNA, 20 bp, Artificial sequence
  Antisense compound
tcttccaaccttgaacccag

SEQ ID NO: 84 - DNA, 20 bp, Artificial sequence
  Antisense compound
tggctcaagttctcccaagg

SEQ ID NO: 85 - DNA, 20 bp, Artificial sequence
  Antisense compound
tgcaggctattttccacacc

SEQ ID NO: 86 - DNA, 20 bp, Artificial sequence
  Antisense compound
cagtttctccacgaattgcc

SEQ ID NO: 87 - DNA, 20 bp, Artificial sequence
  Antisense compound
ttttgtcttgggatcattgg

SEQ ID NO: 88 - DNA, 20 bp, Artificial sequence
  Antisense compound
agctgaaccggacaaactgg

SEQ ID NO: 89 - DNA, 20 bp, Artificial sequence
  Antisense compound
cctctttctccaggatggtc

SEQ ID NO: 90 - DNA, 20 bp, Artificial sequence
  Antisense compound
actccaggccgcgttccagg

SEQ ID NO: 91 - DNA, 20 bp, Artificial sequence
  Antisense compound
cctacgtgtggttctcctta

SEQ ID NO: 92 - DNA, 20 bp, Artificial sequence
  Antisense compound
gcacactcccaccttgcttc

SEQ ID NO: 93 - DNA, 20 bp, Artificial sequence
  Antisense compound
caaaaggaagtagctggacc

SEQ ID NO: 94 - RNA, 21 nt, Artificial sequence
  dsRNA-sense strand
aaguuugccacagaggaugag

SEQ ID NO: 95 - RNA, 21 nt, Artificial sequence
  dsRNA-sense strand
aagccgagcguggagccggcg

SEQ ID NO: 96 - RNA, 21 nt, Artificial sequence
  dsRNA- sense strand
ggcaauucguggagaaacugc

SEQ ID NO: 97 - RNA, 21 nt, Artificial sequence
  dsRNA-sense strand
ccaaugaucccaagacaaaag

SEQ ID NO: 98 - RNA, 21 nt, Artificial sequence
  dsRNA-  sense strand
ccaguuuguccgguucagcug

SEQ ID NO: 99 - RNA, 21 nt, Artificial sequence
  dsRNA- sense strand
gaccauccuggagaaagaggc

SEQ ID NO: 100 - RNA, 21 nt, Artificial sequence
  dsRNA- sense strand
ccuggaacgcggccuggaguc

SEQ ID NO: 101 - RNA, 21 nt, Artificial sequence
  dsRNA-  sense strand
uaaggagaaccacacguaggg

SEQ ID NO: 102 - RNA, 21 nt, Artificial sequence
  dsRNA-  sense strand
gaagcaaggugggagugugcu

SEQ ID NO: 103 - RNA, 21 nt, Artificial sequence
  dsRNA- sense strand
gguccagcuacuuccuuuugg

SEQ ID NO: 104 - DNA, 20 bp, Artificial sequence
  control oligonucleotide. N = mixture of A,C,G,T
  misc_feature (1)..(20): N=mixture of A,C,T,G
nnnnnnnnnnnnnnnnnnnn

SEQ ID NO: 105 - RNA, 21 nt, Artificial Sequence
  dsRNA- sense strand
aaccaaugaucccaagacaaa